(19) United States
(12) Patent Application Publication
(10) Pub. No.: US 2015/0119435 A1
(43) Pub. Date: Apr. 30, 2015

(54) COMPOUNDS USEFUL FOR PROMOTING PROTEIN DEGRADATION AND METHODS USING SAME

(71) Applicant: YALE UNIVERSITY, NEW HAVEN, CT (US)

(72) Inventors: Craig Crews, NEW HAVEN, CT (US); Jeff Gustafson, ROCKY HILL, CT (US); Anke Gundula Roth, LEVENHAGEN (DE); Hyun Seop Tae, NEW HAVEN, CT (US); Dennis Buckley, NEW HAVEN, CT (US); Taavi Neklesa, ORANGE, CT (US)

(21) Appl. No.: 14/400,141

(22) PCT Filed: May 10, 2013
(86) PCT No.: PCT/US2013/040551

§ 371 Date: Nov. 10, 2014

Related U.S. Application Data
(60) Provisional application No. 61/785,161, filed on Mar. 14, 2013.
(60) Provisional application No. 61/645,914, filed on May 11, 2012.

(51) Int. Cl. C07D 233/86; C07C 43/196; C07C 233/21

(52) U.S. Cl. CPC C07D 233/86; C07C 233/21; C07C 43/196
USPC 514/391; 548/319.5; 564/188; 568/612

(57) ABSTRACT

The present invention includes compounds that act as degraders of a target protein, wherein degradation is independent of the class of the target protein or its localization. In certain embodiments, the invention comprises a compound comprising a protein degradation moiety covalently bound to a linker, wherein the ClogP of the compound is equal to or higher than 1.5. In other embodiments, the target protein contemplated within the invention comprises a posttranslational modified protein or intracellular protein. In yet other embodiments, compounds of the present invention are used to treat disease states wherein protein degradation is a viable therapeutic approach, such as cancer or any sort of oxidative stress disease state.

### CROSS-REFERENCE TO RELATED APPLICATIONS

The present application claims priority under 35 U.S.C. §119(e) to U.S. Provisional Application No. 61/645,914, filed May 11, 2012, and No. 61/785,161, filed Mar. 14, 2013, all of which applications are hereby incorporated by reference in their entireties herein.

### BACKGROUND OF THE INVENTION

There is great interest in removing and/or regulating endogenous proteins within a living organism. To date, most of these approaches have centered around RNAi and proteolysis targeting chimeric molecules (PROTACs), each of which has significant drawbacks that limit its therapeutic potential. For example, a strategy for post-translational protein degradation of fusion proteins containing Halo Tag-2, an engineered dehalogenase, was reported (Neklesa et al., 2011, Nat. Chem. Biol. 7(8):538-43). In this approach, a hydrophobic group is attached to the surface of HaloTag-2 via a haloalkane, which covalently binds to the active site of the mutant enzyme. The resulting modified protein mimics a partially denatured folding state, leading to the degradation of the fusion protein through the ubiquitin proteasome system. This method has limited therapeutic value due to its reliance on Halo Tag fusion proteins. Recently Hedstrom and coworkers (International Application No. WO 2012/003281) have extended this approach to include endogenous proteins and non-covalent linkages to the protein of interest, but this approach requires high concentrations of the bifunctional degron.

Some enzymes may be inhibited using natural products or small molecules, which bind the active sites of the enzyme, inhibiting its enzymatic activity. However, more than 80% of the entire proteome lack an active site. Thus, currently a significant number of key signaling proteins cannot be regulated using small molecules.

There have been reports of a novel chemical genetic strategy for protein degradation that is independent of protein class within the proteome and is based on small molecule modulators of protein function. These small molecule “perturbagens” can recruit the cellular protein quality control system to degrade the proteins of interest. Further, this chemical genetic approach can identify not only new enzymatic signaling components, but also non-enzymatic components (Schneekloth et al., 2004, JACS 126(12):3748-54).

The cellular control machinery, including the proteasome, removes partially denatured proteins, which are susceptible to aggregation. For example, this machinery is involved in the cellular response to heat shock. Elevated temperatures lead to a partial denaturation of the proteins, exposing hydrophobic residues usually sequestered in the core of the protein. Exposed hydrophobic residues change the character of the protein surface, inducing an intracellular response. Surveillance proteins such as heat shock protein-family (HSPs) chaperones can bind to partially denatured proteins, targeting them for proteasomal degradation. On the other hand, in collaboration with chaperonins, HSP's can refold proteins to their native state, preventing their proteasomal degradation.

Several diseased cells, especially tumor cells, are characterized by high levels of oxidized stress due to oncogenic stimulation, increased metabolic activity and mitochondrial malfunction. Reactive oxygen species (ROS) promote the oxidation of redox active cysteine residues in proteins. For example, ROS oxidizes exposed thiol groups to sulfenic acid.

Sulfenic acid has emerged as a biologically relevant post-translational modification in a number of disease states or conditions where oxidative stress is implicated, including cancer. The term “sulfenome” describes a collection of proteins comprising sulfenic acid residues (most likely derived from oxidation of cysteinyl residues) found in cells undergoing oxidative stress. The “sulfenome” is likely not only much larger in cancer cells in comparison with healthy cells, but also a significant portion of the entire proteome in cancer cells. An analysis of the sulfenome of HeLa cells identified more than 180 potential sulfenic acid-modified proteins with roles in signal transduction, protein synthesis, redox homeostasis, DNA repair and ER quality control. The huge advantage of targeting the “sulfenome” is that it does not correspond to a specific protein, but rather comprises multiple cancer-related targets. In fact, common but unique patterns of sulfenic acid modifications in different subtypes of human breast cancer cell lines have been identified.

There is a need in the art for identifying compounds that promote controlled degradation of specific target proteins. These compounds should function in a manner that is independent from the protein class or protein localization. These compounds are useful for treating disease states wherein protein degradation represents a viable therapy approach (such as oxidative stress disease states or cancer). The present invention addresses and meets these needs.

### BRIEF SUMMARY OF THE INVENTION

The invention includes a composition comprising a compound of formula (I), or a pharmaceutically acceptable salt, solvate or polymorph thereof: L-DM (I), wherein: DM is a protein degradation moiety; L is a linker, wherein L is covalently bound to DM; L-DM has a ClogP value equal to or higher than about 1.5; and, L comprises a functional group that is capable of forming a covalent bond to a protein binding moiety (PBM).

In one embodiment, L-DM has a ClogP value equal to or higher than about 2.0. In another embodiment, L-DM has a ClogP value equal to or higher than about 3.0. In yet another embodiment, DM is selected from the group consisting of:

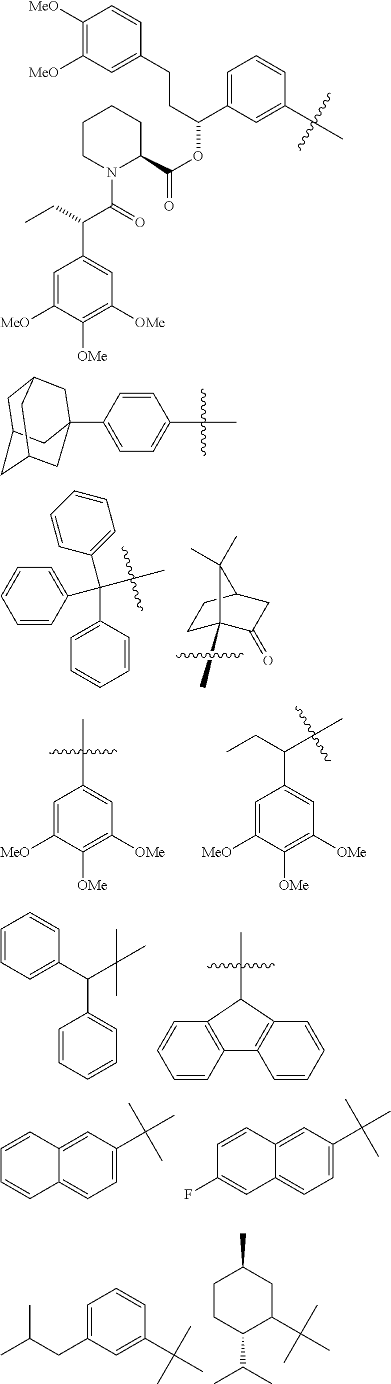

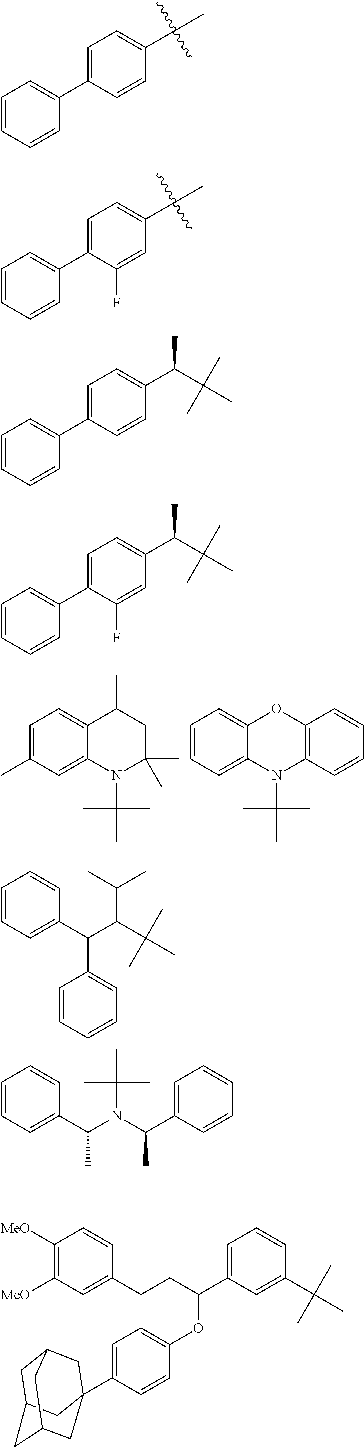

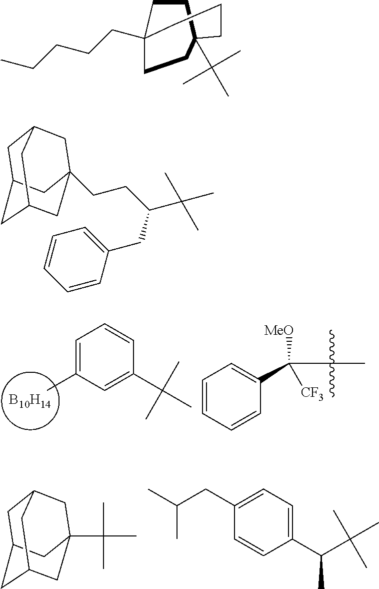

In yet another embodiment, L ranges in length from 2 to 60 atoms. In yet another embodiment, L ranges in length from 2 to 8 atoms. L comprises from 1 to 15 ethylene oxide groups.

In one embodiment, L comprises a group of formula (II): —[Z—X—Y^{R}]— (II), wherein Z links PBM to X; X links Z to group Y^{R}; and Y^{R} links to DM, further wherein: Z and Y^{R} are independently a bond, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, —(CH₂)ᵢ—C≡C—, or —Y—C(O)—Y—; X is -(D-CON-D)ᵢ-, wherein each occurrence of D is independently a bond, —(CH₂)ᵢ—Y—C(═O)—Y—(CH₂)ᵢ—, —(CH₂)ᵢ— or —[(CH₂)ᵢ—X¹]ᵢ—; X¹ is O, S or N—R⁴; CON is a bond, —C(O)NH—, —NH(CO)—, —X²—, —X³—C(O)—X³—,

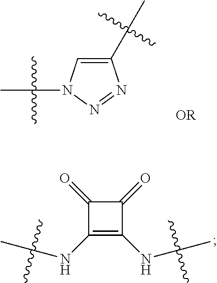 [SMILES: *c1cn(*)nn1 ; *Nc1c(N*)c(=O)c1=O]

X² is —O—, —S—, —N(R⁴)—, —S(O)—, —S(O)₂—, —S(O)₂O—, —OS(O)₂, or OS(O)₂O; X³ is O, S, or NR⁴; R⁴ is H or C₁-C₃ alkyl; each occurrence of ‘i’ is independently an integer ranging from 0 to 100; each occurrence of Y is independently a bond, O, S, —N(R^{N})—, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, or —(CH₂)ᵢ—C≡C—; each occurrence of R^{N} is independently H, C₁-C₃ alkyl or hydroxylated C₁-C₃ alkyl; and, XY is —C(O)NH—, —NHC(O), —OC(O)NH—, —NHC(O)O—, —C(O)O—, —OC(O)—, —C(O)S—, or —SC(O).

In one embodiment, each occurrence of ‘i’ is independently an integer ranging from 1 to 8. In another embodiment, CON is —C(O)NH—, —NH(CO)—, or

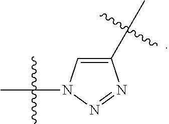 [SMILES: *c1cn(*)nn1]

In one embodiment, the compound of formula (I) is selected from the group consisting of:

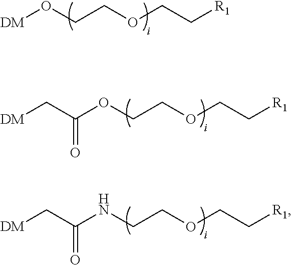

wherein R₁ is COOH, CHO, SH, OH or NH₂, and ‘i’ is an integer ranging from 1 to 100.

In one embodiment, the compound of formula (I) is selected from the group consisting of:

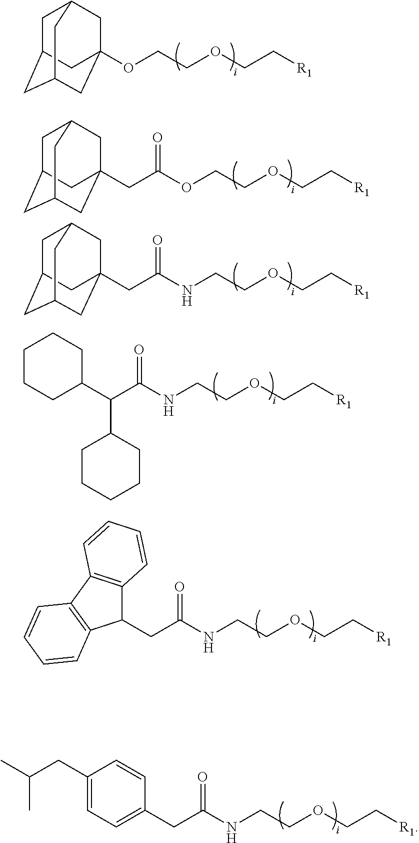

In one embodiment, L is further covalently bound to a protein binding moiety (PBM), whereby the compound of formula (I) is the compound of formula (Ia) or a pharmaceutically acceptable salt, solvate or polymorph thereof: PBM-L-DM (Ia). In another embodiment, the PBM binds to at least one target protein selected from the group consisting of an androgen receptor, sulfenic acid—comprising protein, a neurofibrillary tangle, and any combinations thereof.

In one embodiment, the PBM is selected from the group consisting of:

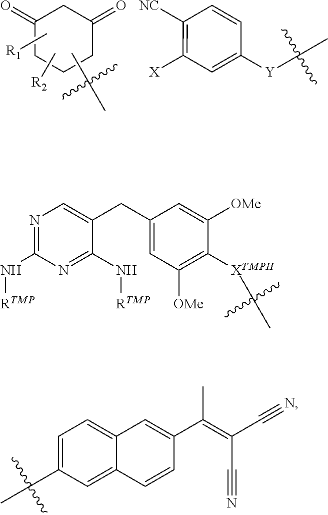

wherein: each occurrence of R¹ and R² is independently selected from the group consisting of H, substituted C₁-C₆ alkyl, substituted C₂-C₆ alkynyl, —C(O)(C₁-C₆ alkyl), —NO₂, —CN, —F, —Cl, —Br, —I, —CF₃, —C(O)CF₃ and —C≡C—Rₐ, wherein each alkyl or alkynyl group is optionally and independently substituted with 1-6 electron withdrawing groups; Rₐ is H or C₁-C₆ alkyl; X is NO₂, CN, F, Cl, Br, I, —C≡C—Rₐ, CF₃, or —C(O)CF₃; Y is a bond,

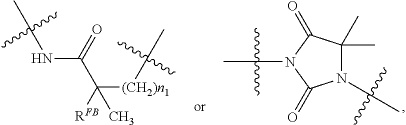

wherein R^{FB} is H or OH, and n₁ is 0, 1, 2, or 3; each occurrence of R^{TMP} is independently H, C₁-C₂₀ alkyl or C₁-C₂₀ acyl; X^{TMP} is O, S, S(O)₂, CH₂ or NR^{FB1}; each occurrence of R^{FB1} is independently H or a C₁-C₃ alkyl group substituted with 1-3 hydroxyl groups; and, each occurrence of n₂ is independently 0, 1, 2, or 3. In another embodiment, R¹ and R² are H.

In one embodiment, the composition further comprises a pharmaceutically acceptable carrier. In another embodiment, the composition further comprises a bioactive agent. In yet another embodiment, the bioactive agent comprises a HSP90 modulator or a HSP70 modulator. In yet another embodiment, the HSP90 modulator comprises geldanamycin, 17AAG/KOS953, 17-DMAG, CNF1010, tanespimycin, alvespimycin, KOS 1022, retaspimycin or 17-AAG hydroquinone, KOSN 1559, PU3, PUH58, PU24S, PU24FC1, BIIB021, CCT018159, G3219, G3130, VER49009/CCT0129397, VER50589, STA-9090, VER52296/NVP-AUY922, SNS2112, SNX5422, radicicol, cyclproparadicicol, KF 25706, KF 55823, novobiocin, chlorobiocin, coumermycin A1, coumermycin compound A4, DHN2, KU135, 4TCNA, 4TDHCNA, 4TTCQ, or CUDC-305. In yet another embodiment, the HSP70 modulator comprises 2-phenylethynesulfonamide (PES) or geranylgeranylacetone.

The invention also includes a method of degrading or inhibiting a target protein in a subject in need thereof. The method comprises administering to the subject an effective amount of a pharmaceutically acceptable composition comprising a compound of formula (Ia) or a pharmaceutically acceptable salt, solvate or polymorph thereof: PBM-L-DM (Ia), wherein DM is a protein degradation moiety; L is a linker, wherein L is covalently bound to DM; L-DM has a total ClogP value equal to or higher than about 1.5; L is covalently bound to a protein binding moiety (PBM); and PBM binds to the target protein; whereby the target protein is degraded or inhibited in the subject.

In one embodiment, the target protein comprises B7, B7-1, TINFR1m, TNFR2, NADPH oxidase, BclIBax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, histamine receptors, 5-lipoxygenase, tryptase serine protease, thymidylate synthase, purine nucleoside phosphorylase, GAPDH trypanosomal, glycogen phosphorylase, carbonic anhydrase, chemokine receptors, JAW STAT, RXR, HIV 1 protease, HIV 1 integrase, influenza neuramimidase, hepatitis B reverse transcriptase, sodium channel, protein P-glycoprotein (and MRP), tyrosine kinases, CD23, CD124, tyrosine kinase p56 lck, CD4, CD5, IL-2 receptor, IL-1 receptor, TNF-alphaR, ICAM1, Ca++ channels, VCAM, VLA-4 integrin, selectins, CD40/CD40L, newokinins and receptors, inosine monophosphate dehydrogenase, p38 MAP Kinase, Ras1Raf1MEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, tumor associated protein (TMP), Bcr-Abl tyrosine kinase, cytosolic phospholipaseA2, EGF receptor tyrosine kinase, ecdysone 20-monooxygenase, ion channel of the GABA gated chloride channel, acetylcholinesterase, voltage-sensitive sodium channel protein, calcium release channel, chloride channels, acetyl-CoA carboxylase, adenylosuccinate synthetase, protoporphyrinogen oxidase, enolpyruvylshikimate-phosphate synthase, or drug resistant and multiple drug resistance (MDR) proteins.

In one embodiment, the target protein comprises Bcr-Abl tyrosine kinase, dihydrofolate reductase, p38 kinase, checkpoint kinase 2, RAF kinase, VEGFR2, VEGFR3, ALK (anaplastic lymphoma kinase), Aurora kinase, Janus kinase 2 (JAK2), protein tyrosine phosphatase, SHP-2 domain of protein tyrosine phosphatase, mitogen activated protein kinase (BRAF^{V600E}/MEK), MDM2 ubiquitin ligase, human BET bromodomain-containing protein Brd2, Brd3, Brd4, and Skpl-Cullin-F box complex, HSP90, HSP70, VEGF, ubiquitin ligase, histone deacetylase protein (HDAC), lysine methyltransferase, aryl hydrocarbon receptor, estrogen receptor, FK506 binding protein (FKBP), thyroid hormone receptor (THR), HIV protease, HIV integrase, HCV protease, acyl-protein thioesterase 1 and 2 (APT1 and APT2), tyrosine kinase p56 lck, EGF receptor tyrosine kinase, tyrosine kinase Flk-IIKDR or tumor associated membrane protein (TMP).

In one embodiment, the subject is further administered a bioactive agent. In another embodiment, the bioactive agent comprises a HSP90 modulator or a HSP70 modulator. In yet another embodiment, the HSP90 modulator comprises geldanamycin, 17AAG/KOS953, 17-DMAG, CNF1010, tanespimycin, alvespimycin, KOS 1022, retaspimycin or 17-AAG hydroquinone, KOSN 1559, PU3, PUH58, PU24S, PU24FC1, BIIB021, CCT018159, G3219, G3130, VER49009/CCT0129397, VER50589, STA-9090, VER52296/NVP-AUY922, SNS2112, SNX5422, radicicol, KF 25706, KF 55823, cyclproparadicicol, novobiocin, chlorobiocin, coumermycin A1, coumermycin compound A4, DHN2, KU135, 4TCNA, 4TDHCNA, 4TTCQ, or CUDC-305. In yet another embodiment, the HSP70 modulator comprises 2-phenylethynesulfonamide (PES) or geranylgeranylacetone.

In one embodiment, the subject is a mammal. In another embodiment, the mammal is human.

The invention also includes a method of treating or preventing a disease or disorder in a subject in need thereof, wherein the disease or disorder is associated with a target protein in the subject. The method comprises administering to the subject a therapeutically effective amount of a pharmaceutical composition comprising a compound of formula (Ia) or a pharmaceutically acceptable salt, solvate or polymorph thereof: PBM-L-DM (Ia), wherein DM is a protein degradation moiety; L is a linker, wherein L is covalently bound to DM; L-DM has a total ClogP value equal to or higher than about 1.5; L is covalently bound to a protein binding moiety (PBM), and PBM binds to the target protein; whereby the disease or disorder is treated or prevented in the subject.

In one embodiment, the disease or disorder comprises asthma, autoimmune diseases, cancers, ciliopathies, cleft palate, diabetes, heart disease, hypertension, inflammatory bowel disease, mental retardation, mood disorder, obesity, refractive error, infertility, Angelman syndrome, Canavan disease, coeliac disease, Charcot-Marie-Tooth disease, cystic fibrosis, duchenne muscular dystrophy, haemochromatosis, haemophilia, Klinefelter's syndrome, neurofibromatosis, phenylketonuria, polycystic kidney disease, (PKD1) or 4 (PKD2) Prader-Willi syndrome, sickle-cell disease, Tay-Sachs disease, Turner syndrome, Alzheimer's disease, amyotrophic lateral sclerosis (Lou Gehrig's disease), anorexia nervosa, anxiety disorder, atherosclerosis, attention deficit hyperactivity disorder, autism, bipolar disorder, chronic fatigue syndrome, chronic obstructive pulmonary disease, Crohn's disease, coronary heart disease, dementia, depression, diabetes mellitus type 1, diabetes mellitus type 2, epilepsy, Guillain-Barré syndrome, irritable bowel syndrome, lupus, metabolic syndrome, multiple sclerosis, myocardial infarction, obesity, obsessive-compulsive disorder, panic disorder, Parkinson's disease, psoriasis, rheumatoid arthritis, sarcoidosis, schizophrenia, stroke, thromboangiitis obliterans, Tourette syndrome, vasculitis, aceruloplasminemia, achondrogenesis type II, achondroplasia, acrocephaly, Gaucher disease type 2, acute intermittent porphyria, Canavan disease, adenomatous Polyposis Coli, ALA dehydratase deficiency, adenylosuccinate lyase deficiency, adrenogenital syndrome, adrenoleukodystrophy, ALA-D porphyria, ALA dehydratase deficiency, alkaptonuria, Alexander disease, alkaptonuric ochronosis, alpha 1-antitrypsin deficiency, alpha-1 proteinase inhibitor, emphysema, amyotrophic lateral sclerosis, Alström syndrome, Alexander disease, Amelogenesis imperfecta, ALA dehydratase deficiency, Anderson-Fabry disease, androgen insensitivity syndrome, anemia, angiokeratoma corporis diffusum, angiomatosis retinae (von Hippel-Lindau disease), Apert syndrome, arachnodactyly (Marfan syndrome), Stickler syndrome, arthrochalasis multiplex congenital (Ehlers-Danlos syndrome#arthrochalasia type), ataxia telangiectasia, Rett syndrome, primary pulmonary hypertension, Sandhoff disease, neurofibromatosis type II, Beare-Stevenson cutis gyrata syndrome, mediterranean fever, familial, Benjamin syndrome, beta-thalassemia, bilateral acoustic neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndrome (Rubinstein-Taybi syndrome), bronze diabetes/bronzed cirrhosis (hemochromatosis), bulbospinal muscular atrophy (Kennedy's disease), Burger-Grutz syndrome (lipoprotein lipase deficiency), CGD chronic granulomatous disorder, campomelic dysplasia, biotinidase deficiency, cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, chondrodystrophy syndrome (achondroplasia), otospondylomegaepiphyseal dysplasia, Lesch-Nyhan syndrome, galactosemia, Ehlers-Danlos syndrome, thanatophoric dysplasia, Coffin-Lowry syndrome, Cockayne syndrome (familial adenomatous polyposis), congenital erythropoietic porphyria, congenital heart disease, methemoglobinemia/congenital methaemoglobinaemia, achondroplasia, X-linked sideroblastic anemia, connective tissue disease, conotruncal anomaly face syndrome, Cooley's Anemia (beta-thalassemia), copper storage disease (Wilson's disease), copper transport disease (Menkes disease), hereditary coproporphyria, Cowden syndrome, craniofacial dysarthrosis (Crouzon syndrome), Creutzfeldt-Jakob disease (prion disease), Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, degenerative nerve diseases including de Grouchy syndrome and Dejerine-Sottas syndrome, developmental disabilities, distal spinal muscular atrophy, type V, androgen insensitivity syndrome, diffuse globoid body sclerosis (Krabbe disease), Di George's syndrome, dihydrotestosterone receptor deficiency, androgen insensitivity syndrome, Down syndrome, dwarfism, erythropoietic protoporphyria, erythroid 5-aminolevulinate synthetase deficiency, erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), fibrocystic disease of the pancreas, fragile X syndrome, galactosemia, genetic brain disorders, giant cell hepatitis (neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), hyperandrogenism, hypochondroplasia, hypochromic anemia, immune system disorders, Insley-Astley syndrome, Jackson-Weiss syndrome, Joubert syndrome, Lesch-Nyhan syndrome, Jackson-Weiss syndrome, kidney diseases, including hyperoxaluria, Klinefelter's syndrome, Kniest dysplasia, lacunar dementia, Langer-Saldino achondrogenesis, ataxia telangiectasia, Lynch syndrome, lysyl-hydroxylase deficiency, Machado-Joseph disease, metabolic disorders, Marfan syndrome, movement disorders, Mowat-Wilson syndrome, Muenke syndrome, multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford progeria syndrome), progressive chorea, chronic hereditary (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), recurrent polyserositis, retinal disorders, retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, skin pigmentation disorders, Smith-Lemli-Opitz syndrome, South-African genetic porphyria (variegate porphyria), infantile-onset ascending hereditary spastic paralysis, speech and communication disorders, sphingolipidosis, spinocerebellar ataxia, Stickler syndrome, stroke, androgen insensitivity syndrome, tetrahydrobiopterin deficiency, beta-thalassemia, thyroid disease, tomaculous neuropathy (hereditary neuropathy with liability to pressure palsies), Treacher Collins syndrome, triplo X syndrome (triple X syndrome), trisomy 21 (Down syndrome), trisomy X, VHL syndrome (von Hippel-Lindau disease), vision impairment and blindness (Alström syndrome), Vrolik disease, Waardenburg syndrome, Warburg Sjo Fledelius Syndrome, Weissenbacher-Zweymüiller syndrome, Wolf-Hirschhorn syndrome, Wolff periodic disease, Weissenbacher-Zweymüiller syndrome or Xeroderma pigmentosum.

In one embodiment, the disease or disorder comprises cancer. In another embodiment, the cancer comprises squamous-cell carcinoma, basal cell carcinoma, adenocarcinoma, hepatocellular carcinomas, renal cell carcinomas, cancer of the bladder, bowel, breast, cervix, colon, esophagus, head, kidney, liver, lung, neck, ovary, pancreas, prostate, and stomach; leukemias; benign and malignant lymphomas; benign and malignant melanomas; myeloproliferative diseases; sarcomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor or teratocarcinomas.

In one embodiment, the subject is further administered an anticancer agent. In another embodiment, the anticancer agent comprises everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulat/or, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, (pyro-Glu-His-Trp-Ser-Tyr-D-Ser(Bu t)-Leu-Arg-Pro-Azgly-NH₂ acetate [C₅H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, or darbepoetin alfa.

In one embodiment, the subject is further administered a bioactive agent. In another embodiment, the bioactive agent comprises a HSP90 modulator or a HSP70 modulator. In yet another embodiment, the HSP90 modulator comprises geldanamycin, 17AAG/KOS953, 17-DMAG, CNF1010, tanespimycin, alvespimycin, KOS 1022, retaspimycin or 17-AAG hydroquinone, KOSN 1559, PU3, PUH58, PU24S, PU24FC1, BIIB021, CCT018159, G3219, G3130, VER49009/CCT0129397, VER50589, STA-9090, VER52296/NVP-AUY922, SNS2112, SNX5422, radicicol, KF 25706, KF 55823, cyclproparadicicol, novobiocin, chlorobiocin, coumermycin A1, coumermycin compound A4, DHN2, KU135, 4TCNA, 4TDHCNA, 4TTCQ, or CUDC-305. In yet another embodiment, the HSP70 modulator comprises 2-phenylethynesulfonamide (PES) or geranylgeranylacetone.

In one embodiment, the subject is a mammal. In another embodiment, the mammal is human.

The invention also includes a method of treating or preventing an oxidative stress disease state or condition in a subject in need thereof. The method comprises administering to the subject a therapeutically effective amount of a pharmaceutical composition comprising a compound of formula (Ia) or a pharmaceutically acceptable salt, solvate or polymorph thereof: PBM-L-DM (Ia), wherein DM is a protein degradation moiety; L is a linker, L being covalently bound to DM; L-DM has a ClogP value equal to or higher than about 1.5; L is covalently bound to a protein binding moiety (PBM), and PBM binds to the target protein; whereby the disease state or condition is treated or prevented in the subject.

In one embodiment, the oxidative stress disease state or condition comprises cancer, hyperproliferative cell growth conditions, Parkinson's disease, Alzheimer's disease, atherosclerosis, heart failure, including congestive heart failure, myocardial infarction, schizophrenia, bipolar disorder, fragile X syndrome, sick cell disease, chronic fatigue syndrome, aging (including aging by induction of mitohormesis, diabetes (especially type I) or vascular disease.

In one embodiment, the subject is further administered a bioactive agent. In another embodiment, the bioactive agent comprises a HSP90 modulator or a HSP70 modulator. In yet another embodiment, the HSP90 modulator comprises geldanamycin, 17AAG/KOS953, 17-DMAG, CNF1010, tanespimycin, alvespimycin, KOS 1022, retaspimycin or 17-AAG hydroquinone, KOSN 1559, PU3, PUH58, PU24S, PU24FC1, BIIB021, CCT018159, G3219, G3130, VER49009/CCT0129397, VER50589, STA-9090, VER52296/NVP-AUY922, SNS2112, SNX5422, radicicol, KF 25706, KF 55823, cyclproparadicicol, novobiocin, chlorobiocin, coumermycin A1, coumermycin compound A4, DHN2, KU135, 4TCNA, 4TDHCNA, 4TTCQ, or CUDC-305. In yet another embodiment, the HSP70 modulator comprises 2-phenylethynesulfonamide (PES) or geranylgeranylacetone.

In one embodiment, the subject is a mammal. In another embodiment, the mammal is human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1B, is a non-limiting illustration of a hydrophobic tag strategy for inducing proteasomal degradation, wherein small molecules are used to control the intracellular protein levels. FIG. 1A: Chaperonins recognize proteins that are partially denatured (due to, for example, heat shock or oxidative stress) and either assist in their refolding or target them for proteasome-mediated degradation. FIG. 1B: A heterobifunctional molecule of the invention, comprising a hydrophobic tag coupled to a protein recognition group, “tags” the target protein and induce its degradation.

FIG. 2 is a non-limiting illustration of the oxidation states of the amino acid cysteine within proteins. The initial oxidation product of cysteine is sulfenic acid, which is implicated in a wide range of biological functions.

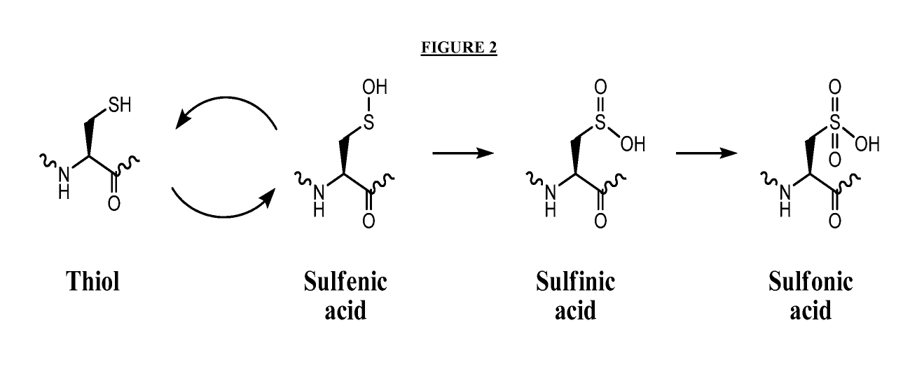

FIG. 3, comprising FIGS. 3A-3B, illustrates hydrophobic moieties (degrons) useful within the invention. In one embodiment, the hydrophobic group is covalently bound to a linker. In another embodiment, the linker is further covalently bound to a protein binding moiety.

FIG. 4 is a non-limiting illustration of a general chemical synthetic approach useful within the invention. In this non-limiting embodiment, an ethyleneglycol adamantyl-derived intermediate is converted to an activated N-hydroxysuccinimide derivative and condensed with a protein binding moiety that has a free amine group, thus forming a bifunctional compound.

FIG. 5 is a scheme illustrating the chemical synthesis of ethyleneglycol adamantyl-derived (linker-degron) intermediates. Conditions: a) i. Ms-Cl, Ag₂O, CH₂Cl₂; ii. NaN₃, DMF, 110° C.; iii. PPh₃, THF, 0° C.; b) NaH, 1-5, DMF; c) R=I: PPh₃, imidazole, I₂; R=NH₂: i) Tos-Cl, NEt₃, THF ii) NaN₃, DMF, iii) H₂, Pd/C; d) alcohol 1-5 or L1-L5, DCC, DMAP, DCM; e) L1-L5, DCC, DMAP, DCM.

FIG. 6 is a scheme illustrating the synthesis of covalent adamantyl-derived hydrophobic tags for oxidized cysteine residues within proteins.

FIG. 7 is a scheme illustrating the general synthesis of hydrophobic amide/amide dimedone derivatives.

FIG. 8 is a scheme illustrating the general synthesis of hydrophobic ester/ester dimedone derivatives.

FIG. 9, comprising FIGS. 9A-9B, is a scheme illustrating the general synthesis of amide/ester hydrophobic dimedone derivatives using a non-protected (FIG. 9A) strategy and a protected (FIG. 9B) strategy.

FIG. 10 is a scheme illustrating a synthesis of hydrophobic ether/ester and ether/amide dimedone derivatives.

FIG. 11, comprising FIGS. 11A-11B, illustrates compounds contemplated within the invention. FIG. 11A is a scheme illustrating the compounds used in a structure activity relationship investigation with dimedone as protein binding moiety, using varying protein degradation moieties or degrons. FIG. 11B is a scheme illustrating the general synthesis of related compounds lacking the protein binding group; such compounds may be used for determining impact of the degron on biological activity.

FIG. 12 is a scheme illustrating the synthesis of a control compound comprising a cyclohexanine group.

FIG. 13 is a scheme illustrating the general synthesis of a pulldown reagent (AGR 213), which comprises a dimedone group and an adamantyl group and is amenable for further derivatization.

FIG. 14 is a scheme illustrating the general synthesis of a pulldown reagent (AGR 248), which is amenable for further derivatization.

FIG. 15, comprising FIGS. 15A-15B, illustrates experimental results with selected SARD compounds. FIG. 15A illustrates representative western blots of SARD mediated AR degradation. FIG. 15B is a graph illustrating the percentage of remaining AR as a function of SARD compound concentration.

FIG. 16 is a scheme illustrating the synthesis of TMP-based (tumor associated membrane protein) compounds and representative TMP based degraders (TMP binding group-linker-degron).

FIG. 17 is a scheme illustrating compounds of the invention, including compound AGR054.

FIG. 18 is a graph illustrating in vitro activities of dimedone derivatives in HeLa cells.

FIG. 19 is a series of photographs illustrating the treatment of HeLa cells with AGR054 (100 μM) and AGR118 (500 μM) for 8 hours (top row) and the treatment of HeLa cells with AGR054 (250 μM) and AGR118 (250 μM) for 4 hours (bottom row).

FIG. 20 is a set of graphs illustrating the induction of apoptosis by dimedone-degron compounds. Shown is the cellular DNA content (PI staining). Top row left: HeLa cells with AGR054 (75 μM) and DMSO (control), respectively, for 24 hours. Top row right: Jurkat T cells with AGR054 (75 μM) and DMSO (control) for 24 hours. Bottom Row: Western Blot of HeLa cells, AGR054 concentration as indicated in figure for 8 hours.

FIG. 21 is a graph illustrating the finding that dimedone-degron compound AGR054 rapidly increases intracellular ROS levels. Shown is the level of DCF fluorescence for different concentrations of AGR054 (and controls) in HeLa cells after a 1 hour treatment.

FIG. 22 is a graph illustrating the biological activity of AGR054 and AGR181 in HeLa cells (measured after 24 hours of treatment). Control compound (AGR181, diamond dots) lacking the 1,3-diketone scaffold was inactive.

FIG. 23 illustrates pull down reagents of the invention for identifying proteins. The reagents may be used within the invention or may be used as intermediates in chemical synthesis of other compounds of the invention. As an example is shown a dimedone derivative, which comprises an adamantyl group attached through a linker to biotin.

FIG. 24 illustrates a number of representative SARDS analogs of the invention with varying protein degradation moieties (degrons).

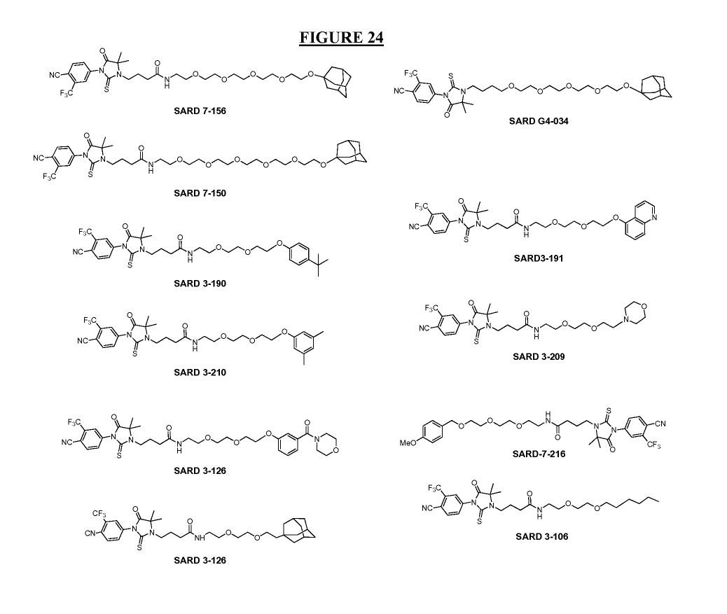

FIG. 25 illustrates non-limiting examples of Abl kinase degrader compounds based upon imatinib (Gleevac) moieties. The pharmacological fragment of imatinib is derivatized with distinct linkers and degrons using facile acylation chemistry or by simply condensing a linker onto an imatinib protein binding moiety, as indicated.

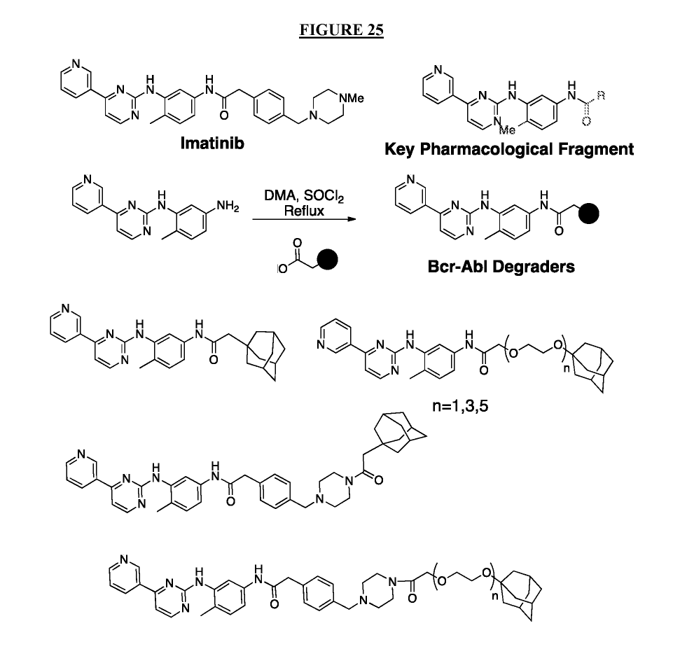

FIG. 26, comprising FIGS. 26A-26C, is a series of graphs illustrating in vitro activity of SARDS compounds. FIG. 26A: effect of SARDS on cell proliferation of LnCAPs. FIG. 26B: effect of SARDS on the proliferation of non-AR dependent cell lines. FIG. 26C: effect of SARDS on androgen independent prostate cancer cells.

FIG. 27 is the reproduction of a gel that illustrates that compounds (SARDs) of the present invention work additively with a HSP90 inhibitor Inhibition of HSP90, to which the androgen receptor (AR) is bound, results in greater AR instability upon co-administration of SARD degraders along with geldanamycin.

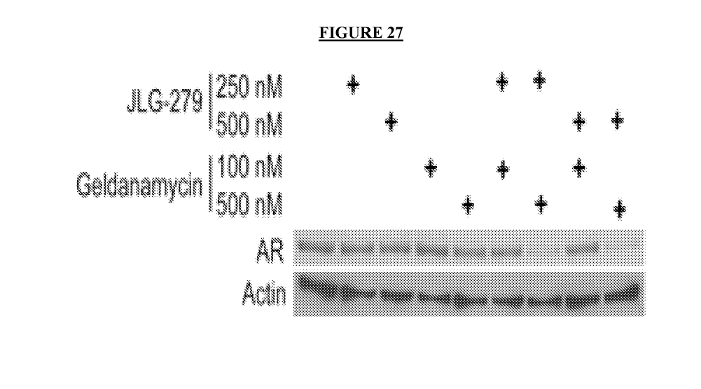

FIG. 28 illustrates a compound of the invention comprising a protein targeting moiety that binds to the dihydrofolate reductase (DHFR) enzyme (top) and its biological effect in suppressing expression of E. coli DHFR in mammalian cells.

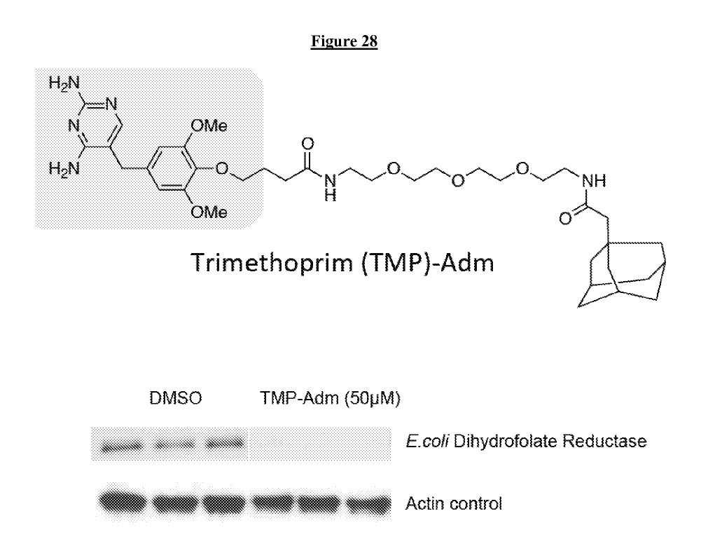

FIG. 29 is a scheme illustrating the synthesis of compounds of the invention that target and degrade tau neurofibrillary tangle (NFT).

FIG. 30, comprising FIGS. 30A-30B, illustrates hydrophobic tagging of endogenous NFTs. FIG. 30A: ¹⁸F-labeled DDNP derivative used clinically for PET imaging of amyloid proteins including Aβ deposits and NFTs in AD and other tauopathies. Note distinct pattern for labeling of NFTs in FTDP17 brain. FIG. 30B: Incorporation of hydrophobic tags into DDNP core enables targeted degradation of pre-existing NFTs.

FIG. 31 is a scheme illustrating the synthesis of compounds of the invention that target and degrade tau neurofibrillary tangle (NFT).

### DETAILED DESCRIPTION OF THE INVENTION

The present invention relates to compounds that act as degraders of target proteins, wherein degradation is independent of the class of protein or its localization. The target protein considered within the invention comprises any posttranslational modified protein or intracellular protein. Compounds of the present invention may be used to treat disease states wherein protein degradation is a viable therapeutic approach. Diseases contemplated within the invention include cancer, wherein the target protein is hyperexpressed or degradation of the protein triggers cell apoptosis, or any sort of oxidative stress disease state.

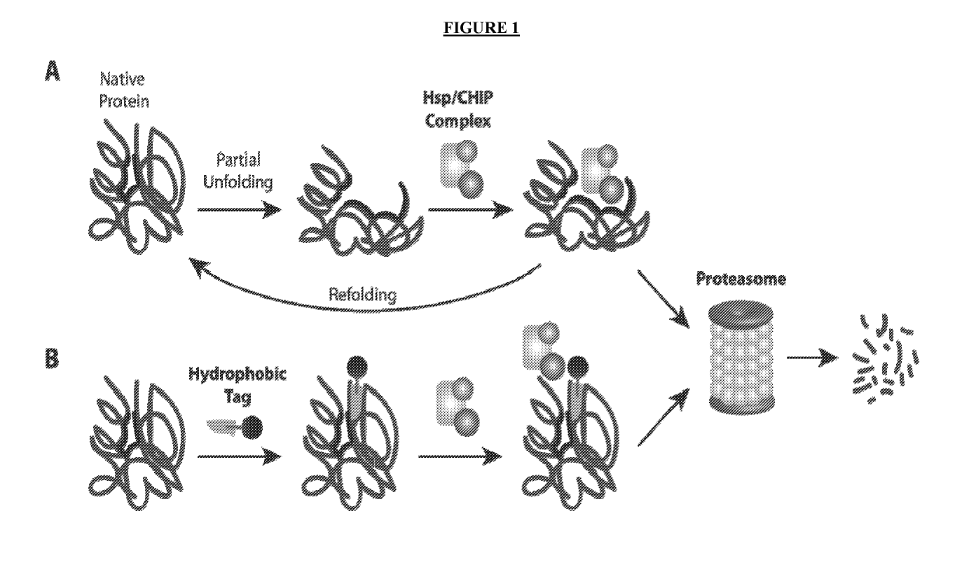

In one embodiment, the compound of the invention comprises a molecule comprising a linker that is covalently bound to a “greasy” or hydrophobic portion (wherein the hydrophobic portion is herein referred to as a degradation moiety or “degron”). The linker is selected so that it may be further covalently bound to a protein binding moiety (PBM), whereby the PBM and the degron are now part of the same molecule. The molecule comprising the degron and PBM may bind to the protein of choice, and the resulting tagged protein presents a hydrophobic surface. The cell then recognizes the tagged protein as being denatured and targets it for proteasomal degradation (FIG. 1B). This hydrophobic tagging strategy may be applied to any protein of choice, independently of its class or cellular location.

In another embodiment, the invention includes a high-throughput screening method to identify small molecules that are effective as therapeutic agents and have the ability to reach any gene product without requiring genetic modification of the target proteins.

### DEFINITIONS

As used herein, each of the following terms has the meaning associated with it in this section.

Unless defined otherwise, all technical and scientific terms used herein generally have the same meaning as commonly understood by one skilled in the art to which this invention belongs. Generally, the nomenclature used herein and the laboratory procedures in animal pharmacology, pharmaceutical science, separation science and organic chemistry are those known and commonly employed in the art.

In accordance with the present invention there may be employed conventional chemical synthetic methods, as well as molecular biology and biochemistry techniques within the skill of the art. Such techniques are well-known and are otherwise explained fully in the literature.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “DCM” refers to dichloromethane. As used herein, the term “DMF” refers to dimethyl formamide. As used herein, the term “RT” or “rt” refers to room temperature. As used herein, the term “NFT” refers to a neurofibrilar tangle.

As used herein, the term “associated” as applied to a protein in the context of a disease or disorder in a subject indicates that the presence or activity of the protein causes the disease or disorder in the subject, or the presence or activity of the protein prevents the subject from recovering from the disease or disorder, or the presence or activity of the protein antagonizes, hampers or prevents therapeutic interventions to treat or prevent the disease or disorder in the subject.

The term “compound,” as used herein, unless otherwise indicated, refers to any specific chemical compound disclosed herein. In one embodiment, the term also refer to stereoisomers and/or optical isomers (including racemic mixtures) or enantiomerically enriched mixtures of disclosed compounds. The term “compound” includes pharmaceutically acceptable salts thereof.

As used herein, a “solvate” of a compound refers to a complex between the compound and a finite number of solvent molecules. In one embodiment, the solvate is a solid isolated from solution by precipitation or crystallization. In another embodiment, the solvate is a hydrate.

As used herein, the term “RU59063” or “RU 59063” refers to (4-[3-(4-hydroxybutyl)-4,4-dimethyl-5-oxo-2-thioxoimidazolidin-1-yl]-2-(trifluoromethyl) benzonitrile, or a salt thereof.

As used herein, the term “bicalutamide” refers to N-[4-cyano-3-(trifluoromethyl)phenyl]-3-[(4-fluorophenyl)sulfonyl]-2-hydroxy-2-methylpropanamide, or a salt thereof.

As used herein, the term “geldanamycin” refers to (4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1]docosa-1(21),4,6,10,18-pentaen-9-yl carbamate, or a salt thereof. Geldanamycin is a benzoquinone ansamycin antibiotic that binds to Hsp90 (heat shock protein 90) and inhibits its function.

As used herein, the term “dimedone” refers to 5,5-dimethylcyclo-hexane-1,3-dione or a salt thereof.

As used herein, the term “SARD” refers to selective androgen receptor degrader.

As used herein, the term “heat shock protein 90 inhibitor” or “HSP 90 inhibitor” refers to a compound that inhibits heat shock protein 90 and facilitates and/or enhances proteosomal degradation and/or renaturation of target proteins. In one embodiment, a HSP 90 inhibitor is used in combination with a compound of the invention. In another embodiment, a HSP90 inhibitor enhances the pharmacological effect of a compound of the invention in an additive and/or synergistic manner. Exemplary HSP90 inhibitors include the ansamycin macrolactames, such as the quinones geldanamycin (GA), 17AAG/KOS953, 17-DMAG, CNF1010, tanespimycin and alvespimycin, KOS 1022, and the hydraquinones and their derivatives such as IPI540 (retaspimycin or 17-AAG hydroquinone); other derivatives such as KOSN 1559; the purines such as PU3, PUH58, PU24S, PU24FC1 and BIIB021; the pyrazole and isoxazole derivatives CCT018159, G3219, G3130, VER49009/CCT0129397 (analog of CCT018159), VER50589, STA-9090 and VER52296/NVP-AUY922; the dihydroindazolone derivatives SNS2112, SNX5422, the resorcylic inhibitors radicicol, cyclproparadicicol, KF 25706, KF 55823; coumarin inhibitors novobiocin (Nvb), chlorobiocin, coumermycin A1, compound A4, DHN2 and KU135, 4TCNA, 4TDHCNA and 4TTCQ, among others, including CUDC-305.

As used herein, the term “heat shock protein 70 modulator” or “HSP 70 modulator” refers to a compound that modulates (either through inhibition or through enhancement of activity as agonists) heat shock protein 70 and facilitates and/or enhances proteosomal degradation of target proteins pursuant to the present invention. Exemplary HSP70 modulators for use in the present invention include adenosine derived inhibitors of HSP70 as described by Williamson et al, 2009, J. Med. Chem. 52 (6):1510-1513, 2-phenylethynesulfonamide (PES) and geranylgeranylacetone, among others, including compounds as set forth in US Patent Publication No. 20070259820, all of which are incorporated by reference herein.

As used herein, the term “sulfenome” refers to a collection of proteins comprising sulfenic acid residues (which are most likely derived from oxidation of cysteinyl residues) found in a cell, usually under oxidative stress.

As used herein, the term “degron,” “degradation moiety,” “hydrophobic group,” or “hydrophobic moiety” refers to a protein degradation moiety (DM), which is defined as a hydrophobic moiety that can be further covalently bound to a linker. Once attached to a protein, the degron destabilizes the target protein, causing the target protein to be degraded in the cell (generally, through proteasomal degradation). The degron in combination with the linker group has a ClogP value of at least about 1.25, at least about 1.5, at least about 1.75, at least about 2.0, at least about 2.25, at least about 2.5, at least about 2.75, at least about 3.0, at least about 3.25, at least about 3.5, at least about 3.75, at least about 4.0, at least about 4.25, at least about 4.5, at least about 4.75, at least about 5.0, at least about 5.25, or at least about 5.5.

As used herein, the term “ClogP” is a value readily calculated using ClogP software, available from Biobyte, Inc., Claremont, Calif., USA and applied to any computer that utilizes Windows, linux or an Apple operating system. ClogP software is readily adaptable to a number of chemical programs including ChemDraw programs and related chemical structure drawing programs. The value ClogP assigns to the hydrophobicity of a chemical compound or group is based upon a determination of log P n-octanol/water (log P_{OW}), which is the log of the partition coefficient of a molecule or group in octanol and water. ClogP accurately estimates log P_{OW} numbers and provides a readout of a value readily applied to the present invention. Newer versions of ChemDraw software, available from CambridgeSoft, Inc., Cambridge, Mass., USA. incorporate the ability to interface with ClogP software and provide ClogP calculations, which may be readily accomplished by simply drawing a molecule and applying the ClogP calculation app from that software to the hydrophobic molecule or group to be utilized.

As used herein, the term “protein binding moiety” or “PBM” refers to a moiety that selectively binds to a protein. In one embodiment, the protein binding moiety can be linked to a degron through a linker. Within this embodiment, the protein binding moiety binds to a target protein thereto, placing the degradation moiety in proximity to the target protein and triggering degradation.

As used herein, the term “linker” refers to a chemical group ranging in length from 2 to 60 atoms that can be further covalently bound to the hydrophobic moiety useful within the invention. In another embodiment, the linker may be bound to the hydrophobic moiety at one end and to the protein binding moiety at the other end. The linker may be used to link the hydrophobic moiety to the protein binding moiety using conventional chemistry, for example, by reacting a nucleophilic group on the protein binding moiety (such as an alcohol, amine, sulfhydryl or hydroxyl group) with an electrophilic group (such as a carboxylic acid) on the linker to which the hydrophobic groups is attached, thus creating a covalent bond between the hydrophobic moiety and the protein binding moiety through the linker.

As used herein, the term “target protein” or “protein” refers to a protein targeted by compounds of the present invention, in particular, the protein binding moiety. A target protein is any protein involved in the metabolism or catabolism of a cell and/or organ of a subject, especially including proteins that modulate a disease state or condition to be treated with compounds of the present invention. Target proteins may also include proteins from microbes, such as bacteria, viruses, fungi and protozoa. In general, target proteins may include, for example, structural proteins; receptors; enzymes; cell surface proteins; proteins pertinent to the integrated function of a cell, including proteins involved in catalysis, aromatase activity, motor activity, helicase activity, metabolic processes (such as anabolism and catabolism), antioxidant activity, proteolysis, biosynthesis, kinase activity, oxidoreductase activity, transferase activity, hydrolase activity, lyase activity, isomerase activity, ligase activity, enzyme regulator activity, signal transducer activity, structural molecule activity, binding activity (to a protein, lipid or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity), pathogenesis, chaperone regulator activity, nucleic acid binding activity, transcription regulator activity, extracellular organization and biogenesis activity, and translation regulator activity. Target proteins may include proteins from eukaryotes and prokaryotes, including humans and other animals, microbes, plants and viruses, among numerous others.

Non-limiting examples of target proteins include B7, B7-1 and B7-2 (providing second signals to T cells), TINFR1m, TNFR2, NADPH oxidase, BclIBax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, carbonic anhydrase, chemokine receptors, JAW STAT, RXR and similar, HIV 1 protease, HIV 1 integrase, influenza neuramimidase, hepatitis B reverse transcriptase, sodium channel, protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, Ras1Raf1MEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, tumor associated protein (TMP), Bcr-Abl tyrosine kinase, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Additional target proteins include acetyl-CoA carboxylase, adenylosuccinate synthetase, protoporphyrinogen oxidase, and enolpyruvylshikimate-phosphate synthase. Exemplary target proteins include, for example, drug resistant and multiple drug resistance (MDR) proteins.

As used herein, the term “neoplasia” refers to the pathological process that results in the formation and growth of a neoplasm, i.e., an abnormal tissue that grows by cellular proliferation more rapidly than normal tissue and continues to grow after the stimuli that initiated the new growth cease. Neoplasia exhibits partial or complete lack of structural organization and functional coordination with the normal tissue, and usually forms a distinct mass of tissue that may be benign (benign tumor) or malignant (cancer).

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds of the present invention. A more complete list of cancers that may be treated using compounds of the present invention may be found at the website cancer dot gov/cancertopics/alphalist, relevant portions of which are incorporated by reference herein.

As used herein, the term “non-cancer control sample” as relating to a subject's tissue refers to a sample from the same tissue type, obtained from the patient, wherein the sample is known or found not to be afflicted with cancer. For example, a non-cancer control sample for a subject's lung tissue refers to a lung tissue sample obtained from the subject, wherein the sample is known or found not to be afflicted with cancer. “Non-cancer control sample” for a subject's tissue also refers to a reference sample from the same tissue type, obtained from another subject, wherein the sample is known or found not to be afflicted with cancer. “Non-cancer control sample” for a subject's tissue also refers to a standardized set of data (such as, but not limited to, identity and levels of gene expression, protein levels, pathways activated or deactivated etc), originally obtained from a sample of the same tissue type and thought or considered to be a representative depiction of the non-cancer status of that tissue.

As used herein, the term “hyperproliferative cell growth” refers to conditions of abnormal cell growth of a non-transformed cell often, of the skin, distinguishable from cancer. Examples of such conditions include, for example, skin disorders such as hyperkeratosis, including ichthyosis, keratoderma, lichen, planus and psoriasis, warts, including genital warts, blisters and any abnormal or undesired cellular proliferation.

The term “additional anticancer agent” is used to describe a compound that may be combined with one or more compounds of the invention in the treatment of cancer and include such compounds/agents as everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, pyro-Glu-His-Trp-Ser-Tyr-D-Ser(Bu t)-Leu-Arg-Pro-Azgly-NH₂. x(acetate) wherein x=1 to 2.4, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

As used herein, the term “oxidative stress disease” refers to a disease state and/or condition where an oxidative state of stress exists, characterized by the presence of sulfenome. The oxidative stress results in the formation of a number of oxidized derivatives within tissue and/or cells of a patient or subject, especially including sulfenyl acid derivatives of cysteine. These disease states and/or conditions include for example, cancer, hyperproliferative cell growth conditions, Parkinson's disease, Alzheimer's disease, atherosclerosis, heart failure, including congestive heart failure, myocardial infarction, schizophrenia, bipolar disorder, fragile X syndrome, sick cell disease, chronic fatigue syndrome, aging (including aging by induction of mitohormesis, diabetes (especially type I) and vascular disease. In one embodiment, the compounds of the invention are useful in treating oxidative stress diseases and/or conditions, including cancer, and ameliorating secondary disease states and conditions of oxidative stress diseases and/or conditions.

As used herein, a “subject” may be a human or non-human mammal or a bird. Non-human mammals include, for example, livestock and pets, such as ovine, bovine, porcine, canine, feline and murine mammals. Preferably, the subject is human.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

Diseases or disorders that may be treated using compounds of the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, coeliac disease, Charcot-Marie-Tooth disease, cystic fibrosis, duchenne muscular dystrophy, haemochromatosis, haemophilia, Klinefelter's syndrome, neurofibromatosis, phenylketonuria, polycystic kidney disease, (PKD1) or 4 (PKD2) Prader-Willi syndrome, sickle-cell disease, Tay-Sachs disease, and Turner syndrome.

Further disease states or conditions that may be treated by compounds of the present invention include Alzheimer's disease, amyotrophic lateral sclerosis (Lou Gehrig's disease), anorexia nervosa, anxiety disorder, atherosclerosis, attention deficit hyperactivity disorder, autism, bipolar disorder, chronic fatigue syndrome, chronic obstructive pulmonary disease, Crohn's disease, coronary heart disease, dementia, depression, diabetes mellitus type 1, diabetes mellitus type 2, epilepsy, Guillain-Barré syndrome, irritable bowel syndrome, lupus, metabolic syndrome, multiple sclerosis, myocardial infarction, obesity, obsessive-compulsive disorder, panic disorder, Parkinson's disease, psoriasis, rheumatoid arthritis, sarcoidosis, schizophrenia, stroke, thromboangiitis obliterans, Tourette syndrome, and vasculitis.

Additional disease states or conditions that may be treated by compounds of the present invention include aceruloplasminemia, achondrogenesis type II, achondroplasia, acrocephaly, Gaucher disease type 2, acute intermittent porphyria, Canavan disease, adenomatous Polyposis Coli, ALA dehydratase deficiency, adenylosuccinate lyase deficiency, adrenogenital syndrome, adrenoleukodystrophy, ALA-D porphyria, ALA dehydratase deficiency, alkaptonuria, Alexander disease, alkaptonuric ochronosis, alpha 1-antitrypsin deficiency, alpha-1 proteinase inhibitor, emphysema, amyotrophic lateral sclerosis, Alström syndrome, Alexander disease, Amelogenesis imperfecta, ALA dehydratase deficiency, Anderson-Fabry disease, androgen insensitivity syndrome, anemia, angiokeratoma corporis diffusum, angiomatosis retinae (von Hippel-Lindau disease), Apert syndrome, arachnodactyly (Marfan syndrome), Stickler syndrome, arthrochalasis multiplex congenital (Ehlers-Danlos syndrome#arthrochalasia type), ataxia telangiectasia, Rett syndrome, primary pulmonary hypertension, Sandhoff disease, neurofibromatosis type II, Beare-Stevenson cutis gyrata syndrome, mediterranean fever, familial, Benjamin syndrome, beta-thalassemia, bilateral acoustic neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndrome (Rubinstein-Taybi syndrome), bronze diabetes/bronzed cirrhosis (hemochromatosis), bulbospinal muscular atrophy (Kennedy's disease), Burger-Grutz syndrome (lipoprotein lipase deficiency), CGD chronic granulomatous disorder, campomelic dysplasia, biotinidase deficiency, cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, chondrodystrophy syndrome (achondroplasia), otospondylomegaepiphyseal dysplasia, Lesch-Nyhan syndrome, galactosemia, Ehlers-Danlos syndrome, thanatophoric dysplasia, Coffin-Lowry syndrome, Cockayne syndrome (familial adenomatous polyposis), congenital erythropoietic porphyria, congenital heart disease, methemoglobinemia/congenital methaemoglobinaemia, achondroplasia, X-linked sideroblastic anemia, connective tissue disease, conotruncal anomaly face syndrome, Cooley's Anemia (beta-thalassemia), copper storage disease (Wilson's disease), copper transport disease (Menkes disease), hereditary coproporphyria, Cowden syndrome, craniofacial dysarthrosis (Crouzon syndrome), Creutzfeldt-Jakob disease (prion disease), Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, degenerative nerve diseases including de Grouchy syndrome and Dejerine-Sottas syndrome, developmental disabilities, distal spinal muscular atrophy, type V, androgen insensitivity syndrome, diffuse globoid body sclerosis (Krabbe disease), Di George's syndrome, dihydrotestosterone receptor deficiency, androgen insensitivity syndrome, Down syndrome, dwarfism, erythropoietic protoporphyria, erythroid 5-aminolevulinate synthetase deficiency, erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), fibrocystic disease of the pancreas, fragile X syndrome, galactosemia, genetic brain disorders, giant cell hepatitis (neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), hyperandrogenism, hypochondroplasia, hypochromic anemia, immune system disorders, including X-linked severe combined immunodeficiency, Insley-Astley syndrome, Jackson-Weiss syndrome, Joubert syndrome, Lesch-Nyhan syndrome, Jackson-Weiss syndrome, kidney diseases, including hyperoxaluria, Klinefelter's syndrome, Kniest dysplasia, lacunar dementia, Langer-Saldino achondrogenesis, ataxia telangiectasia, Lynch syndrome, lysyl-hydroxylase deficiency, Machado-Joseph disease, metabolic disorders, Marfan syndrome, movement disorders, Mowat-Wilson syndrome, Muenke syndrome, multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford progeria syndrome), progressive chorea, chronic hereditary (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), recurrent polyserositis, retinal disorders, retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, skin pigmentation disorders, Smith-Lemli-Opitz syndrome, South-African genetic porphyria (variegate porphyria), infantile-onset ascending hereditary spastic paralysis, speech and communication disorders, sphingolipidosis, spinocerebellar ataxia, Stickler syndrome, stroke, androgen insensitivity syndrome, tetrahydrobiopterin deficiency, beta-thalassemia, thyroid disease, tomaculous neuropathy (hereditary neuropathy with liability to pressure palsies), Treacher Collins syndrome, triplo X syndrome (triple X syndrome), trisomy 21 (Down syndrome), trisomy X, VHL syndrome (von Hippel-Lindau disease), vision impairment and blindness (Alström syndrome), Vrolik disease, Waardenburg syndrome, Warburg Sjo Fledelius Syndrome, Weissenbacher-Zweymüller syndrome, Wolf-Hirschhorn syndrome, Wolff periodic disease, Weissenbacher-Zweymüller syndrome and Xeroderma pigmentosum, among others.

As used herein, the term “IC₅₀” refers to half maximal inhibitory concentration. As used herein, the term “DC₅₀” refers to half maximal degradation concentration.

As used herein, an “effective amount”, “therapeutically effective amount” or “pharmaceutically effective amount” of a compound is that amount of compound that is sufficient to provide a beneficial effect to the subject to which the compound is administered.

The terms “treat” “treating” and “treatment,” as used herein, means reducing the frequency or severity with which symptoms of a disease or condition are experienced by a subject by virtue of administering an agent or compound to the subject.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

In one aspect, the terms “co-administered” and “co-administration” as relating to a subject refer to administering to the subject a compound useful within the invention, or salt thereof, along with a compound that may also treat any of the diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

By the term “specifically bind” or “specifically binds,” as used herein, is meant that a first molecule preferentially binds to a second molecule (e.g., a particular receptor or enzyme), but does not necessarily bind only to that second molecule.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. The atom or group of atoms may be selected from the group consisting of hydroxyl, carboxyl, cyano (C≡N), nitro (NO₂), halogen (preferably, 1, 2 or 3 halogens, especially on an alkyl, especially a methyl group such as a trifluoromethyl), thiol, alkyl group (preferably, C₁-C₁₀, more preferably, C₁-C₆), alkoxy group (preferably, C₁-C₁₀ alkyl or aryl, including phenyl and substituted phenyl), ester (preferably, C₁-C₁₀ alkyl or aryl) including alkylene ester (such that attachment is on the alkylene group, rather than at the ester function which is preferably substituted with a C₁-C₁₀ alkyl or aryl group), thioether (preferably, C₁-C₁₀ alkyl or aryl), thioester (preferably, C₁-C₁₀ alkyl or aryl), (preferably, C₁-C₁₀ alkyl or aryl), halogen (F, Cl, Br, I), nitro or amine (including a five- or six-membered cyclic alkylene amine, further including a C₁-C₁₀ alkyl amine or C₁-C₁₀ dialkyl amine), amido, which is preferably substituted with one or two C₁-C₁₀ alkyl groups (including a carboxamide which is substituted with one or two C₁-C₁₀ alkyl groups), alkanol (preferably, C₁-C₁₀ alkyl or aryl), and alkanoic acid (preferably, C₁-C₁₀ alkyl or aryl). Preferably, the term “substituted” shall mean within its context of use alkyl, alkoxy, halogen, ester, keto, nitro, cyano and amine (especially including mono- or di-C₁-C₁₀ alkyl substituted amines). The term substituted may also include optionally substituted aryl and/or heterocyclic groups, including optionally substituted heteroaryl groups. Any substitutable position in a compound of the present invention may be substituted in the present invention, but preferably no more than 5, more preferably no more than 3 substituents are present on a single ring or ring system. The term substituted as used in the present invention also contemplates aryl (as otherwise described herein), including C₇-C₂₀ aralkyl substituents or heterocyclic, including heteroaryl substituents, each of which may be further substituted (including for example with C₁-C₁₂ alkylene groups). Preferably, the term “unsubstituted” shall often mean substituted with one or more H atoms, although the invention does contemplate fully saturated positions which may be construed as substituted when a normally unsaturated position is depicted.

For aryl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred.

Preferred substituents are those having hydrophobic characteristics as otherwise described herein. It is noted that the incorporation of a hydrophobic substituent onto an otherwise less hydrophobic or non-hydrophobic group may render the entire group hydrophobic as described for the present invention.

In the present invention, virtually any hydrophobic group, when combined with a linker group, having a calculated ClogP value of at least about 1.5 (as otherwise disclosed herein) may be used to facilitate the degradation of a target protein to which the protein binding moiety binds. Representative groups include optionally substituted hydrocarbyl groups containing at least three carbon atoms, such as optionally substituted C₃-C₃₀ alkyl, alkene or alkyne groups, including linear, branch-chained or cyclic (including bi-cyclo, adamantyl and fused ring groups) hydrocarbon groups, aryl groups, including aryl groups containing a single ring or 2 or more fused rings (e.g., two, three or four fused rings) such as optionally substituted phenyl groups, including optionally substituted naphthyl groups (including 1- or 2-naphthyl groups), optionally substituted anthracenyl, phenanthrenyl, and phenacenyl (chrysene) groups, optionally substituted diphenyl methyl or triphenyl methyl (trityl, methoxytrityl) groups, optionally substituted biphenyl groups, optionally substituted hydrophobic heterocyclic, including heteroaryl groups such as optionally substituted quinolinyl groups, among numerous others, including optionally substituted morpholine, optionally substituted piperidine or piperazine, as otherwise described herein. In one embodiment, useful hydrophobic moieties may have values of ClogP less than 1.5, but those moieties contain substantial steric bulk which compensates for the low levels of hydrophobicity. A substituent which is often used on aryl groups (e.g., phenyl, naphthyl) in the present invention is the borane nido-decaborane group (B₁₀H₁₄), which although is not a hydrophobic group per se, provides the favorable characteristics of a significant steric effect to enhance degradation of proteins in the present invention.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Disclosure

The invention relates to the hydrophobic tagging of target proteins for inducing intracellular protein degradation. The invention includes compounds that act as degraders of target proteins, wherein degradation is independent of the class of protein or its localization. The target protein considered within the invention comprises any posttranslational modified protein or intracellular protein. Compounds of the present invention may be used to treat disease states wherein protein degradation is a viable therapeutic approach, such as cancer or any oxidative stress disease state.

In one aspect, the compound of the invention comprises a molecule comprising a linker covalently bound to a “greasy” hydrophobic portion (herein referred to as a degradation moiety or “degron”). The linker is selected so that it may be further covalently bound to a protein binding moiety (PBM), whereby the PBM and the degron are now part of the same molecule. The molecule comprising the degron and PBM may bind to the protein of choice, and the resulting tagged protein presents a hydrophobic surface. The cell then recognizes the tagged protein as being denatured and targets it for proteasomal degradation (FIG. 1B). This hydrophobic tagging strategy may be applied to any protein of choice, independently of its class or cellular location.

In one non-limiting embodiment, the compounds of the invention target the androgen receptor (AR). A series of selective androgen receptor degraders (SARDs) based on the high affinity AR ligand RU 59063 were designed, wherein the AR binding group was connected via a short PEG linker to an adamantyl group. As demonstrated herein, the SARDs were effective in promoting AR degradation at low micromolar concentrations. In particular, a SARD comprising an ester bond on the linker group demonstrated a DC₅₀ (half maximum degradation concentration) value of 1 μM, while no degradation was detected for the parent RU 59063. SARDS comprising an amide bond or ether bond on the linker group also demonstrated good activity (DC₅₀s of about 2 μM each). Further, there was a strong correlation between AR levels and the levels of the key downstream biomarkers for prostate cancer, i.e., prostate specific antigen (PSA). Compounds lacking the hydrophobic group were active only at higher concentrations and did not achieve complete AR degradation at the concentrations assayed.

In one non-limiting embodiment, the compounds of the invention target the “sulfenome” by forming a covalent attachment to the sulfenic acid groups. The sulfenome is thus tagged with a hydrophobic tag, inducing its proteasomal degradation. In one embodiment, a dimedone (5,5-dimethylcyclo-hexane-1,3-dione) group recognizes the sulfenic acid modification on the target protein. Dimedones recognize and bind to sulfenic acid group, and no other nucleophilic or sulfur-containing functional groups in proteins such as amines, aldehydes, thiols or disulfide bonds. The sulfenic acid group reacts rapidly and specifically with the dimedone through condensation, with loss of water and irreversible formation of a thioether. In one embodiment, the dimedone group is labeled with a detection tag (such as a fluorescent molecule, biotin or rhodamine). These detection tags do not affect the specificity or reactivity of the dimedone group, and may enhance the cell permeability of the molecule.

### Compounds

The present invention includes a compound of formula (I), or a pharmaceutically acceptable salt, solvate or polymorph thereof:

L-DM (I), wherein:

DM is a protein degradation moiety (i.e., a “degron”), and L is a linker covalently bound to DM, such that L-DM has a total ClogP value of at least about 1.5, preferably at least about 2.0, preferably at least about 3.0 or higher.

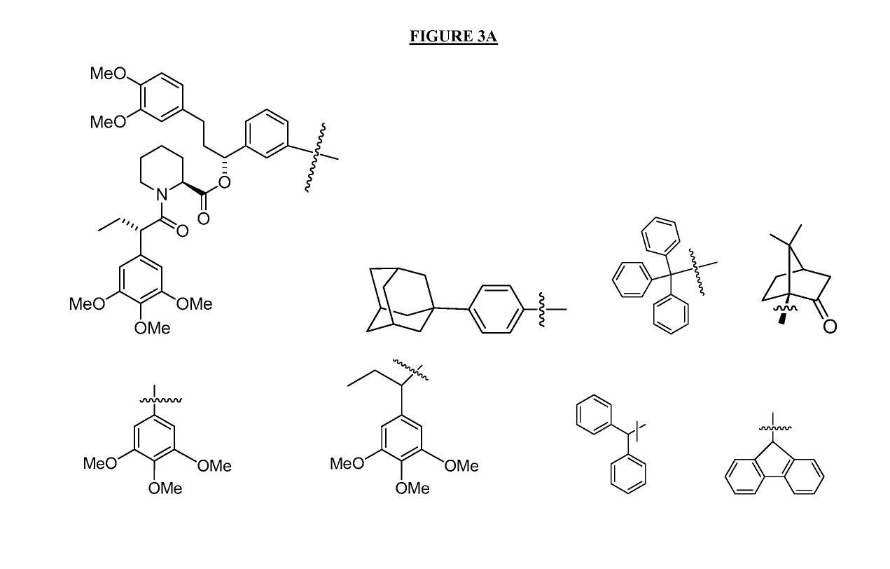

In one embodiment, DM is selected from the groups illustrated in FIG. 3. In another embodiment, DM does not comprise an optionally substituted argininyl group, an optionally substituted guanidine group, or a trisubstituted pyrrolidine group comprising two keto, thioketo, sulfoxide and/or sulfone groups bonded to the nitrogen atom of the pyrrolidine ring and the carbon atom alpha to the nitrogen of the pyrrolidine ring.

In one embodiment, L comprises at least 2 atoms in length. In another embodiment, L ranges in length from 2 to 60 atoms. In yet another embodiment, L ranges in length from 2 to 30 atoms. In yet another embodiment, L ranges in length from 2 to 8 atoms. In yet another embodiment, L ranges in length from 2 to 6 atoms.

In one embodiment, L comprises ethylene oxide groups ranging in size from about 2 to about 15, about 1 to about 10 or about 2 to about 8 ethylene oxide groups within the linker. Without wishing to be limited by theory, linkers comprising ethylene oxide groups may provide favorable bioavailability and pharmacokinetic attributes and readily enter cells where they may degrade target proteins.

In one embodiment, L comprises a group of formula (II):

—[Z—X—Y^{R}]— (II)

wherein Z links a protein binding moiety (PBM) to X; X links Z to group Y^{R}; and Y^{R} links to DM. In another embodiment, Y^{R} and DM share at least one common atom or group (for example, Y^{R} and DM form a cyclic structure together).

In one embodiment, Z and Y^{R} are independently a bond, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, —(CH₂)ᵢ—C≡C—, or —Y—C(O)—Y—, wherein:

R^{N} is H, C₁-C₃ alkyl or hydroxylated C₁-C₃ alkyl;

each occurrence of Y is independently a bond, O, S, —N(R^{N})—, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, or —(CH₂)ᵢ—C≡C—;

XY is —C(O)NH—, —NHC(O), —OC(O)NH—, —NHC(O)O—, —C(O)O—, —OC(O)—, —C(O)S—, or —SC(O)—; and,

each occurrence of i is independently an integer ranging from 0 to 100.

In one embodiment, each occurrence of i is independently an integer ranging from 1 to 75. In yet another embodiment, each occurrence of i is independently an integer ranging from 1 to 60. In yet another embodiment, each occurrence of i is independently an integer ranging from 1 to 55. In yet another embodiment, each occurrence of i is independently an integer ranging from 1 to 50. In yet another embodiment, each occurrence of i is independently an integer ranging from 1 to 45. In yet another embodiment, each occurrence of i is independently an integer ranging from 1 to 40. In yet another embodiment, each occurrence of i is independently an integer ranging from 2 to 35. In yet another embodiment, each occurrence of i is independently an integer ranging from 3 to 30. In yet another embodiment, each occurrence of i is independently an integer ranging from 1 to 15. In yet another embodiment, each occurrence of i is independently an integer ranging from 1 to 10. In yet another embodiment, each occurrence of i is independently an integer ranging from 1 to 8. In yet another embodiment, each occurrence of i is independently 1, 2, 3, 4, 5 or 6.

In one embodiment, X is -(D-CON-D)ᵢ-, wherein

each occurrence of D is independently a bond, —(CH₂)ᵢ—Y—C(═O)—Y—(CH₂)ᵢ—, —(CH₂)ᵢ— or —[(CH₂)ᵢ—X¹]ᵢ—;

each occurrence of i is independently an integer ranging from 0 to 100;

X¹ is O, S or N—R⁴;

each occurrence of Y is independently a bond, O, S, —N(R^{N})—, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, or —(CH₂)ᵢ—C≡C—;

each occurrence of R^{N} is independently H, C₁-C₃ alkyl or hydroxylated C₁-C₃ alkyl;

XY is —C(O)NH—, —NHC(O), —OC(O)NH—, —NHC(O)O—, —C(O)O—, —OC(O)—, —C(O)S—, or —SC(O);

CON is a bond, —C(O)NH—, —NH(CO)—, —X²—, —X³—C(O)—X³—,

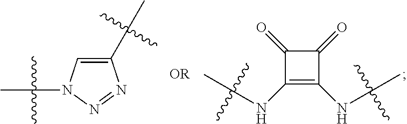 [SMILES: *c1cn(*)nn1 ; *Nc1c(N*)c(=O)c1=O]

X² is —O—, —S—, —N(R⁴)—, —S(O)—, —S(O)₂—, —S(O)₂O—, —OS(O)₂, or OS(O)₂O;

X³ is O, S, or NR⁴; and,

R⁴ is H or C₁-C₃ alkyl group.

In one embodiment, CON is —C(O)NH—, —NH(CO)—, or

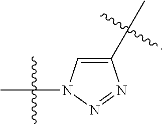 [SMILES: *c1cn(*)nn1]

In another embodiment, R¹ and R² are H.

In one embodiment, L is further covalently bound to a protein binding moiety (PBM) (i.e., a group that binds to any protein targeted for degradation). In another embodiment, the compound of formula (I) is the compound of formula (Ia) or a pharmaceutically acceptable salt, solvate or polymorph thereof:

PBM-L-DM (Ia),

wherein PBM is a protein binding moiety. In one embodiment, the protein targeted by the PBM comprises an androgen receptor, a neurofibrillary tangle, or a sulfenic acid-comprising protein (i.e., the protein comprises sulfenic acid groups within the polypeptide sequence.

In one embodiment, the PBM binds to and forms a covalent linkage to the target protein.

In one embodiment, PBM is selected from the group consisting of:

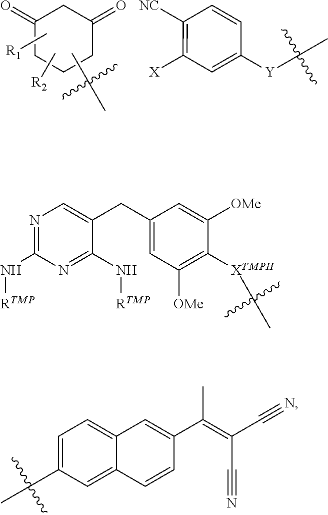

wherein:

each occurrence of R¹ and R² is independently selected from the group consisting of H, substituted C₁-C₆ alkyl, substituted C₂-C₆ alkynyl, —C(O)(C₁-C₆ alkyl), —NO₂, —CN, —F, —Cl, —Br, —I, —CF₃, —C(O)CF₃ and —C≡C—Rₐ, wherein each alkyl or alkynyl group is optionally and independently substituted with 1-6 electron withdrawing groups;

Rₐ is H or C₁-C₆ alkyl;

X is NO₂, CN, F, Cl, Br, I, —C≡C—Rₐ, CF₃, or —C(O)CF₃;

Y is a bond,

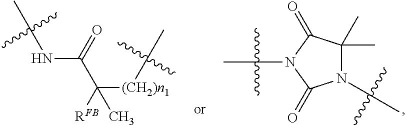

wherein R^{FB} is H or OH, and n₁ is 0, 1, 2, or 3;

each occurrence of R^{TMP} is independently H, C₁-C₂₀ alkyl or C₁-C₂₀ acyl;

X^{TMP} is O, S, S(O)₂, CH₂ or NR^{FB1};

each occurrence of R^{FB1} is independently H or a C₁-C₃ alkyl group substituted with 1-3 hydroxyl groups; and,

each occurrence of n₂ is independently 0, 1, 2, or 3.

In one embodiment, the electron withdrawing group comprises NO₂, CN, F, Cl, Br or C(O)CH₃. In another embodiment, R¹ and R² are H.

Non-limiting examples of protein binding moieties (PBM) useful within the invention include the following:

### I. Heat Shock Protein 90 (HSP90) Inhibitors:

HSP90 inhibitors as used herein include, but are not limited to:
HSP90 inhibitors identified in Vallee et al., 2011, J. Med. Chem. 54:7206, including N-[4-(3H-imidazo[4,5-C]pyridin-2-yl)-9H-fluoren-9-yl]-succinamide:

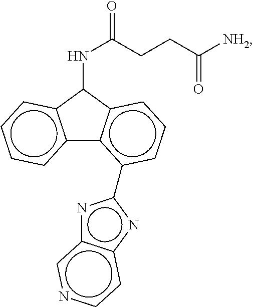

wherein L may be attached via the terminal amide group;
the HSP90 inhibitor p54 (modified): 8-[(2,4-dimethylphenyl)sulfanyl]-3-pent-4-yn-1-yl-3H-purin-6-amine)

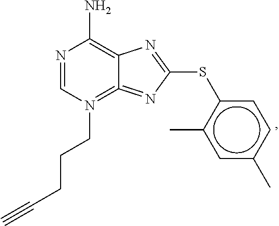

wherein L may be attached via the terminal acetylene group;
HSP90 inhibitors (modified) identified in Brough et al., 2008, J. Med. Chem. 51:196, including the compound 5-(2,4-dihydroxy-5-isopropylphenyl)-N-ethyl-4-(4-(morpholinomethyl)phenyl)isoxazole-3-carboxamide:

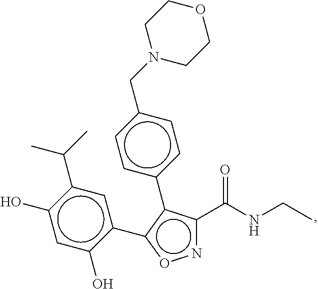

wherein L may be attached via the amide group (by forming a chemical bond with the amide N or the alkyl group on the amide N);
HSP90 inhibitors (modified) identified in Wright et al., 2004, Chem. Biol. 11(6):775-85, including the HSP90 inhibitor PU3 having the structure:

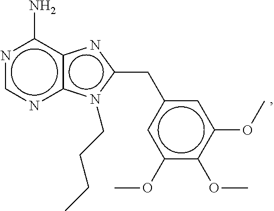

wherein L may be attached through the butyl group; and
HSP90 inhibitor geldanamycin ((4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1] (derivatized) or any of its derivatives (e.g., 17-alkylamino-17-desmethoxy-geldanamycin (“17-AAG”) or 17-(2-dimethylaminoethyl)amino-17-desmethoxygeldanamycin (“17-DMAG”)) (derivatized, where L may be attached through the amide group).

### II. Kinase and Phosphatase Inhibitors:

Kinase inhibitors contemplated within the invention include, but are not limited to:

Erlotinib derivative tyrosine kinase inhibitor:

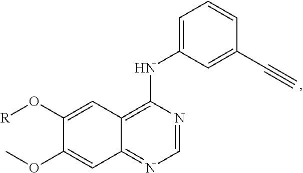

wherein R is L-DM;
Kinase inhibitor sunitanib (derivatized):

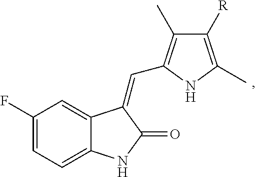

wherein R is L-DM;
Kinase inhibitor sorafenib (derivatized)

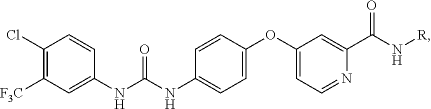

wherein R is L-DM;
Kinase inhibitor desatinib (derivatized)

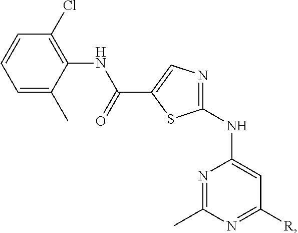

wherein R is L-DM;
Kinase inhibitor lapatinib (derivatized):

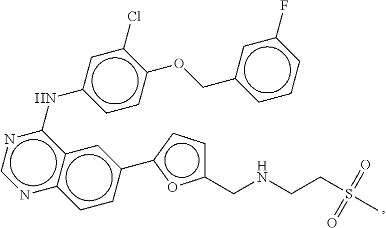

wherein L may be attached to the terminal methyl of the sulfonyl methyl group;
Kinase inhibitor U09-CX-5279 or 3-(cyclopropylamino)-5-((3-(trifluoromethyl)phenyl)amino)pyrimido[4,5-c]quinoline-8-carboxylic acid (derivatized):

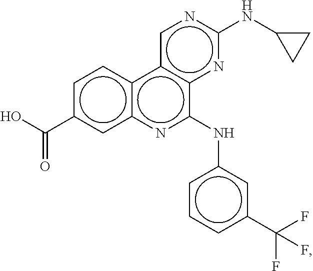

wherein L may be attached through the disubstituted aniline group, carboxylic acid, amine alpha to cyclopropyl group, or cyclopropyl group;
Kinase inhibitors identified in Millan et al., 2011, J. Med. Chem. 54:7797, including the kinase inhibitors Y1W (1-(3-(tert-butyl)-1-phenyl-1H-pyrazol-5-yl)-3-(2-((3-isopropyl-[1,2,4]triazolo[4,3-a]pyridin-6-yl)thio)benzyl)urea) and Y1X (or 1-ethyl-3-(2-((3-isopropyl-[1,2,4]triazolo[4,3-a]pyridin-6-yl)thio)benzyl)urea):

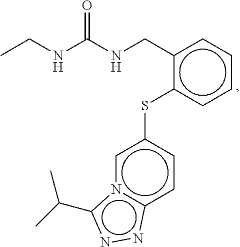

wherein L may be attached through the isopropyl group;

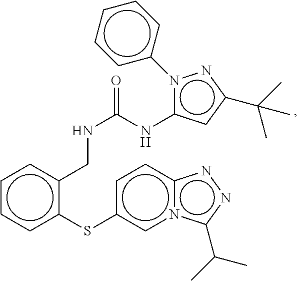

wherein L may be attached through the propyl group or the butyl group;
Kinase inhibitors identified in Schenkel et al, 2011, J. Med. Chem. 54(24):8440-50, including the compounds 6TP (4-amino-2-(4-(N-(tert-butyl)sulfamoyl)phenyl)-N-methylthieno[3,2-c]pyridine-7-carboxamide) and 0TP (4-amino-N-methyl-2-(4-morpholinophenyl)thieno[3,2-c]pyridine-7-carboxamide):

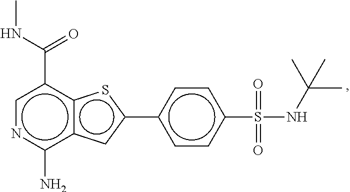

wherein L may be attached through the terminal methyl group bound to amide moiety;

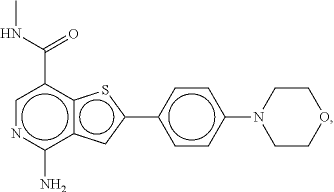

wherein L may be attached through the terminal methyl group bound to the amide moiety;
Kinase inhibitors identified in Van Eis et al., 2011, Bioorg. Med. Chem. Lett. 21(24):7367-72, including the kinase inhibitor 07U (2-methyl-N1-(3-(pyridin-4-yl)-2,6-naphthyridin-1-yl)propane-1,2-diamine):

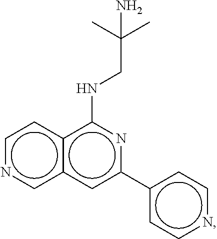

wherein L may be attached through the secondary amino or terminal amino group;
Kinase inhibitors identified in Lountos et al., 2011, J. Struct. Biol. 176:292, including the kinase inhibitor YCF ((E)-N-hydroxy-2-(1-(4-(3-(4-((E)-1-(2-((E)-N′-hydroxycarbamimidoyl)hydrazono)ethyl)phenyl)ureido)phenyl)ethylidene) hydrazinecarboximidamide):

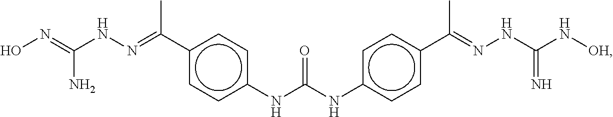

wherein L may be attached through either of the terminal hydroxyl groups;
Kinase inhibitors identified in Lountos et al., 2011, J. Struct. Biol. 176:292, including the kinase inhibitors XK9 ((E)-N-(4-(1-(2-(N-hydroxycarbamimidoyl) hydrazono)ethyl)phenyl)-7-nitro-1H-indole-2-carboxamide) and NXP ((E)-N-(4-(1-(2-carbamimidoylhydrazono)ethyl)phenyl)-1H-indole-3-carboxamide) (derivatized):

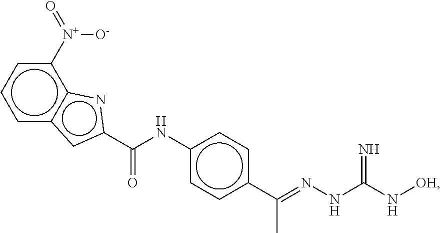

wherein L may be attached through the terminal hydroxyl group;

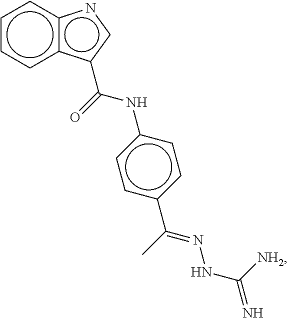

wherein L may be attached through the terminal amino group;
Kinase inhibitor afatinib (derivatized) (N-[4-[(3-Chloro-4-fluorophenyl)amino]-7-[[(3S)-tetrahydro-3-furanyl]oxy]-6-quinazolinyl]-4(dimethylamino)-2-butenamide):

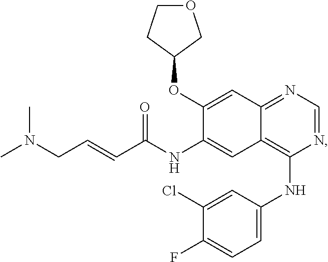 [SMILES: CN(C)C/C=C/C(=O)Nc1cc2c(Nc3ccc(F)c(Cl)c3)ncnc2cc1O[C@H]1CCOC1]

wherein L may be attached through the aliphatic amino group;
Kinase inhibitor fostamatinib (derivatized) ([6-({5-fluoro-2-[(3,4,5-trimethoxyphenyl)amino]pyrimidin-4-yl}amino)-2,2-dimethyl-3-oxo-2,3-dihydro-4H-pyrido[3,2-b]-1,4-oxazin-4-yl]methyl disodium phosphate hexahydrate):

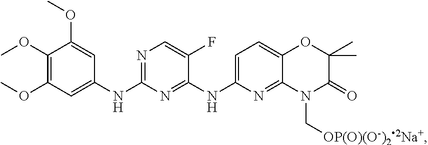

wherein L may be attached through one of the methoxy groups;
Kinase inhibitor gefitinib (derivatized) (N-(3-chloro-4-fluoro-phenyl)-7-methoxy-6-(3-morpholin-4-ylpropoxy)quinazolin-4-amine):

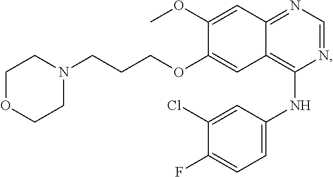 [SMILES: COc1cc2ncnc(Nc3ccc(F)c(Cl)c3)c2cc1OCCCN1CCOCC1]

wherein L may be attached through the methoxy group;

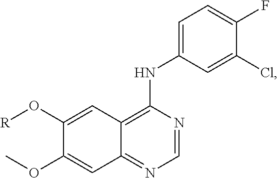

wherein R is L-DM;
Kinase inhibitor lenvatinib (derivatized) (4-[3-chloro-4-(cyclopropylcarbamoyl amino)phenoxy]-7-methoxy-quinoline-6-carboxamide):

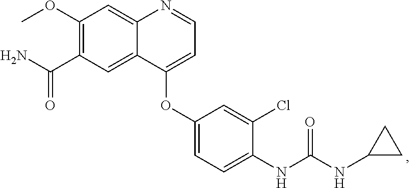 [SMILES: COc1cc2nccc(Oc3ccc(NC(=O)NC4CC4)c(Cl)c3)c2cc1C(N)=O]

wherein L may be attached through the cyclopropyl group;
Kinase inhibitor vandetanib (derivatized) (N-(4-bromo-2-fluorophenyl)-6-methoxy-7-[(1-methylpiperidin-4-yl)methoxy]quinazolin-4-amine):

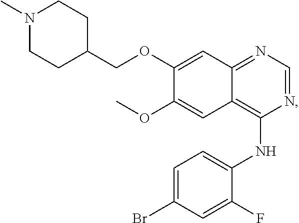 [SMILES: COc1cc2c(Nc3ccc(Br)cc3F)ncnc2cc1OCC1CCN(C)CC1]

wherein L may be attached through the methoxy or hydroxyl group;
Kinase inhibitor vemurafenib (derivatized) (propane-1-sulfonic acid {3-[5-(4-chlorophenyl)-1H-pyrrolo[2,3-b]pyridine-3-carbonyl]-2,4-difluoro-phenyl}-amide):

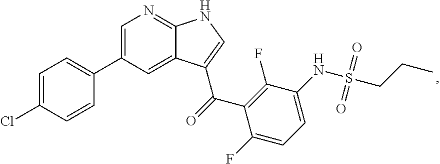 [SMILES: CCCS(=O)(=O)Nc1ccc(F)c(C(=O)c2c[nH]c3ncc(-c4ccc(Cl)cc4)cc23)c1F]

wherein L may be attached through the sulfonyl propyl group;
Kinase inhibitor Gleevec (derivatized):

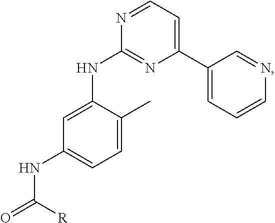

wherein L may be attached through the amide group or the aniline amino group;
Kinase inhibitor pazopanib (derivatized) (VEGFR3 inhibitor):

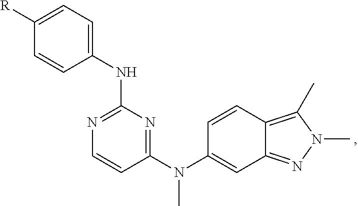

wherein R is L-DM, or L may be attached through the aniline amino group;
Aurora Kinase inhibitor AT-9283 (Derivatized):

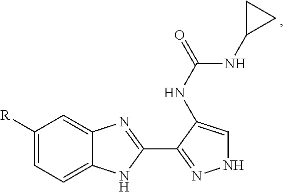

wherein R is L-DM;
ALK Kinase inhibitor TAE684 (derivatized):

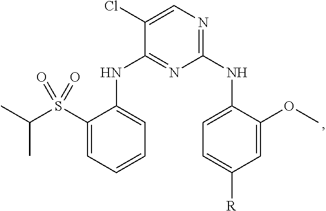

wherein R is L-DM;
Abl kinase inhibitor nilotanib (derivatized):

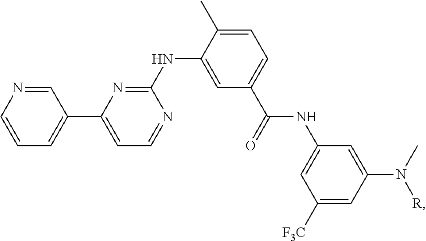

wherein R is L-DM; or L may be attached through the aniline amino group;
JAK2 kinase inhibitor NVP-BSK805 (derivatized):

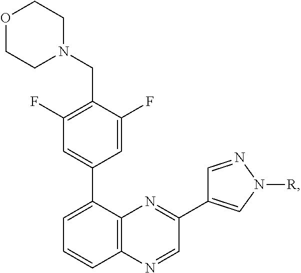

wherein R is L-DM; or L may be attached through the phenyl group;
Alk kinase inhibitor crizotinib:

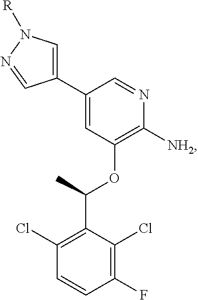

wherein R is L-DM or L may be attached through the amino group;
FMS kinase inhibitor (derivatized) inhibitor:

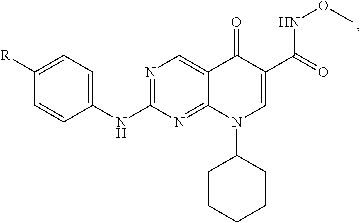

wherein R is L-DM;
Met kinase inhibitor Foretinib (derivatized):

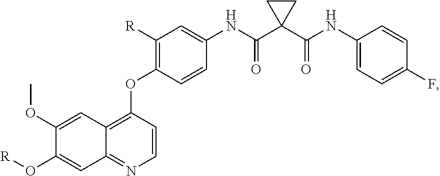

wherein either R is L-DM;
Allosteric protein tyrosine phosphatase inhibitor PTP1B (derivatized):

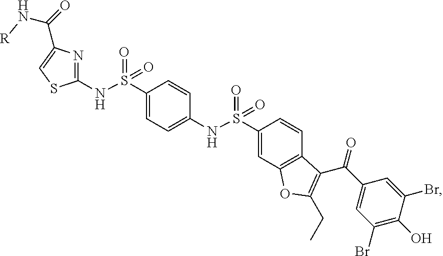

wherein R is L-DM;
Inhibitor of SHP-2 Domain of Tyrosine Phosphatase (derivatized):

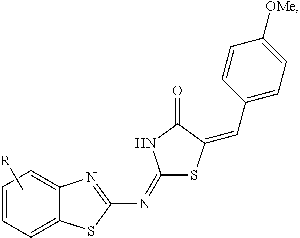

wherein R is L-DM;
BRAF (BRAF^{V600E})/MEK inhibitor (derivatized):

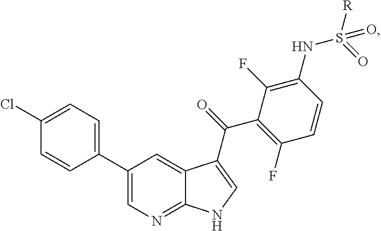

wherein R is L-DM;
ABL inhibitor (derivatized) tyrosine kinase:

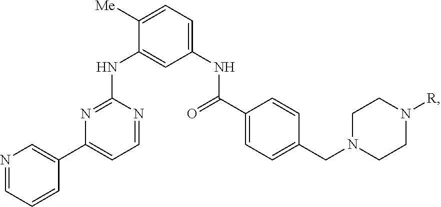

wherein R is L-DM;

### III. MDM2 Inhibitors:

MDM2 inhibitors as used herein include, but are not limited to:
MDM2 inhibitors identified in Vassilev et al., 2004, Science 303:844-848, and Schneekloth et al., 2008, Bioorg. Med. Chem. Lett. 18:5904-5908, including the compounds nutlin-3, nutlin-2, and nutlin-1 (derivatized) as described below, as well as all derivatives and analogs thereof:

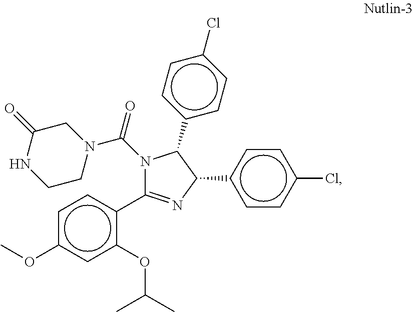

wherein L may be attached to the methoxy group or hydroxyl group;

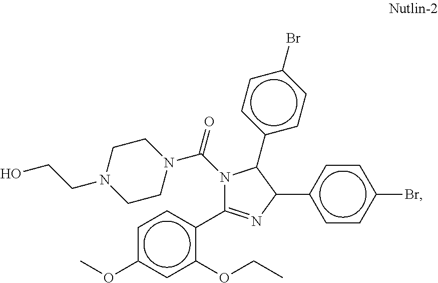

wherein L may be attached to the methoxy group or hydroxyl group;

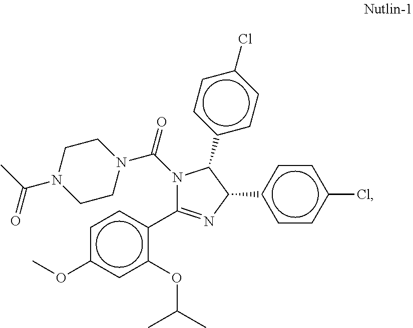

wherein L may be attached to the methoxy group or hydroxyl group;
Trans-4-iodo-4′-boranyl-chalcone:

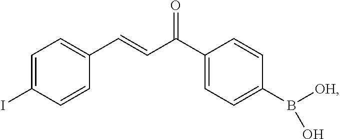 [SMILES: O=C(/C=C/c1ccc(I)cc1)c1ccc(B(O)O)cc1]

wherein L may be attached to the hydroxyl group)

### IV. Compounds Targeting Human BET Bromodomain-Containing Proteins:

Compounds targeting human BET bromodomain-containing proteins include, but are not limited to the compounds associated with the targets as described below, where “R” designates a potential site for linker attachment (R is L-DM):
Protein targets: Brd2, Brd3, Brd4

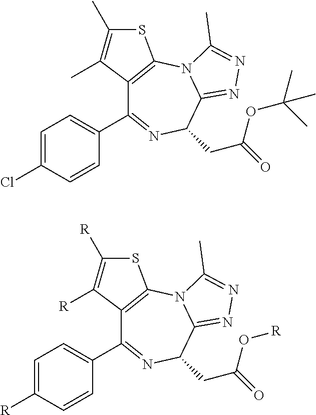

Filippakopoulos et al., Selective inhibition of BET bromodomains. Nature (2010)

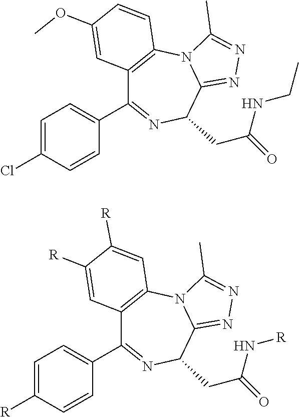

I-BET, Nicodeme et al., 2010, Suppression of inflammation by a synthetic histone mimic, Nature; Chung et al., 2011, Discovery and characterization of small molecule inhibitors of the BET family bromodomains, J. Med. Chem.

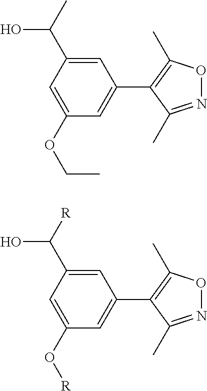

Hewings et al., 2011, 3,5-Dimethylisoxazoles act as acetyl-lysine-mimetic bromodomain ligands. J. Med. Chem. 54 (191:6761-70

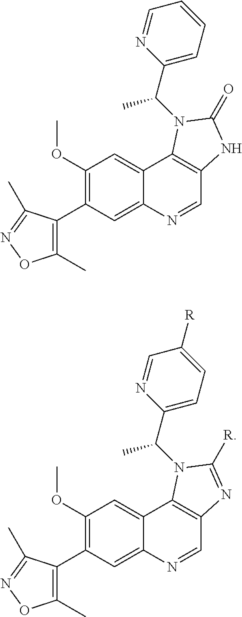

### V. HDAC Inhibitors:

HDAC Inhibitors (derivatized) include, but are not limited to:

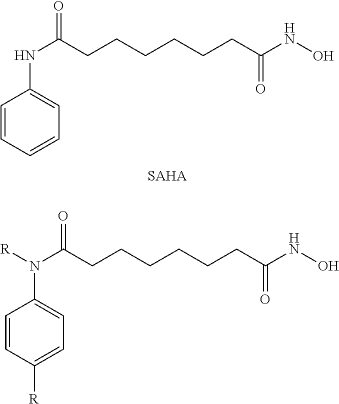

Finnin et al., 1999, Nature 401:188-193; wherein R is L-DM.
Compounds as defined by formula (I) of PCT WO0222577, wherein L may be attached through the hydroxyl group);

### VI. Human Lysine Methyltransferase Inhibitors:

Human Lysine Methyltransferase inhibitors include, but are not limited to:

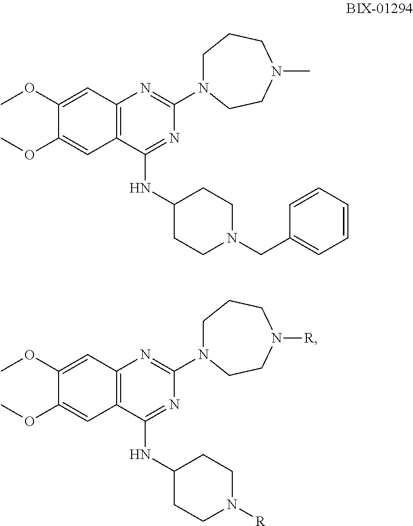

wherein R is L-DM (Chang et al., 2009, Nat. Struct. Mol. Biol. 16(3):312-7);

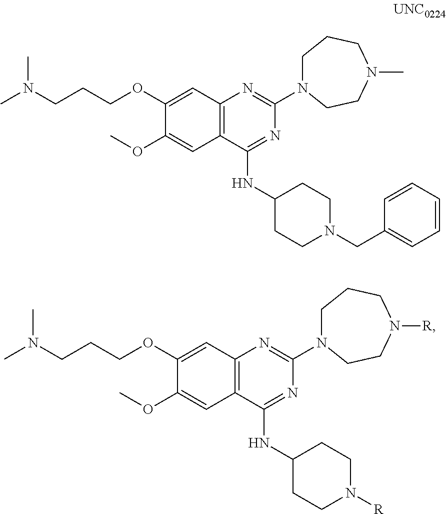

wherein R is L-DM (Liu et al., 2009, J. Med. Chem. 52(24):7950-3);
Azacitidine (derivatized) (4-amino-1-β-D-ribofuranosyl-1,3,5-triazin-2(1H)-one), wherein L may be attached through the hydroxy or amino groups; and
Decitabine (derivatized) (4-amino-1-(2-deoxy-β-D-erythro-pentofuranosyl)-1,3,5-triazin-2(1H)-one), wherein L may be attached through the hydroxy or amino groups; and

### VII. Angiogenesis Inhibitors:

Angiogenesis inhibitors include, but are not limited to:
GA-1 (derivatized) and derivatives and analogs thereof, having the structure(s) and being derivatized with linkers as described in Sakamoto et al., 2003, Mol. Cell Proteomics 2(12):1350-8;
Estradiol (derivatized), which may be bound to L as generally described in Rodriguez-Gonzalez et al., 2008, Oncogene 27:7201-7211;
Estradiol, testosterone (derivatized) and related derivatives, including but not limited to DHT and derivatives and analogs thereof, having the structure(s) and being derivatized with linkers as generally described in Sakamoto et al., 2003, Mol. Cell Proteomics 2(12):1350-8; and
Ovalicin, fumagillin (derivatized), and derivatives and analogs thereof, having the structure(s) and being derivatized with linkers as generally described in Sakamoto et al., 2001, Proc. Natl. Acad. Sci. 98(15):8554-9 and U.S. Pat. No. 7,208,157.

### VIII. Immunosuppressive Compounds:

Immunosuppressive compounds include, but are not limited to:
AP21998 (derivatized), having the structure(s) and being derivatized with linkers as generally described in Schneekloth et al., 2004, J. Am. Chem. Soc. 126:3748-3754;
Glucocorticoids (e.g., hydrocortisone, prednisone, prednisolone, and methylprednisolone) (derivatized where L may be bound to any of the hydroxyl groups, for example) and beclometasone dipropionate (derivatized where a linker may be bound to a proprionate group, for example);
Methotrexate (derivatized where L may be bound to either of the terminal hydroxyl groups, for example);
Ciclosporin (derivatized where L may be bound to at any of the butyl groups, for example);
Tacrolimus (FK-506) and rapamycin (derivatized where L may be bound to at one of the methoxy groups, for example);
Actinomycins (d derivatized where L may be bound to at one of the isopropyl groups, for example).

### IX. Compounds Targeting the Aryl Hydrocarbon Receptor (AHR):

Compounds targeting the aryl hydrocarbon receptor (AHR) include, but are not limited to:
Apigenin (derivatized in a way which may bind to L as generally illustrated in Lee et al., 2007, ChemBioChem 8(17):2058-2062);
SR1 and LGC006 (derivatized, as described in Boitano et al., 2010, Science 329(5997):1345-1348.

### X. Compounds Targeting RAF Receptor (Kinase):

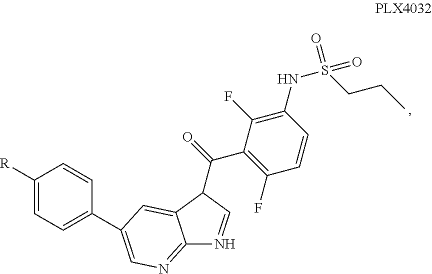 [0286] wherein R is L-DM.

### XI. Compounds Targeting FKBP

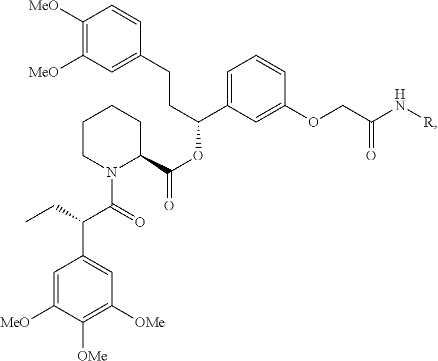 [0288] wherein R is L-DM.

### XII. Compounds Targeting Androgen Receptor (AR)

RU59063 ligand (derivatized):

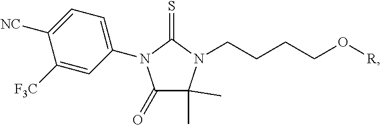

wherein R is L-DM;
SARM ligand (derivatized):

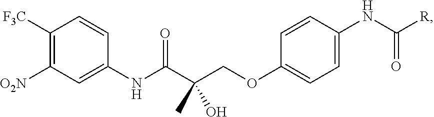

wherein R is L-DM;
Androgen receptor ligand DHT (derivatized):

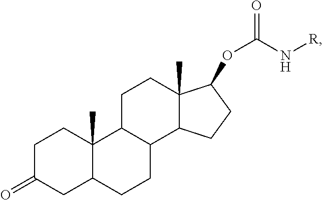

wherein R is L-DM;

### XIII. Compounds Targeting Estrogen Receptor (ER) ICI-182780

Estrogen Receptor Ligand

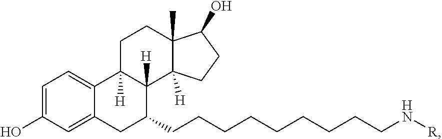

wherein R is L-DM.

### XIV. Compounds Targeting Thyroid Hormone Receptor (TR)

Thyroid hormone receptor ligand (derivatized):

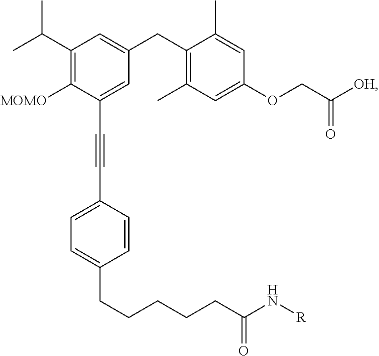

wherein R is L-DM; and MOMO indicates a methoxymethoxy group).

### XV. Compounds Targeting HIV Protease

Inhibitor of HIV Protease (derivatized):

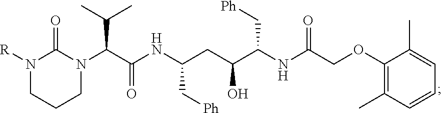

wherein R is L-DM (J. Med. Chem. 2010, 53:521-538).
Inhibitor of HIV protease:

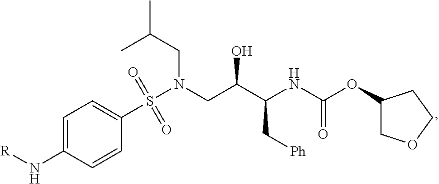

wherein R is L-DM (J. Med. Chem. 2010, 53:521-538).

### XVI. Compounds Targeting HIV Integrase:

Inhibitor of HIV integrase (derivatized):

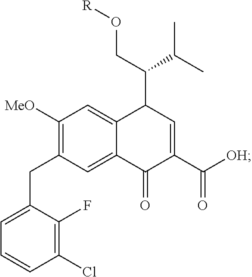

wherein R is L-DM (J. Med. Chem. 2010, 53:6466).
Inhibitor of HIV integrase (derivatized)

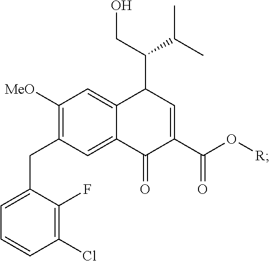

wherein R is L-DM (J. Med. Chem. 2010, 53:6466).

### XVII. Compounds Targeting HCV Protease

Inhibitors of HCV protease (derivatized):

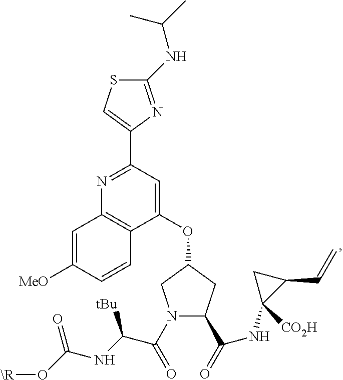

wherein R is L-DM.

### XVIII. Compounds Targeting Acyl-protein Thioesterase-1 and -2 (APT1 and APT2)

Inhibitor of APT1 and APT2 (derivatized):

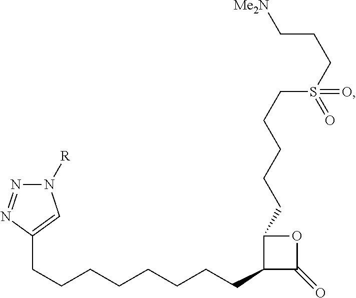

wherein R is L-DM (Angew. Chem. Int. Ed. 2011, 50:9838-9842).

Non-limiting examples of compounds that target the DHFR enzyme include the following compounds or a salt thereof:

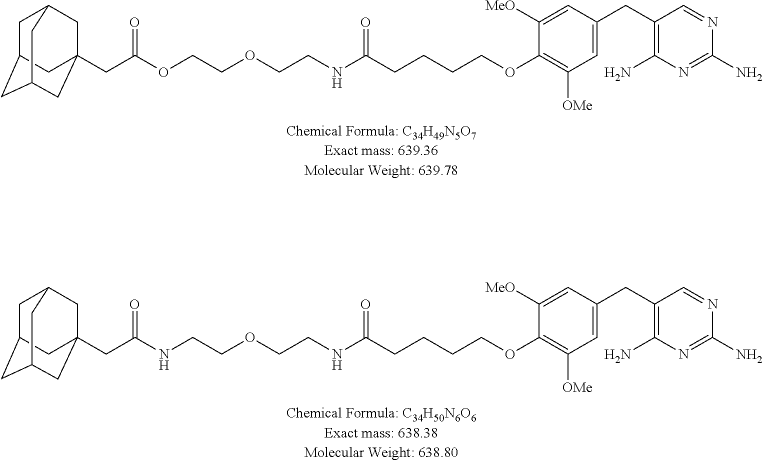 [SMILES: COc1cc(Cc2cnc(N)nc2N)cc(OC)c1OCCCCC(=O)NCCOCCOC(=O)CC12CC3CC(CC(C3)C1)C2 ; COc1cc(Cc2cnc(N)nc2N)cc(OC)c1OCCCCC(=O)NCCOCCNC(=O)CC12CC3CC(CC(C3)C1)C2]

Non-limiting examples of compounds that target the retinoid-related orphan receptor α (RORα) include the following compounds or a salt thereof:

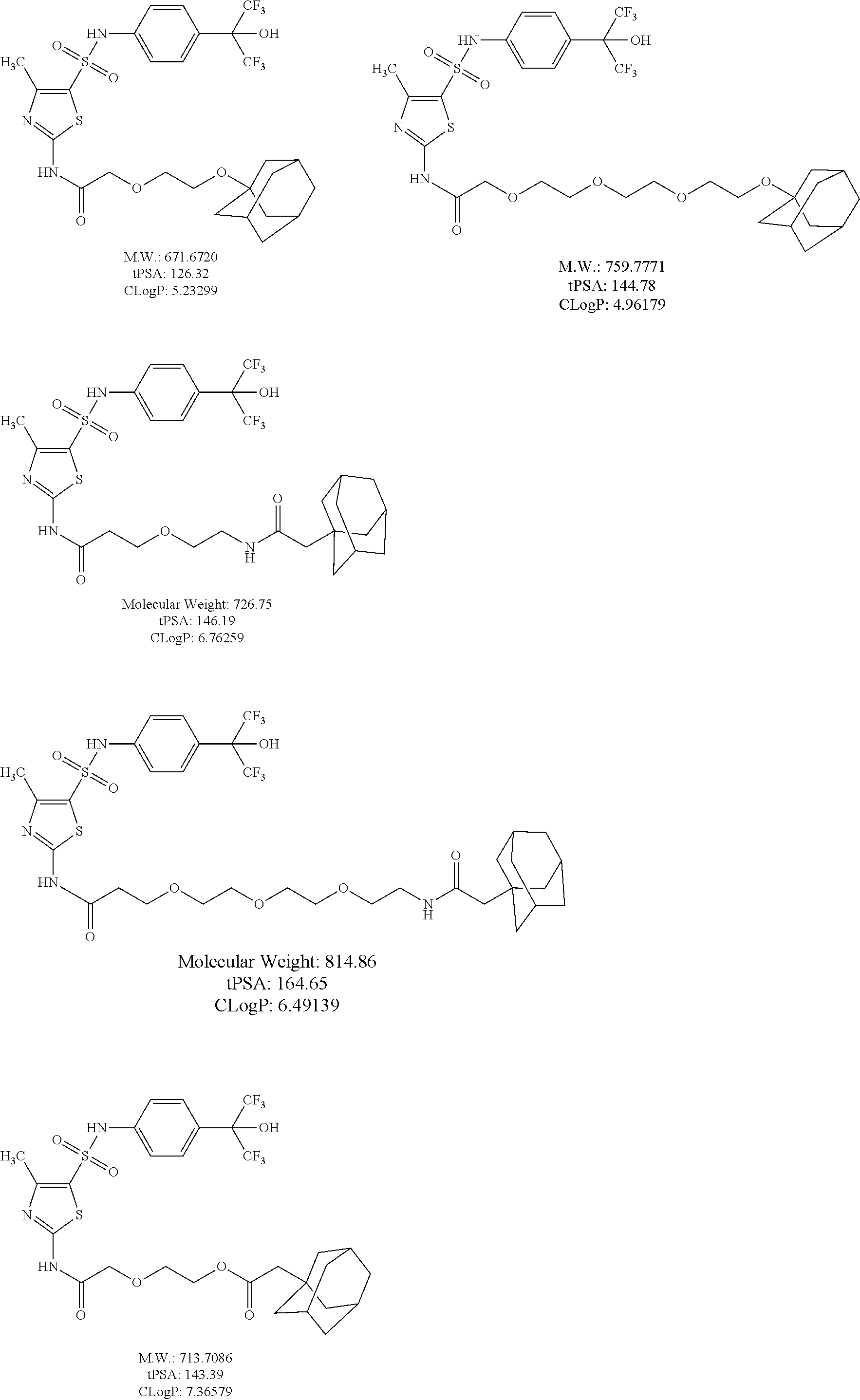 [SMILES: Cc1nc(NC(=O)COCCOC23CC4CC(CC(C4)C2)C3)sc1S(=O)(=O)Nc1ccc(C(O)(C(F)(F)F)C(F)(F)F)cc1 ; Cc1nc(NC(=O)COCCOCCOCCOC23CC4CC(CC(C4)C2)C3)sc1S(=O)(=O)Nc1ccc(C(O)(C(F)(F)F)C(F)(F)F)cc1 ; Cc1nc(NC(=O)CCOCCNC(=O)CC23CC4CC(CC(C4)C2)C3)sc1S(=O)(=O)Nc1ccc(C(O)(C(F)(F)F)C(F)(F)F)cc1 ; Cc1nc(NC(=O)CCOCCOCCOCCNC(=O)CC23CC4CC(CC(C4)C2)C3)sc1S(=O)(=O)Nc1ccc(C(O)(C(F)(F)F)C(F)(F)F)cc1 ; Cc1nc(NC(=O)COCCOC(=O)CC23CC4CC(CC(C4)C2)C3)sc1S(=O)(=O)Nc1ccc(C(O)(C(F)(F)F)C(F)(F)F)cc1]

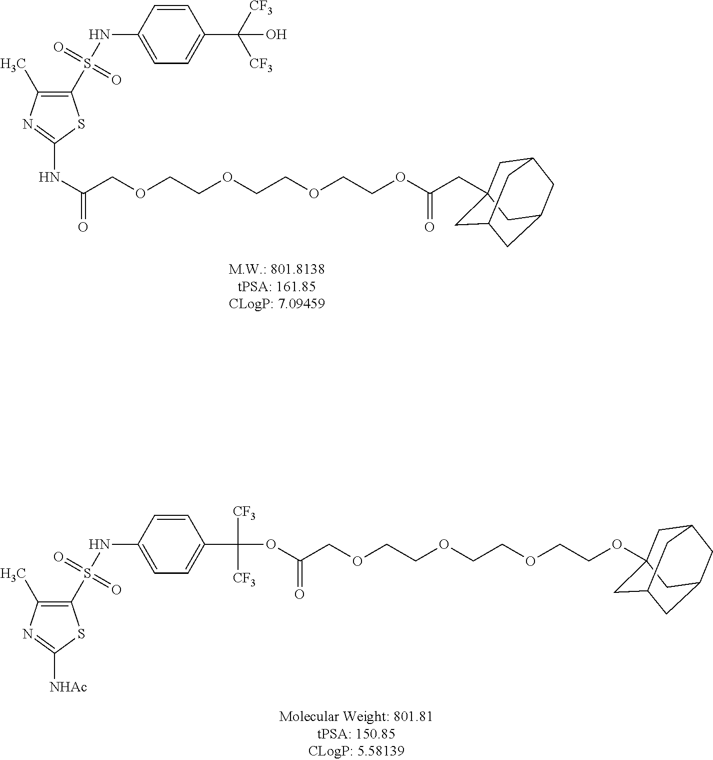 [SMILES: Cc1nc(NC(=O)COCCOCCOCCOC(=O)CC23CC4CC(CC(C4)C2)C3)sc1S(=O)(=O)Nc1ccc(C(O)(C(F)(F)F)C(F)(F)F)cc1 ; CC(=O)Nc1nc(C)c(S(=O)(=O)Nc2ccc(C(OC(=O)COCCOCCOCCOC34CC5CC(CC(C5)C3)C4)(C(F)(F)F)C(F)(F)F)cc2)s1]

Non-limiting examples of compounds that target the estrogen-related receptor α (ERRα) include the following compounds:

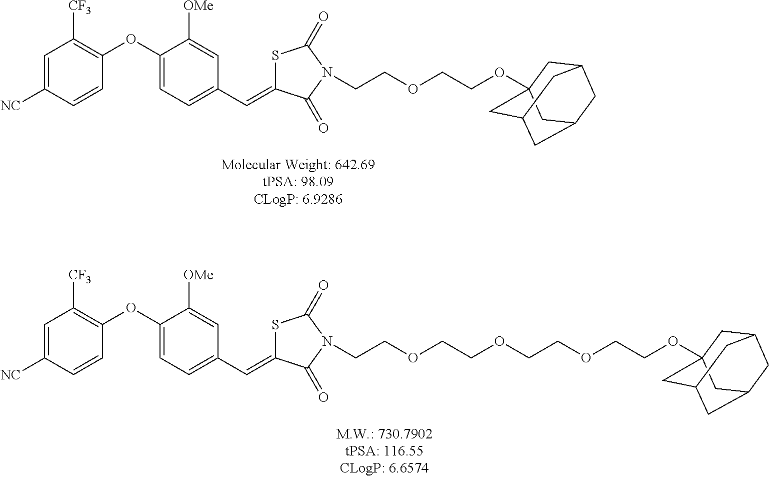 [SMILES: COc1cc(/C=C2\SC(=O)N(CCOCCOC34CC5CC(CC(C5)C3)C4)C2=O)ccc1Oc1ccc(C#N)cc1C(F)(F)F ; COc1cc(/C=C2\SC(=O)N(CCOCCOCCOCCOC34CC5CC(CC(C5)C3)C4)C2=O)ccc1Oc1ccc(C#N)cc1C(F)(F)F]

In one embodiment, the compounds of the invention targets endogenous neurofibrilar tangles (NFT). Without wishing to be limited by theory, using hydrophobic tagging to degrade NFT does not produce potentially neurotoxic intermediary Tau multimers, unlike the situation observed when NFT disassembly is induced with Tau fibrillization inhibitors. In one embodiment, the PBM is a high affinity, BBB-permeable amyloid ligands that are used clinically for PET imaging in patients suffering from AD and other tauopathies. Non-limiting examples of such PBMs are 2-dialkylamino-6-acylmalononitrile (DDNP) analogs that bind the cross-β-sheet motif characteristic of amyloid protein aggregates with high affinity (K_{d}˜0.1 nM), but do not promote disassembly of these species.

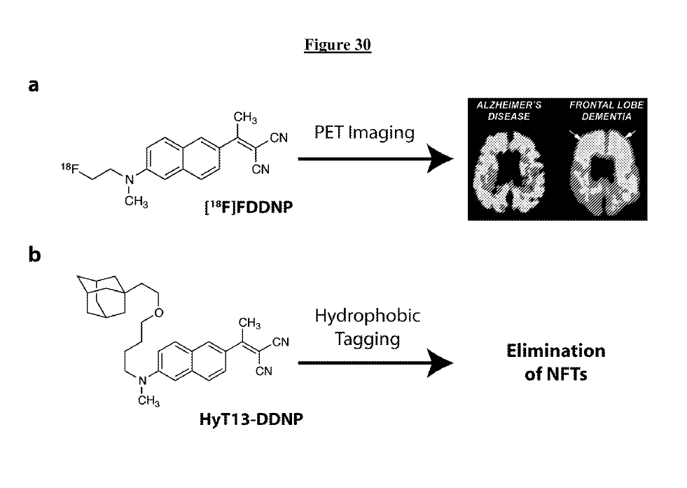

Unlike Congo Red and ThS dyes used to visualize Aβ plaques and NFTs, respectively, in postmortem brain, DDNP derivatives are neutral allowing for uptake across the BBB. In particular, the ¹⁸F-labeled derivative 2-(1-[6-1[(2-¹⁸F]fluoroethyl)(methyl)amino]-2-napthyl}-ethylidene)malononitrile ([¹⁸F]FDDNP) is used clinically for positron emission tomography (PET) imaging of both Aβ plaques and NFTs (FIG. 30). The combination of BBB permeability and an established safety profile make the DDNP pharmacophore an ideal targeting ligand for NFT-directed hydrophobic tags.

In one embodiment, the degron-linker moiety is incorporated in place of ¹⁸F because compounds with ^{99m}Tc chelating groups, which are of similar size to the adamantyl group, at this position retain high affinity binding to amyloid proteins and BBB permeability. In one embodiment, a non-limiting example of such compounds, termed HyT13-DDNP (FIG. 30), maintains the brain uptake of the parent compound based on its low molecular mass (Mᵣ=481 Da) and overall hydrophobicity (C_{logP}=8.19). HyT13-DDNP is evaluated for binding to Tau aggregates in vitro and the ability to resolve NFTs resulting from Tau_{P301L} expression in transgenic mice. For these studies, FDDNP is used to control for changes in NFT abundance that result from DDNP binding, but are independent of hydrophobic tagging.

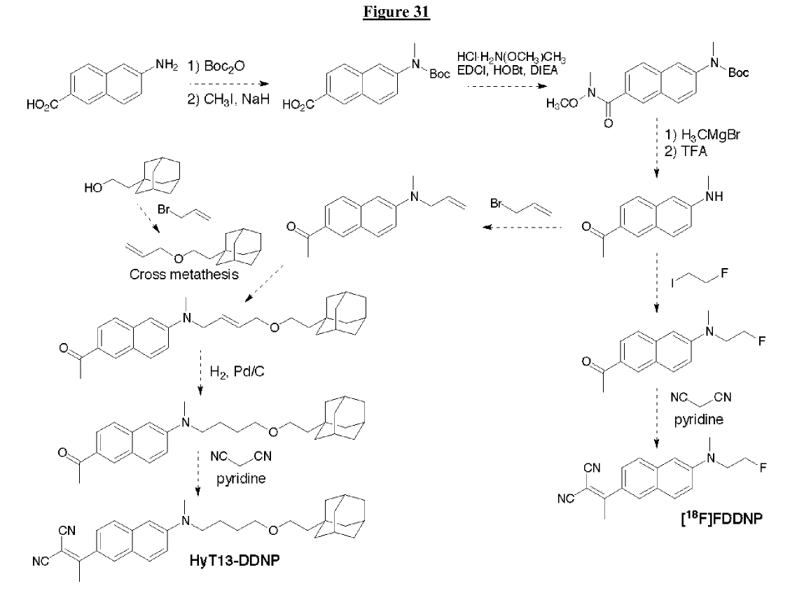

The compounds of the invention may be prepared, for example, according to the chemical sequence illustrated in FIG. 31. Synthesis begins with Boc protection and methylation of commercially available 6-amino-2-napthoic acid (Sigma-Aldrich). The acid is then converted to a ketone via addition of CH₃MgBr to the corresponding Weinreb amide, followed by removal of the Boc group under acidic conditions. Nucleophillic addition of the deprotected secondary amine to 1,2-iodofluoroethane, followed by installation of the malononitrile moiety by condensation of the ketone with dicyanomethane under basic conditions, yields FDDNP.

Synthesis of HyT13-DDNP begins with nucleophillic addition of the deprotected secondary amine to allyl bromide. The adamantyl hydrophobic tag is prepared by nucleophillic addition of the commercially available adamantyl alcohol to allyl bromide. The hydrophobic tag is linked to the DDNP core by olefin cross metathesis followed by reduction of the resulting alkene by hydrogenation. Finally, the malononitrile moiety is installed by condensation of the ketone with dicyanomethane under basic conditions.

### Chemical Synthesis

A generic scheme for the synthesis of compounds of the present invention is described here. Briefly, the compounds may be synthesized pursuant to a general scheme wherein a protein degradation moiety or degron is condensed onto one end of a linker group to provide a linker-degron compound. The linker-degron compound of the present invention has a ClogP of at about 1.5, preferably at least about 2.0, about 3.0 or higher as otherwise described herein.

The invention also contemplates alternative activation procedures. For example, the linker and degron may be activated for further condensation with a protein binding moiety before forming the linker-degron intermediate, or the linker-degron intermediate may be activated for further condensation with a protein binding moiety to provide bifunctional compounds of the present invention.

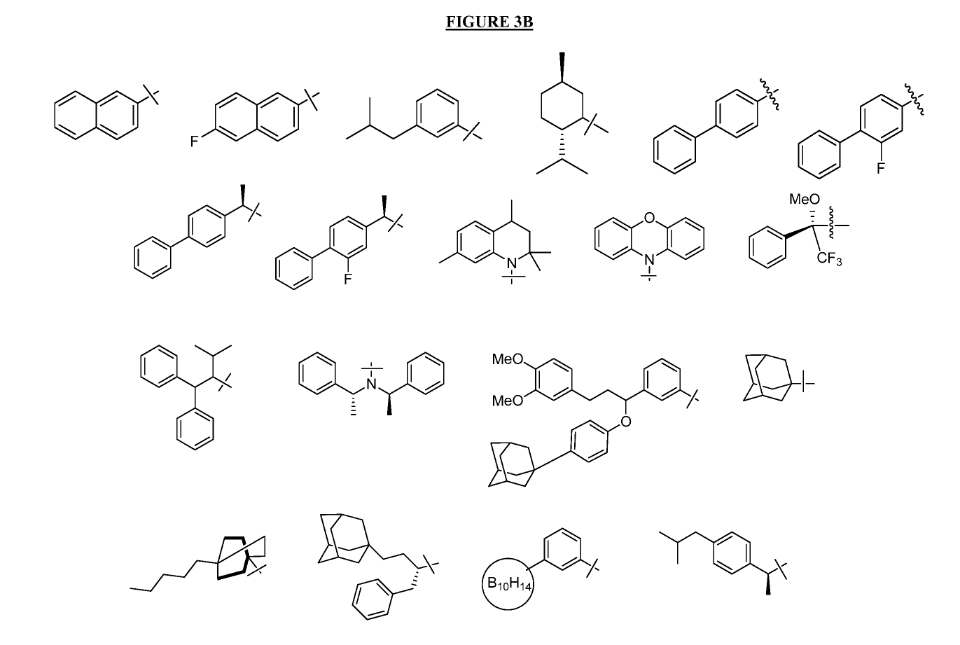

One non-limiting general approach utilized to synthesize compounds of the present invention comprises condensing an ethylene glycol linker pre-assembled with a degron with a protein binding moiety (FIG. 4). The approach may vary, depending upon the degradation moiety and linker used, as well as the chemistry of the protein binding moiety, but the general chemical synthetic scheme is generally applicable to any compound of the present invention.

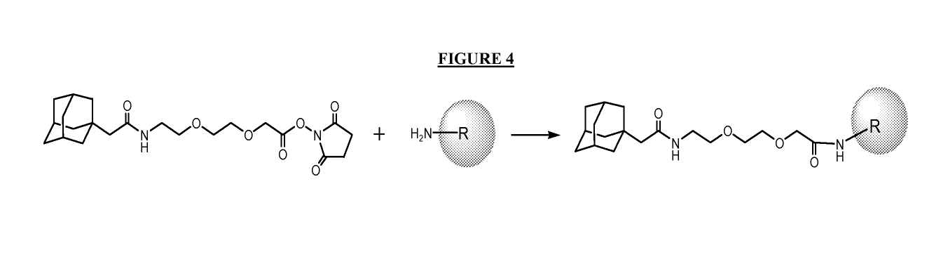

A non-limiting exemplary approach to the chemical synthesis of ethylene glycol-degron intermediates is illustrated in FIG. 5. PEG-building blocks may be linked to degradation moieties comprising distinct adamantyl substituted groups (FIG. 5). Based upon a synthesis by Menger et al., 2006, J. Am. Chem. Soc. 128:1414, commercially available ethylene glycol homologs may be converted to the amines L1-L5 using a three-step-sequence. After monoactivation of the adduct, an azide group may be introduced, and then converted to an amine by means of a Staudinger reaction as described by Menger et al., 2006, J. Am. Chem. Soc. 128:1414. Subsequently, the ethylene glycol homologs 1-5 and the ethylene glycol amines L1-L5 may be coupled to diverse adamantyl derivatives (FIG. 5) to yield homologues of 7, 9 and 11 (x≦5).

### Synthesis of Dimedone-Coupling Reagents:

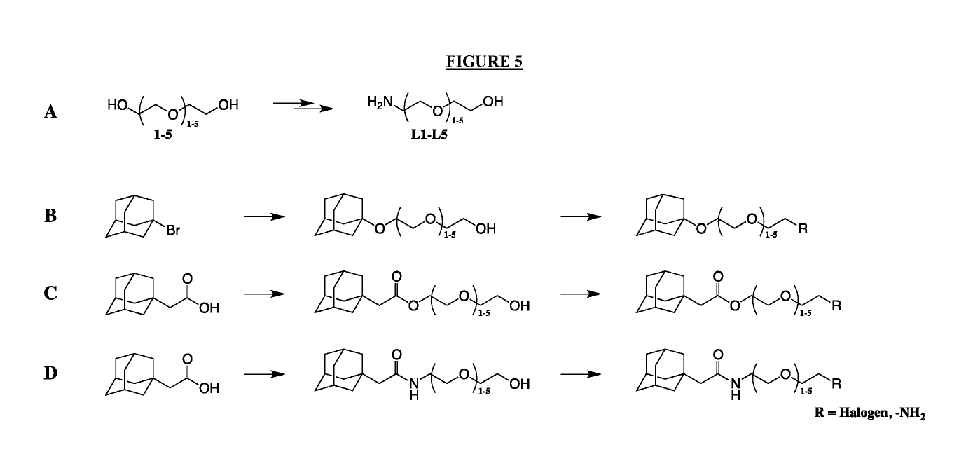

For compounds comprising a dimedone moiety, the dimedone group may be prepared according to the chemistry illustrated in FIGS. 6A-6C. A possible starting material for the synthesis of a 3,5-dioxocyclohexanecarboxylic ester (FIG. 6A) is commercially available 3,5-dihydroxybenzoic acid (12), which is converted to 3,5-diketohexahydrobenzoic acid (13) with the use of Raney nickel and NaOH (van Tamelen and Hildahl, 1956, J. Org. Chem. 4405). Subsequently, compound 13 is protected to afford compound 14, which is coupled to adamantyl-polyethylene glycol-linkers (6, 8, 10, R=OH, x≦5) to afford compounds 15a-c. The final compounds (16a-c) are isolated after deprotection with hydrochloric acid with the use of ceric(IV) ammonium nitrate and a final flash column chromatography.

A possible starting material for the synthesis of C4-substituted cyclohexane-1,3-dione (FIG. 6B) is 1,3-cyclohexanedione (17) (Leonard et al., 2009, ACS Chem. Biol. 128:1414), which is converted to 3-ethoxycyclohex-2-enone (18) using iodine in ethanol. A modification of the C4 position of compound 18 using iodinated adamantyl-polyethylenglycol-linkers (7, 9, 11, R=I, x≦5) follows after an activation for alkylation with lithium diisopropylamide (LDA). The C6-substituted 3-ethoxycyclohex-2-enones (19a-c) are deprotected as described elsewhere herein to yields the final compounds 20a-c.

C5-modified 1,3-cyclohexanedione derivatives may be synthesized from 1,3,5-benzenetriol (21, FIG. 6C). After a selective protection of only one hydroxyl group (22) the compound is reduced to afford the protected 1,3-cyclohexane-dione (23), which is then manipulated to afford compounds 26a-26c.

### Synthesis of Dimedone-Containing Compounds Linked to a Degron:

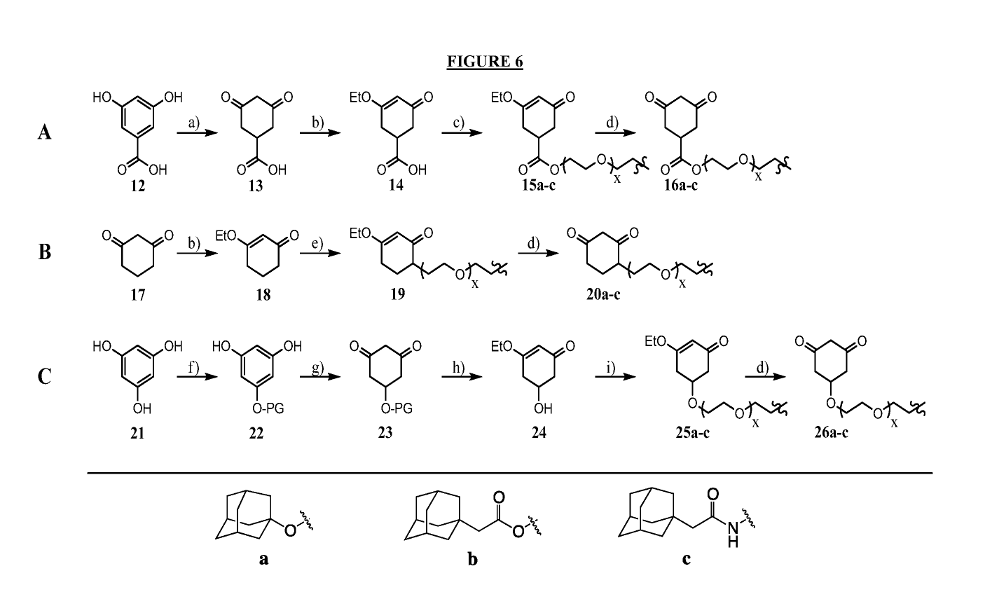

A non-limiting synthetic route to generate compounds of the invention is illustrated in FIGS. 7-10. Commercially available 1-adamantanecarboxylic acid was used as starting material for the synthesis of the amide-amide hydrophobic dimedone compounds. This compound was coupled with N-Boc-ethylenediamine to yield AGR005 (FIG. 7). Acid deprotection of the Boc-protecting group led to the free terminal amine (AGR011) in quantitative yield. This compound was then coupled through an amide bond with the carboxylic acid of the 3,5-dioxocyclohexane-1-carboxylic acid to generate the final product AGR016 as indicated.

Additional compounds with longer PEG linkers were prepared in the subclass of amide-amide hydrophobic dimedone derivatives, using N-Boc-4,7,10-xrioxa-1,13-tridecanediamine for amide formation (AGR014, quantitative yield). After deprotection of the Boc group (AGR020, quant. yield) the amino group was coupled through an amide group to the carboxylic acid of 3,5-dioxocyclohexane-1-carboxylic acid in a final synthetic step.

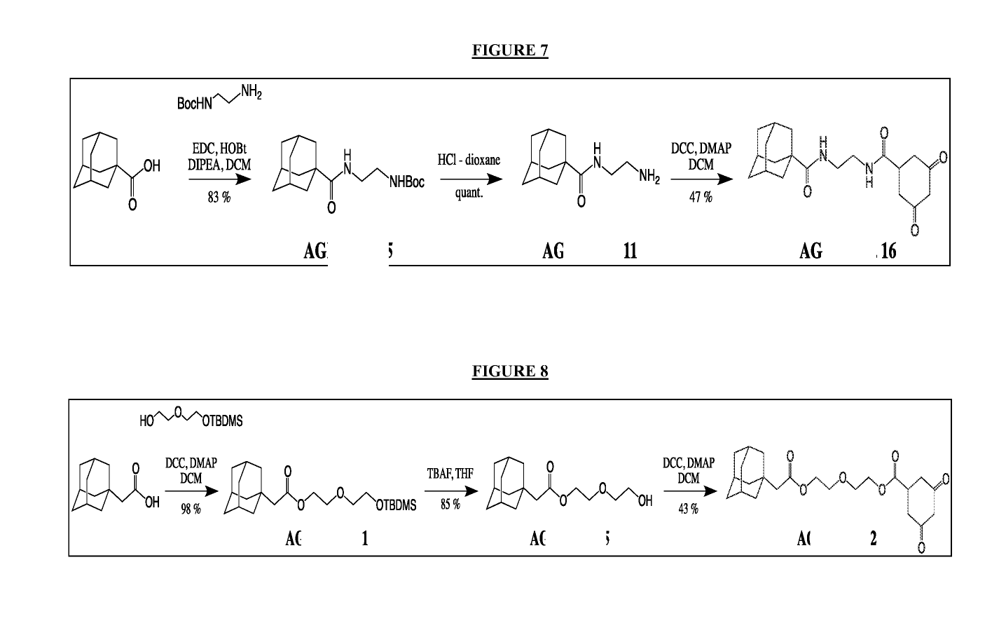

The starting materials for the synthesis of compounds in the subclass of ester-ester compounds were 1-adamantanecarboxylic acid or 1-adamantaneacetic acid, respectively (FIG. 8).

To minimize side products for the first ester formation between either 1-adamantaneacetic acid (FIG. 8) or 1-adamantanecarboxylic acid and a PEG linker, one terminal hydroxyl group of the PEG linker was protected. A suitable protecting group was TBDMS (tert-butyldimethylsilyl), which is unstable under acidic conditions and in the presence of fluoride ion. The mono-TBDMS protection of diethyleneglycol (AGR033, yield: 54%) or pentaethylene glycol (AGR032, yield: 46%) had moderate yield due to the formation of the diprotected product. Ester formation between the adamantyl carboxylic acid derivative and the monoprotected PEG linker had yields between 44 and 98%. TBDMS deprotection in the presence of TBAF (tert-butyl ammonium fluoride) occurred with good yields (85-92%), and the resulting alcohol was coupled through an ester linkage to 3,5-dioxocyclohexane-1-carboxylic acid to form the final products AGR042 (43%), AGR043 (56%), AGR047 (29%) and AGR049 (64%).

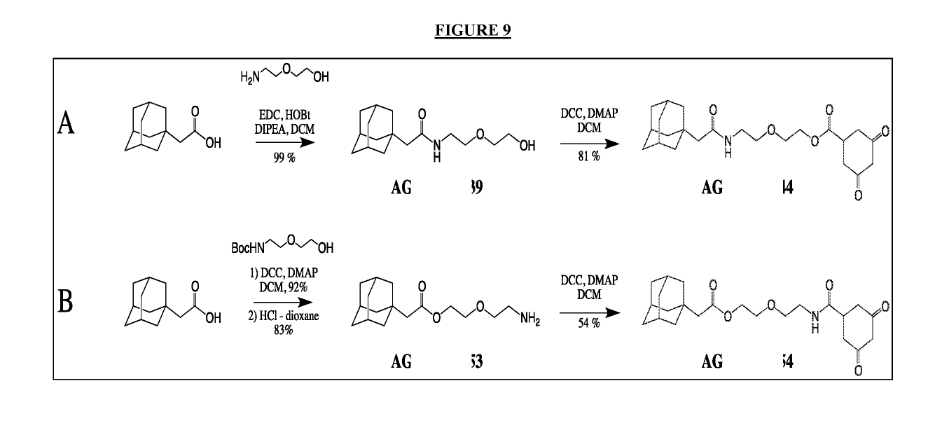

To synthesize compounds comprising both an amide and an ester bond, two distinct strategies were used. In case where the amide bond was formed with the adamantyl-containing group, commercially available compounds were used without a previous protection/deprotection of the 2-(2-aminoethoxy) ethanol (FIG. 9A), since the amino group was more nucleophilic than the hydroxyl group. In case where the amide bond was formed with the 3,5-dioxocyclohexane-1-carboxylic acid (and the adamantyl group is connected through an ester bond), the linker 2-(2-aminoethoxy) ethanol was protected at the amino function (FIG. 9B). All other steps were equivalent to the strategies described elsewhere herein.

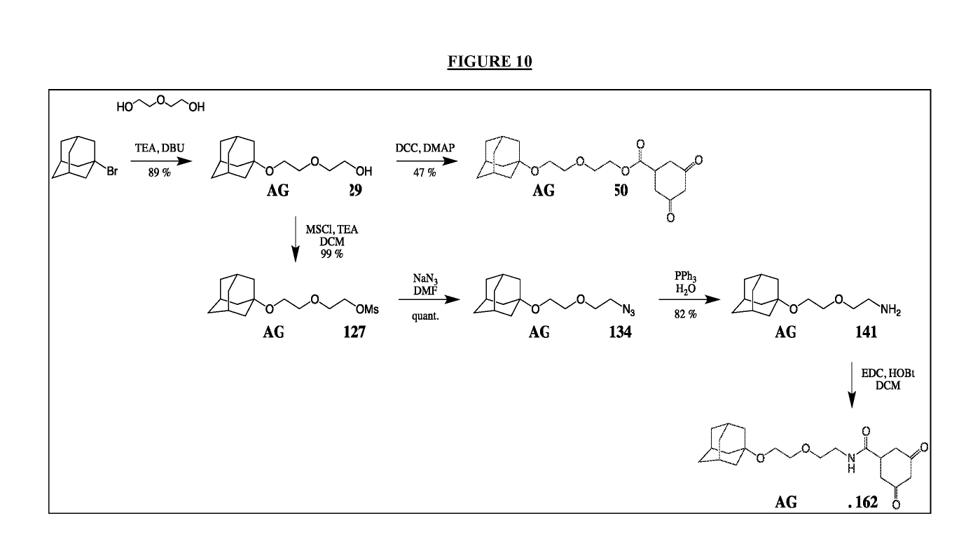

For the synthesis of hydrophobic dimedone compounds wherein the adamantyl group was connected through an ether bond, the starting materials were commercially available compounds (FIG. 10). This synthetic route involved ether formation of 1-bromoadamantane in the presence of diethylene glycol or higher homologues. General procedures, such as TEA (triethyl amine) with heating to 110° C. or NaH treatment in DMF, were not successful. Only in the presence of a catalytically amount of DBU (a non-nucleophilic base) with equimolar amounts of TEA could the desired product be isolated after heating to 110° C. for 15 h. Using this prescription all four homologues (with linkers diethylene glycol, triethylene glycol, tetraethylene glycol and pentaethylene glycol) were synthesized in good yields (between 58-99%). Ester formation between the resulting alcohols and the dimedone derivative was carried out under standard conditions (DCC, DMAP, DCM, RT, 18 h).

To generate ether-amide derivatives (FIG. 10), AGR029 as well as the higher homologues (AGR057—triethylene glycol; AGR058—tetraethylene glycol and AGR063—pentaethylene glycol) were converted to mesylates, which contain a better leaving group to allow for azide displacement. After Staudinger reduction of the azide, the terminal amine AGR141 was then coupled through an amide bond with 3,5-dioxocyclohexane-1-carboxylic acid. Apoptosis assays showed that two compounds containing ether-amide linkages showed promising biological activity.

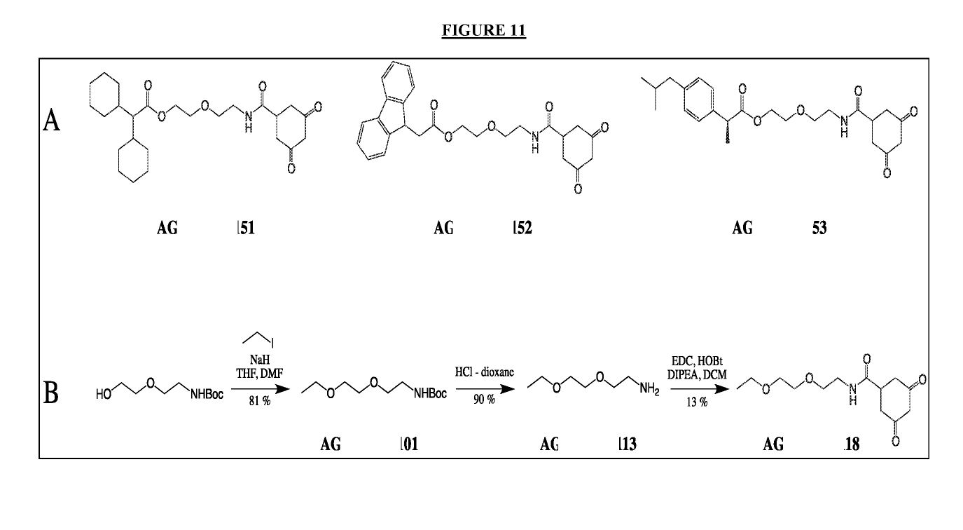

Synthetic efforts were also directed to substituting other protein degradation moieties for the adamantyl group, and the impact of this substitution on biological activity was evaluated. The compounds illustrated in FIG. 11A were synthesized using the same conditions as for compound AGR054 (FIG. 9B). All of these compounds were active in in vitro apoptosis assays, but less active than AGR054 (adamantyl group).

As a control compound, synthesis of AGR118 was carried out (FIG. 11B), wherein the adamantyl ester group was substituted with an ethyl ether. As a first step, the Boc protected 2-(2-aminoethoxy) ethanol was subjected to a Williamson ether synthesis using iodoethane. Subsequently, the Boc group was removed by acid treatment, and the resulting amino group was coupled to 3,5-dioxocyclohexane-1-carboxylic acid. Compound AGR118 showed no apoptotic properties, indicating that the hydrophobic head group is required for a biological activity.

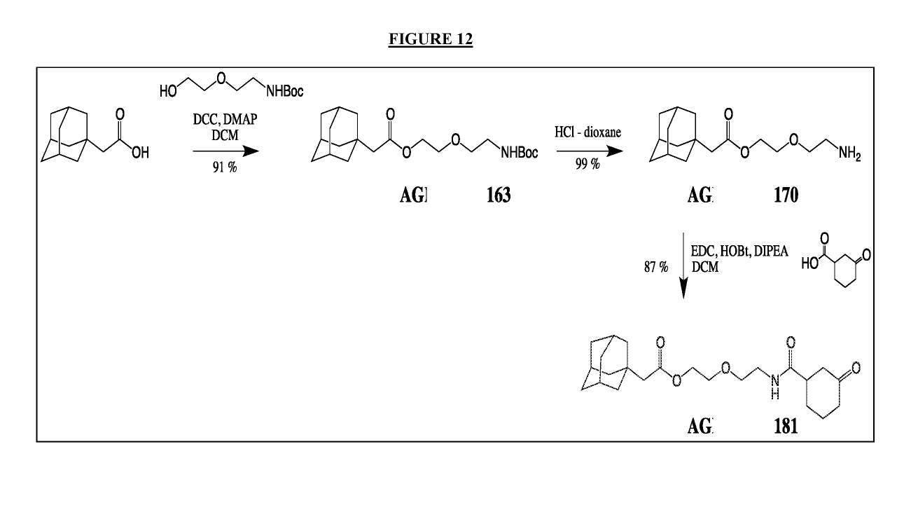

Another control compound AGR181 (FIG. 12) was prepared to assess the importance of the dimedone head group. Compound AGR181 lacks a second keto group compared to the dimedone ring, and thus the 2-position (which is involved in forming an irreversible covalent bond with sulfenic acid) is much less acidid than compound AGR054. Consistently, AGR181 showed no activity in cell culture experiments.

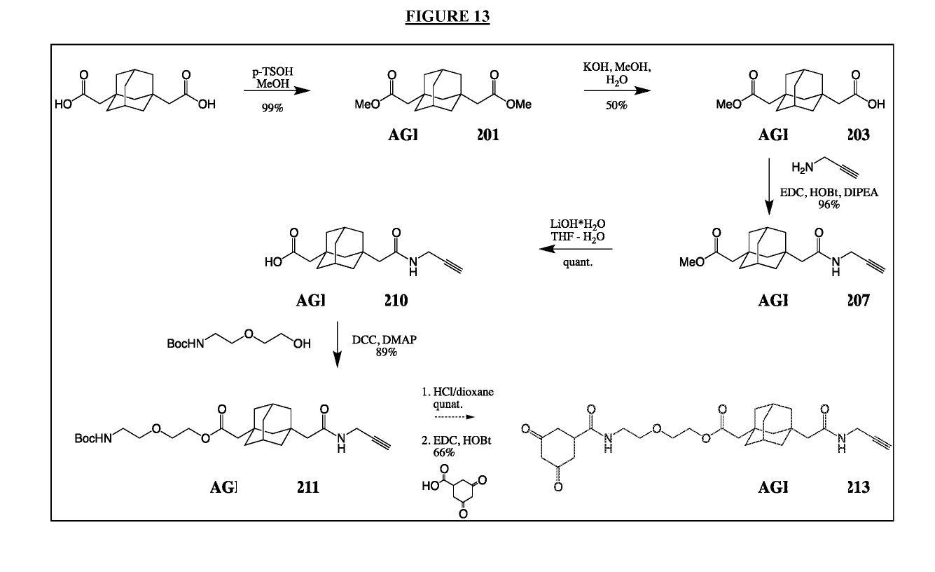

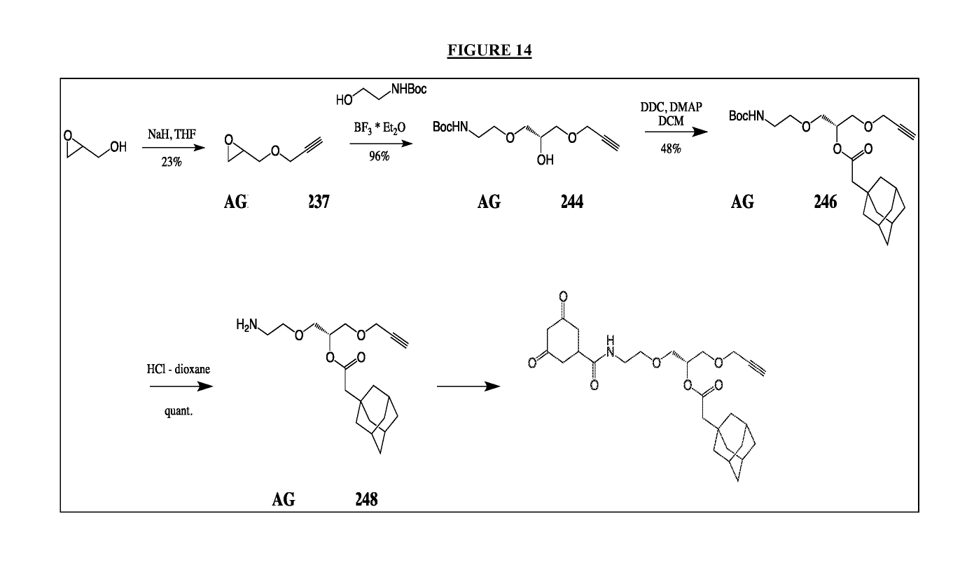

To evaluate which proteins are tagged using compound AGR054, a pull down reagent with similar structure to AGR054 but with an additional alkyne group was prepared (FIG. 13). The alkyne group was directly coupled to the adamantyl head group through an amide bond. Attempts to insert the alkyne group through alkylation of 3-hydroxyadamantaneacetic acid were not successful. Instead, K₂CO₃, DMF, propargyl bromide conditions (AGR186 and AGR187) as well as NaH, THF and propargyl bromide conditions (AGR200) were used. The pull down reagent AGR213 was less active than the original compound, probably because of the modified lipophilicity of the adamantyl head group. This issue may be addressed by preparing pull down reagent AGR248 (FIG. 14). In one aspect, in AGR 248 the alkyne group is connected with the linker itself and the adamantyl head group as well as the dimedone pattern are undisturbed.

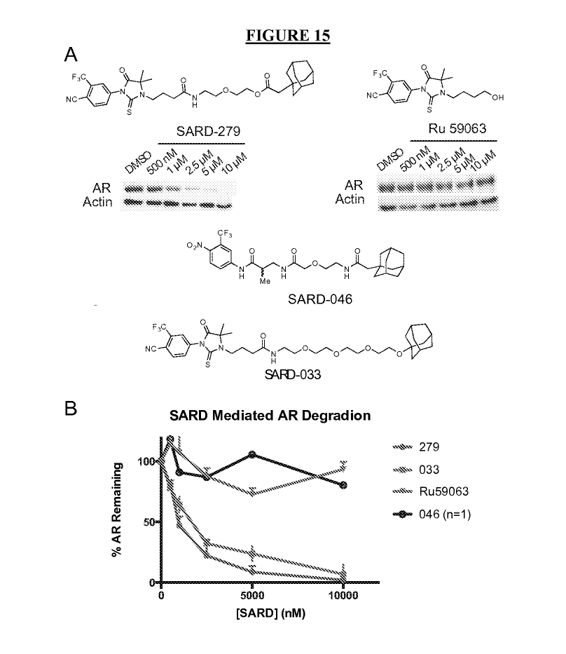

For those compounds that contain androgen receptor binding moieties (SARDS, based upon flutamide, bicalutamide or RU59063; FIG. 15), the protein binding moieties were readily derivatized by condensing a linker group onto a readily prepared degron derivative and then manipulating the resulting molecule to install an electrophilic group that may condense with the protein binding moiety. FIG. 15 illustrates representative compounds prepared with androgen receptor protein binding moieties using the general approach described above.

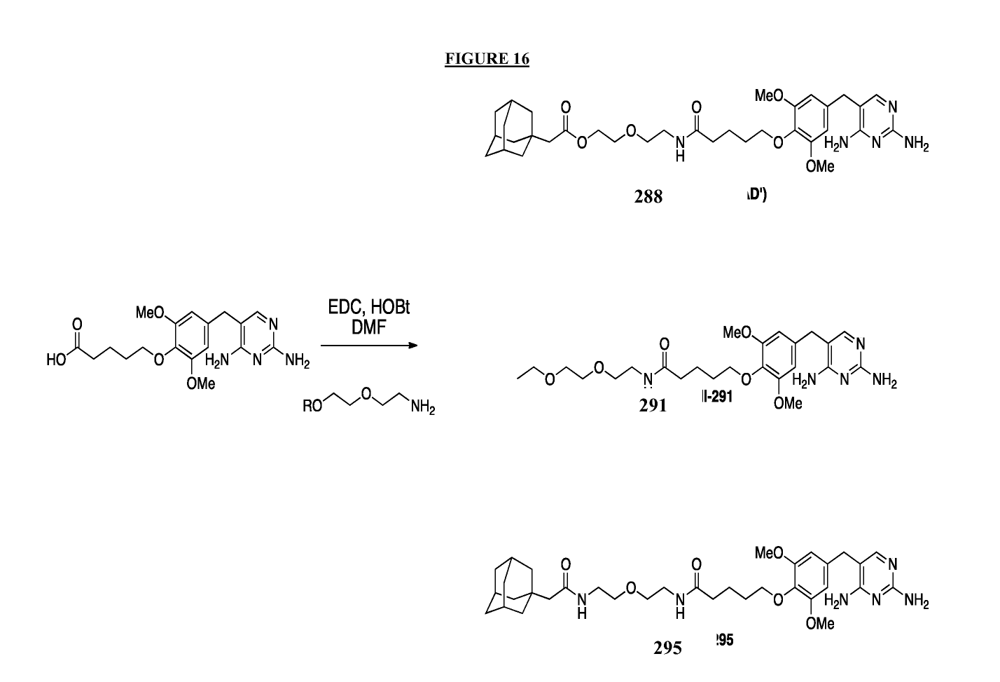

Those compounds comprising a TMP group as the protein binding moieties were readily synthesized from TMP acid (FIG. 16). In short, TMP acid was synthesized (Cornish et al., 2007, ChemBioChem. 8:767-774) and then condensed with a linker group (preferably to which a degron such as an adamantyl group had been previously attached), thus producing the TMP based compounds of the present invention. Representative compounds are illustrated in FIG. 16.

### Salts

The compounds described herein may form salts with acids, and such salts are included in the present invention. In one embodiment, the salts are pharmaceutically acceptable salts. The term “salts” embraces addition salts of free acids or bases that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound.

### Combination Therapies

The compounds of the formula (I) or a pharmaceutically acceptable salt thereof may be used in combination with or include one or more other therapeutic agents and may be administered either sequentially or simultaneously by any convenient route in separate or combined pharmaceutical compositions.

The compounds of formula (I) or a pharmaceutically acceptable salt thereof and further therapeutic agent(s) may be employed in combination by administration simultaneously in a unitary pharmaceutical composition including both compounds. Alternatively, the combination may be administered separately in separate pharmaceutical compositions, each including one of the compounds in a sequential manner wherein, for example, the compound of formula or a pharmaceutically acceptable salt thereof is administered first and the other second and vice versa. Such sequential administration may be close in time (e.g., simultaneously) or remote in time. Furthermore, it does not matter if the compounds are administered in the same dosage form, e.g., one compound may be administered topically and the other compound may be administered orally. Suitably, both compounds are administered orally. During a treatment regime, it will be appreciated that administration of each agent of the combination may be repeated one or more times.

When the combination is administered separately in a sequential manner wherein one is administered first and the other second or vice versa, such sequential administration may be close in time or remote in time. For example, administration of the other agent several minutes to several dozen minutes after the administration of the first agent, and administration of the other agent several hours to several days after the administration of the first agent are included, wherein the lapse of time is not limited, For example, one agent may be administered once a day, and the other agent may be administered 2 or 3 times a day, or one agent may be administered once a week, and the other agent may be administered once a day and the like.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art.

When the compound of compound of formula (I) or a pharmaceutically acceptable salt thereof is used in combination with further therapeutic agent or agents active against the same disease, condition or disorder the dose of each agent may differ from that when the compound is used alone. Appropriate doses will be readily appreciated by those skilled in the art.

In one aspect, the compounds of the invention are useful in the methods of present invention in combination with at least one additional compound useful for preventing and/or treating cancer or an oxidative distress disease state. These additional compounds may comprise compounds of the present invention or other compounds, such as commercially available compounds, known to treat, prevent, or reduce the symptoms of cancer or an oxidative distress disease state. In one embodiment, the combination of at least one compound of the invention or a salt thereof and at least one additional compound useful for preventing and/or treating cancer or an oxidative distress disease state has additive, complementary or synergistic effects in the prevention and/or treatment of cancer or an oxidative distress disease state.

In one embodiment, a compound of the invention is used in combination with an additional bioactive agent, such as a HSP 90 or HSP 70 antagonist and/or agonist.

In one aspect, the present invention contemplates that a compound useful within the invention may be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In one embodiment, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In another embodiment, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

As used herein, combination of two or more compounds may refer to a composition wherein the individual compounds are physically mixed or wherein the individual compounds are physically separated. A combination therapy encompasses administering the components separately to produce the desired additive, complementary or synergistic effects. In one embodiment, the compound and the agent are physically mixed in the composition. In another embodiment, the compound and the agent are physically separated in the composition.

In one embodiment, the compound of the invention is co-administered with a compound that is used to treat cancer or an oxidative distress disease state. The co-administered compound may be administered individually, or a combined composition as a mixture of solids and/or liquids in a solid, gel or liquid formulation or as a solution, according to methods known to those familiar with the art.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Methods

The invention includes a method of degrading a protein in a cell or tissue of a subject in need thereof. The method comprises administering to the subject a therapeutically effective amount of a pharmaceutical composition comprising a compound of the invention, whereby the protein is degraded in the cell or tissue of the subject. In one embodiment, the subject is further administered an additional bioactive agent. In another embodiment, the protein degradation results in cell apoptosis. In yet another embodiment, the protein comprises a sulfenome protein, Bcr-Abl tyrosine kinase, or dihydrofolate reductase.

The invention also includes a method of treating or preventing a disease or disorder that is associated with a protein in a subject. The method comprises administering to the subject a therapeutically effective amount of a pharmaceutical composition comprising a compound of the invention. In one embodiment, the subject is further administered an additional bioactive agent.

The invention also includes a method of treating or preventing in a subject in need thereof an oxidative stress disease state or a disease wherein a sulfenome protein is present in the diseased cells of the subject. The method comprises administering to the subject a therapeutically effective amount of a pharmaceutical composition comprising a compound of the invention. In one embodiment, the subject is further administered an additional bioactive agent. In another embodiment, the disease state or condition is cancer, hyperproliferative cell growth conditions, Parkinson's disease, Alzheimer's disease, atherosclerosis, heart failure (including congestive heart failure), myocardial infarction, schizophrenia, bipolar disorder, fragile X syndrome, sick cell disease, chronic fatigue syndrome, aging (including aging by induction of mitohormesis, diabetes (including type I) and vascular disease.

The invention also includes a method of controlling protein levels within a cell of a subject. The method comprises treating the subject with a therapeutically effective amount of a pharmaceutical composition comprising a compound of the invention, whereby the protein levels in the cell of the subject are controlled. In one embodiment, the compounds of the invention interact with a specific target protein such that degradation of the target protein in vivo result in the control of the protein amounts in the biological system, preferably for a particular therapeutic benefit. As described elsewhere herein, representative compounds of the present invention exhibited substantial activity in inducing protein degradation.

### Kits

The invention includes a kit comprising an applicator, an instructional material for use thereof, and a compound of the invention. In one embodiment, the instructional material included in the kit comprises instructions for degrading a protein in a cell or tissue of a subject. In another embodiment, the instructional material included in the kit comprises instructions for treating a disease or disorder that is associated with a protein. In yet another embodiment, the instructional material included in the kit comprises instructions for treating an oxidative stress disease state or a disease wherein a sulfenome protein is present in the diseased cells of a subject.

The instructional material recites that the subject is administered a therapeutically effective amount of a pharmaceutical composition comprising the compound contained in the kit. In one embodiment, the disease or disorder comprises cancer or an oxidative stress disease state. In another embodiment, the cancer comprises breast cancer, prostate cancer, melanoma, and any combinations thereof.

The combinations of the invention may also be presented as a combination kit. When the agents of the combination are administered simultaneously, the combination kit can contain the agents in a single pharmaceutical composition, such as a tablet, or in separate pharmaceutical compositions. When the agents are not administered simultaneously, the combination kit will contain each agent in separate pharmaceutical compositions either in a single package or in separate pharmaceutical compositions in separate packages.

The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

### Pharmaceutical Compositions and Formulations

The invention includes the use of at least one compound of the invention or a salt thereof to practice the methods of the invention. In one embodiment, the compound is part of a pharmaceutical composition.

Such a pharmaceutical composition may consist of at least one composition of the invention or a salt thereof, in a form suitable for administration to a subject, or the pharmaceutical composition may comprise at least one composition of the invention or a salt thereof, and one or more pharmaceutically acceptable carriers, one or more additional ingredients, or some combination of these. The composition of the invention may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, liposomal preparations, coated particles, resealed erythrocytes containing the active ingredient, and immunologically-based formulations. The route(s) of administration are readily apparent to the skilled artisan and depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of at least one composition of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material (such as a medical device or stent) with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of the disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound is from about 0.01 mg/kg to 100 mg/kg of body weight/per day. One of ordinary skill in the art is able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compositions of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of the disease or disorder in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compositions of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments thereinbetween.

In some embodiments, the dose of a composition of the invention is from about 0.5 μg and about 5,000 mg. In some embodiments, a dose of a composition of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a composition of the invention, alone or in combination with a second pharmaceutical agent; and instructions for using the compound to treat, prevent, or reduce one or more symptoms of the disease or disorder in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing the disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Injectable formulations may also be prepared, packaged, or sold in devices such as patient-contolled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Application Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment, the compositions of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 24 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials

Unless otherwise noted, materials were obtained from commercial suppliers and used without purification.

### Example 1

### 4-(3-(4-Cyano-3-(trifluoromethyl)phenyl)-5,5-dimethyl-4-oxo-2-thioxoimidazolidin-1-yl)butanoic acid (Ru-Acid)

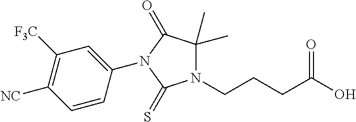 [SMILES: CC1(C)C(=O)N(c2ccc(C#N)c(C(F)(F)F)c2)C(=S)N1CCCC(=O)O]

RU59063 (4-[3-(4-hydroxybutyl)-4,4-dimethyl-5-oxo-2-thioxoimidazolidin-1-yl]-2-(trifluoromethyl)benzonitrile; 145 mg, 0.38 mmol) was dissolved in 2 mL DMF and charged with PDC (1.4 g, 3.7 mmol) and stirred for 48 hours, and then the mixture was quenched with 10 mL 1 M HCl and extracted into Et₂O (5×25 mL). The combined organic layers were washed with brine (1×100 mL), dried with Na₂SO₄ and concentrated down to yield 135 mg (90% yield) pure product. ¹H NMR (300 MHz, CDCl₃) δ 7.94 (d, J=8.3, 1H), 7.88 (s, 1H), 7.77 (d, J=8.2, 1H), 3.82-3.65 (m, 2H), 2.50 (s, 2H), 2.14 (s, 2H), 1.59 (s, 6H); ¹³C NMR (126 MHz, CDCl₃) δ 178.6, 177.4, 175.3, 175.2, 137.1, 135.2, 133.5 (q, J=32.1), 132.1, 127.0 (q, J=4.7), 121.8 (q, J=276.2), 114.9, 110.1, 65.2, 43.3, 31.7, 23.1; LRMS (ESI) 421.2 (M+Na)⁺.

### Example 2

### 2-(Adamantan-1-yl)-N-(2-(2-aminoethoxyl)ethyl) acetamide

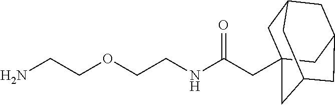 [SMILES: NCCOCCNC(=O)CC12CC3CC(CC(C3)C1)C2]

To a round bottom flask with stirbar was charged 1-adamantaneacetic acid (1.0 g, 9.7 mmol), EDC (1.43 g, 7.5 mmol), HOBt (1.16 g, 7.5 mmol), and 20 mL dichloromethane. After 15 minutes of stirring diamine (1.1 g, 10.0 mmol) was added and the mixture left stir for 16 h upon which the mixture was diluted with 30 mL dichloromethane and washed with saturated Na₂CO₃ (2×50 mL). The organic layer was dried with Na₂SO₄ and concentrated down to yield a crude oil that was purified by silica gel chromatography (dichloromethane to 4:1 dichloromethane:MeOH (0.5 N NH₃)) to yield 520 mg (35% yield) of pure product as an amber oil. ¹H NMR (300 MHz, CDCl₃) δ 3.50-3.33 (m, 8H), 2.78 (t, J=5.1, 2H), 1.95-1.85 (m, 6H), 1.65-1.45 (m, 9H); ¹³C NMR (75 MHz, CDCl₃) δ 171.1, 77.3, 72.7, 69.7, 51.4, 42.5, 38.9, 36.7, 32.6, 28.6; LRMS (ESI) 281.3 (M+H)⁺.

### Example 3

### 2-(2-(2-(2-(Adamantan-1-yloxy)ethoxy)ethoxy)ethoxy) ethanamine

### 2-(2-(2-(2-(Adamantan-1-yl-oxy)ethoxy)ethoxy)ethoxy)ethanol

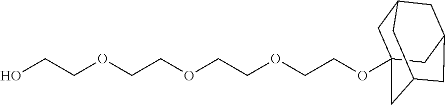 [SMILES: OCCOCCOCCOCCOC12CC3CC(CC(C3)C1)C2]

To a round bottom flask with stirbar was charged 2-(2-(2-(2-hydroxy-ethoxy)ethoxy)ethoxy)ethanol (4.5 g, 23.3 mmol), 1-bromoadamantane (1.0 g, 4.6 mmol), Et₃N (2.1 mL 15.0 mmol) and DBU (0.033 mL, 0.23 mmol). Upon stirring at 110° C. for 18 h the reaction was diluted with 25 mL 1 M aq. HCl and extracted in to dichloromethane (2×25 mL). The organic layer was washed with water (2×25 mL) and dried with Na₂SO₄ to yield a crude oil. Column chromatography (4:1 Hex:EtOAc to 100% EtOAc) led to the isolation of 202 mg (30% yield) pure product. ¹H NMR (500 MHz, CDCl₃) δ 3.69-3.63 (m, 2H), 3.62-3.57 (m, 8H), 3.56-3.46 (m, 6H), 3.30 (s, 1H), 2.07 (s, 3H), 1.76-1.62 (m, 6H), 1.54 (q, J=12.2, 6H); ¹³C NMR (126 MHz, CDCl₃) δ 73.0, 72.6, 71.5, 70.9, 70.9, 70.9, 70.6, 61.9, 59.6, 41.7, 36.8, 30.8; LRMS (ESI) 329.5 (M+H)⁺.

### 2-(2-(2-(2-(Adamantan-1-yloxy)ethoxy)ethoxy)ethoxy)ethyl methanesulfonate

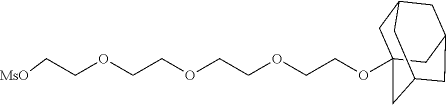 [SMILES: CS(=O)(=O)OCCOCCOCCOCCOC12CC3CC(CC(C3)C1)C2]

To a round bottom flask with stirbar and 5 mL distilled dichloromethane was charged 2-(2-(2-(2-(adamantan-1-yl-oxy)ethoxy)ethoxy)ethoxy)ethanol (200 mg, 0.6 mmol), methanesulfonyl chloride (70 μL, 0.9 mmol) and Et₃N (253.0 μL, 1.8 mmol). Upon stirring at room temperature for 18 h the reaction was diluted with 5 mL 1 M aq. HCl and extracted in to dichloromethane (2×5 mL). The organic layer was washed with water (2×10 mL) and dried with Na₂SO₄ to yield a crude oil. Column chromatography (3:1 Hex:EtOAc to 100% EtOAc) led to the isolation of 200 mg (82% yield) pure product. ¹H NMR (300 MHz, CDCl₃) δ 4.43-4.32 (m, 2H), 3.82-3.71 (m, 2H), 3.70-3.61 (m, 8H), 3.57 (s, 2H), 3.08-3.06 (m, 2H), 2.13 (s, 3H), 1.73 (m, 6H), 1.68-1.51 (m, 6H) ¹³C NMR (75 MHz, CDCl₃) δ 72.3, 71.3, 70.6, 70.6, 70.5, 70.5, 69.3, 69.0, 59.2, 41.4, 39.4, 37.3, 30.5; LRMS (ESI) 405.8 (M+H)⁺.

### 1-(2-(2-(2-(2-Azidoethoxy)ethoxy)ethoxy)ethoxy)adamantane

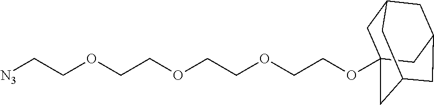 [SMILES: [N-]=[N+]=NCCOCCOCCOCCOC12CC3CC(CC(C3)C1)C2]

To a 2 dram vial with stirbar and 3 mL DMF was charged 2-(2-(2-(2-((adamantan-1-yloxy)ethoxy)ethoxy)ethoxy)ethyl methanesulfonate (300 mg, 0.74 mmol) and sodium azide (120 mg, 1.85 mmol). Upon stirring at 80° C. for 18 h the reaction was diluted with 5 mL H₂O and extracted into EtOAc (2×5 mL). The organic layer was washed with water (2×10 mL) and dried with Na₂SO₄, and concentrated down to yield a crude oil. Column chromatography (dichloromethane to 10:1 dichloromethane:MeOH) resulted in the recovery of 172 mg (63% yield) of pure product. ¹H NMR (500 MHz, CDCl₃) δ 3.66-3.58 (m, 11H), 3.54 (s, 3H), 3.34 (s, 2H), 2.09 (s, 3H), 1.69 (s, 6H), 1.56 (q, J=12.0, 6H). ¹³C NMR (125 MHz, CDCl₃) δ 72.2, 71.2, 70.7, 70.6, 70.6, 70.5, 70.0, 59.2, 50.6, 41.4, 36.4, 30.4; LRMS (ESI) 326.6 (M-N₂).

### 2-(2-(2-(2-(Adamantan-1-yloxy)ethoxy)ethoxy)ethoxy)ethanamine

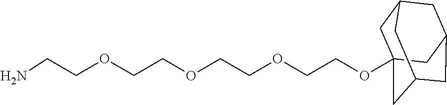 [SMILES: NCCOCCOCCOCCOC12CC3CC(CC(C3)C1)C2]

To a 2 dram vial with stirbar and 2 mL THF was charged 1-(2-(2-(2-(2-azidoethoxyl)ethoxy)ethoxy)ethoxy)adamantane (170 mg, 0.47 mmol) and triphenylphosphine (150 mg, 0.57 mmol). 20 μL of H₂O was added after 2 h and the mixture let stir for 16 h at room temperature. The solvents were removed under vacuum and the mixture diluted with 5 mL 1 M aq. HCl and washed with EtOAc (2×5 mL). The aqueous layer was basified with 20 mL 3 M NaOH and the product was extracted into dichloromethane (4×25 mL). The organic layer was washed with water (2×10 mL) and dried with Na₂SO₄, and concentrated down to yield 85 mg (55% yield) of product. ¹H NMR (500 MHz, CDCl₃) δ 3.70-3.56 (m, 8H), 3.54 (s, 4H), 3.48 (t, J=4.9, 2H), 2.83 (s, 2H), 2.09 (s, 3H), 1.70 (s, 6H), 1.56 (q, J=12.3, 6H); ¹³C NMR (125 MHz, CDCl₃) δ 73.0, 72.3, 71.2, 70.5, 70.5, 70.5, 70.2, 59.2, 41.6, 41.4, 36.4, 30.4; LRMS (ESI) 327.4 (M+H)⁺.

### Example 4

### 2-(2-(2-(Adamantan-1-yl)ethoxy)ethoxy)ethanamine

### 2-(2-(2-(Adamantan-1-yl)ethoxy)ethoxy)ethanol

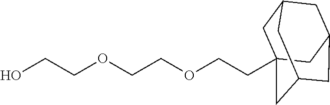 [SMILES: OCCOCCOCCC12CC3CC(CC(C3)C1)C2]

To a round bottom flask with stirbar was charged 60% NaH (400 mg, 10.0 mmol), which was then sparged with argon and suspended in dry DMF (20 mL) and cooled to 0° C. 2,2′-Oxydiethanol (0.5 g, 5.0 mmol) was added and the mixture was stirred for 45 minutes. Then 1-(2-iodoethyl)adamantane (300 mg, 1.05 mmol) was added and the reaction was allowed to warm to room temperature and stirred for 18 h. The reaction was quenched with 25 mL saturated NH₄Cl and extracted into EtOAc (3×25 mL). The organic layer was then washed with brine (3×30 mL) and concentrated down to yield a crude oil which was purified by silica gel chromatography (5:1 to 1:1 hexanes:EtOAc to yield 55 mg (20% yield) of pure product. ¹H NMR (500 MHz, CDCl₃) δ 3.79-3.72 (m, 2H), 3.68 (dd, J=3.7, 5.6, 2H), 3.65-3.62 (m, 2H), 3.59 (dd, J=3.7, 5.6, 2H), 3.53 (dd, J=5.2, 10.1, 2H), 1.94 (s, 3H), 1.70 (d, J=12.1, 3H), 1.63 (d, J=10.8, 6H), 1.52 (d, J=2.4, 6H), 1.45-1.38 (m, 2H); ¹³C NMR (125 MHz, CDCl₃) δ 72.5, 70.5, 70.1, 67.3, 61.8, 43.4, 42.6, 37.1, 31.6, 28.6; LRMS (ESI) 268.7 (M+H)⁺.

### 2-(2-(2-(Adamantan-1-yl)ethoxy)ethoxy)ethyl methanesulfonate

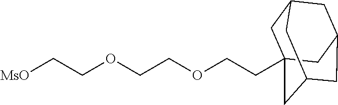 [SMILES: CS(=O)(=O)OCCOCCOCCC12CC3CC(CC(C3)C1)C2]

To a round bottom flask with stirbar and 1 mL distilled DCM was charged 2-(2-(2-(adamantan-1-yl)ethoxy)ethoxy)ethanol (50 mg, 0.18 mmol), methanesulfonyl chloride (21 μL, 0.27 mmol) and Et₃N (52 μL, 0.36 mmol). Upon stirring at room temperature for 18 h, the reaction was diluted with 2 mL 1 M aq. HCl and extracted into dichloromethane (2×5 mL). The organic layer was washed with water (2×5 mL) and dried with Na₂SO₄ to yield a crude oil. Column chromatography (5:1 to 1:1 hexanes:EtOAc) led to the isolation of 50 mg (75% yield) pure product. ¹H NMR (500 MHz, CDCl₃) δ 4.43-4.34 (m, 2H), 3.80-3.71 (m, 2H), 3.65 (dd, J=3.6, 5.6, 2H), 3.56 (dd, J=3.6, 5.6, 2H), 3.52-3.45 (m, 2H), 3.07 (s, 3H), 1.93 (s, 3H), 1.65 (dd, J=11.8, 38.7, 6H), 1.50 (d, J=1.9, 6H), 1.37 (t, J=7.6, 2H); ¹³C NMR (125 MHz, CDCl₃) δ 70.8, 70.0, 69.3, 69.0, 67.3, 43.5, 42.7, 37.7, 37.1, 31.6, 28.6; LRMS (ESI) 348.3 (M+H)⁺.

### 1-(2-(2-(2-Azidoethoxy)ethoxy)ethyl)adamantane

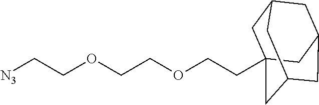 [SMILES: [N-]=[N+]=NCCOCCOCCC12CC3CC(CC(C3)C1)C2]

To a 2 dram vial with stirbar and 1 mL DMF was charged 2-(2-(2-(adamantan-1-yl)ethoxy)ethoxy)ethyl methanesulfonate (50 mg, 0.13 mmol) and sodium azide (27 mg, 0.4 mmol). Upon stirring at 80° C. for 18 h the reaction was diluted with 5 mL H₂O and extracted into EtOAc (2×5 mL). The organic layer was washed with water (2×10 mL) and dried with Na₂SO₄, and concentrated down to yield 40 mg (quant yield) of product as an oil ¹H NMR (500 MHz, CDCl₃) δ 3.65 (ddd, J=1.3, 4.0, 6.0, 4H), 3.61-3.55 (m, 2H), 3.54-3.47 (m, 2H), 3.45-3.32 (m, 2H), 1.92 (s, 3H), 1.65 (q, J=12.0, 6H), 1.50 (d, J=2.5, 6H), 1.44-1.34 (m, 2H). ¹³C NMR (75 MHz, CDCl₃) δ 70.8, 70.1, 70.0, 67.3, 50.7, 43.5, 42.6, 37.1, 31.6, 28.6; LRMS (ESI) 316.3 (M+Na)⁺.

### 2-(2-(2-(Adamantan-1-yl)ethoxy)ethoxy)ethanamine

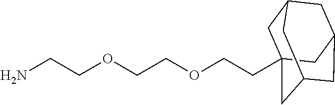 [SMILES: NCCOCCOCCC12CC3CC(CC(C3)C1)C2]

To a 2 dram vial with stirbar and 1.5 mL THF was charged 1-(2-(2-(2-azidoethoxyl)ethoxy)ethyl)adamantane (40 mg, 0.13 mmol) and triphenylphosphine (41 mg, 0.16 mmol). After stirring for 2 hours, 0.5 mL of H₂O was added and the mixture stirred for 16 h at room temperature. At this time the solvents were removed in vacuum and the mixture diluted with 5 mL 1 M aq. HCl and washed with EtOAc (2×5 mL). The aqueous layer was basified with 10 mL 3 M NaOH and the product was extracted into dichloromethane (4×10 mL). The organic layer was washed with water (2×10 mL) and dried with Na₂SO₄, and concentrated down to yield 20 mg (55% yield) of product. ¹H NMR (500 MHz, CDCl₃) δ 3.57-3.52 (m, 2H), 3.52-3.49 (m, 2H), 3.47-3.41 (m, 4H), 2.84-2.77 (m, 2H), 1.86 (s, 3H), 1.62 (d, J=12.0, 3H), 1.55 (d, J=11.2, 3H), 1.44 (d, J=2.5, 6H), 1.34 (dd, J=7.3, 15.0, 2H); ¹³C NMR (125 MHz, CDCl₃) δ 73.3, 70.4, 70.0, 67.3, 43.5, 42.7, 41.7, 37.1, 31.7, 28.7; LRMS (ESI) 268.4 (M+H)⁺.

### Example 5

### 2-(2-(4-(3-(4-Cyano-3-(trifluoromethyl)phenyl)-5,5-dimethyl-4-oxo-2-thioxoimidazolidin-1-yl)butanamido)ethoxy)ethyl 2-(adamantan-1-yl)acetate

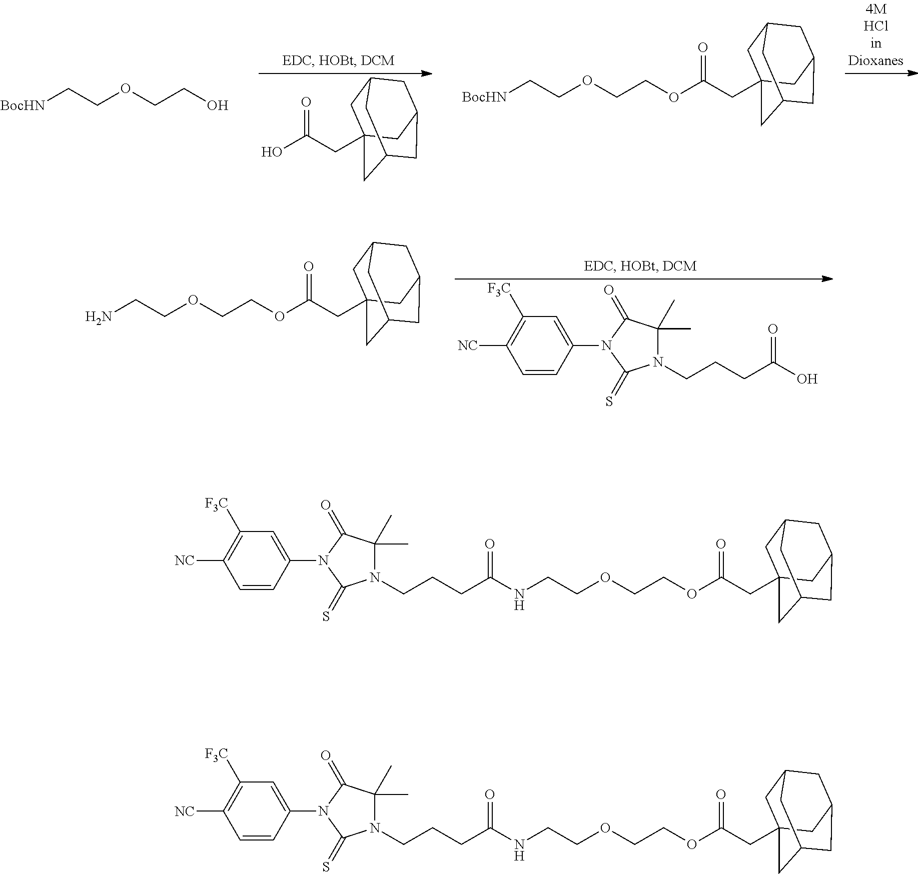

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (10.0 mg, 0.025 mmol), EDC (7.0 mg, 0.038 mmol), HOBt (6.0 mg, 0.375 mmol), and 0.35 mL dichloromethane. After 15 minutes of stirring 2-(2-aminoethoxyl)ethyl 2-(adamantan-1-yl)acetate (8.0 mg, 0.027 mmol) was added and the mixture left stir for 16 h upon which the mixture was diluted with 1 mL dichloromethane and washed with 10% aq. citric acid (2×1 mL), and saturated Na₂CO₃ (2×1 mL). The organic layer was dried with Na₂SO₄ and concentrated down to yield a crude oil which was purified by silica gel chromatography (dichloromethane to 19:1 dichloromethane:MeOH) to yield 5 mg (30% yield) of pure product as an amber oil. ¹H NMR (500 MHz, CDCl₃) δ 7.93 (d, J=8.3, 1H), 7.87 (d, J=1.7, 1H), 7.75 (dd, J=1.9, 8.3, 1H), 6.04 (t, J=5.1, 1H), 4.25-4.14 (m, 2H), 3.79-3.71 (m, 2H), 3.68-3.61 (m, 2H), 3.55 (t, J=5.0, 2H), 3.49-3.40 (m, 2H), 2.32 (t, J=6.7, 2H), 2.14 (dt, J=6.8, 14.4, 2H), 2.08 (s, 2H), 1.95 (s, 4H), 1.70-1.66 (m, 4H), 1.65-1.55 (m, 13H); ¹³C NMR (126 MHz, CDCl₃) δ 178.6, 175.5, 172.0, 171.8, 137.3, 135.3, 133.6 (q, J=33.4), 132.3, 127.2 (q, J=4.7), 121.0 (q, J=274.0), 115.0, 110.1 (q, J=2.2), 69.7 69.3, 65.4, 62.7, 49.0, 43.7, 42.5, 39.4, 36.8, 33.0, 33.0, 28.7, 23.7, 23.2; LRMS (ESI) 662.4 (M+H)⁺.

### Example 6

### N-(2-(2-(2-(Adamantan-1-yl)acetamido)ethoxy)ethyl)-4-(3-(4-cyano-3-(trifluoromethyl)phenyl)-5,5-dimethyl-4-oxo-2-thioxoimidazolidin-1-yl)butanamide

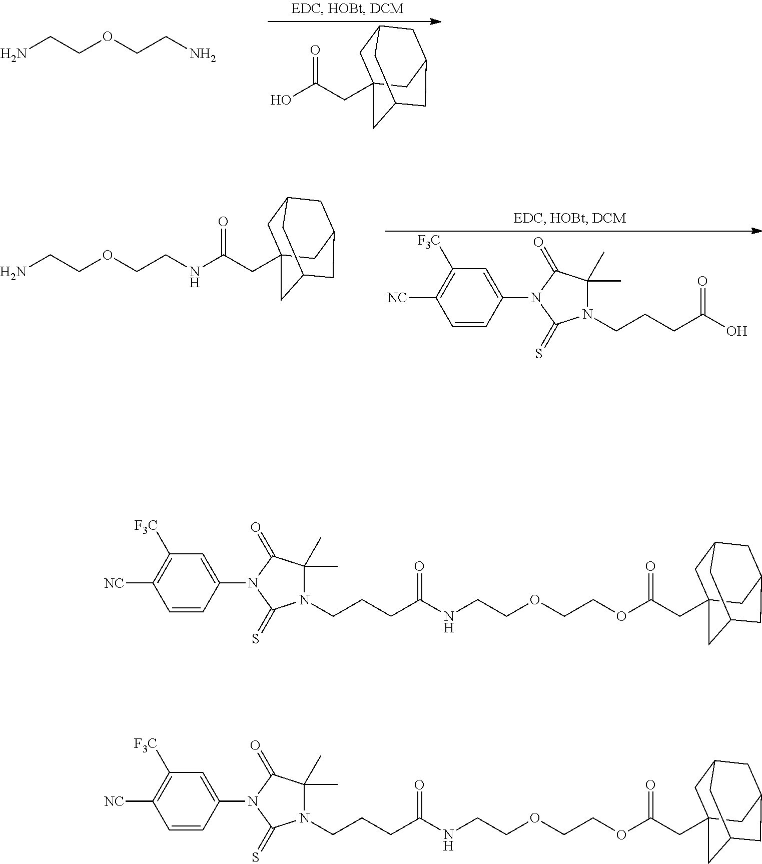 [SMILES: NCCOCCN ; O=C(O)CC12CC3CC(CC(C3)C1)C2 ; NCCOCCNC(=O)CC12CC3CC(CC(C3)C1)C2 ; CC1(C)C(=O)N(c2ccc(C#N)c(C(F)(F)F)c2)C(=S)N1CCCC(=O)O ; CC1(C)C(=O)N(c2ccc(C#N)c(C(F)(F)F)c2)C(=S)N1CCCC(=O)NCCOCCOC(=O)CC12CC3CC(CC(C3)C1)C2 ; CC1(C)C(=O)N(c2ccc(C#N)c(C(F)(F)F)c2)C(=S)N1CCCC(=O)NCCOCCOC(=O)CC12CC3CC(CC(C3)C1)C2]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (15.0 mg, 0.037 mmol), EDC (11.0 mg, 0.055 mmol), HOBt (8.5 mg, 0.55 mmol), and 0.5 mL dichloromethane. After 15 minutes of stirring, 2-(adamantan-1-yl)-N-(2-(2-aminoethoxyl)ethyl)acetamide (14 mg, 0.049 mmol) was added and the mixture left stir for 16 h upon which the mixture was diluted with 1 mL dichloromethane and washed with 10% aq. citric acid (2×1 mL), and saturated Na₂CO₃ (2×1 mL). The organic layer was dried with Na₂SO₄ and concentrated down to yield a crude oil that was purified by silica gel chromatography (dichloromethane to 19:1 dichloromethane:MeOH) to yield 12 mg (48% yield) of pure product as an amber oil. ¹H NMR (400 MHz, CDCl₃) δ 7.93 (d, J=8.3, 1H), 7.87 (d, J=1.9, 1H), 7.75 (dd, J=1.9, 8.3, 1H), 6.35 (s, 1H), 5.72 (s, 1H), 3.76 (dd, J=6.7, 9.5, 2H), 3.56-3.48 (m, 4H), 3.45-3.38 (m, 4H), 2.35 (t, J=6.7, 2H), 2.19-2.06 (m, 2H), 1.98-1.90 (m, 5H), 1.68 (d, J=12.1, 4H), 1.59 (s, 14H); ¹³C NMR (100 MHz, CDCl₃) δ 178.4, 175.3, 171.8, 171.5, 137.1, 135.1, 133.5 (q, J=33.6), 132.1, 127.1 (q, J=4.8), 121.9 (q, J=274.3), 114.9, 110.0, 70.15, 69.45, 65.33, 51.9, 43.6, 42.6, 39.4, 38.9, 36.8, 32.9, 32.8, 28.63, 23.6, 23.1; LRMS (ESI) 662.3 (M+H)⁺.

### Example 7

### 4-(3-(4-Cyano-3-(trifluoromethyl)phenyl)-5,5-dimethyl-4-oxo-2-thioxoimidazolidin-1-yl)-N-(2-(2-ethoxyethoxyl)ethyl)butanamide

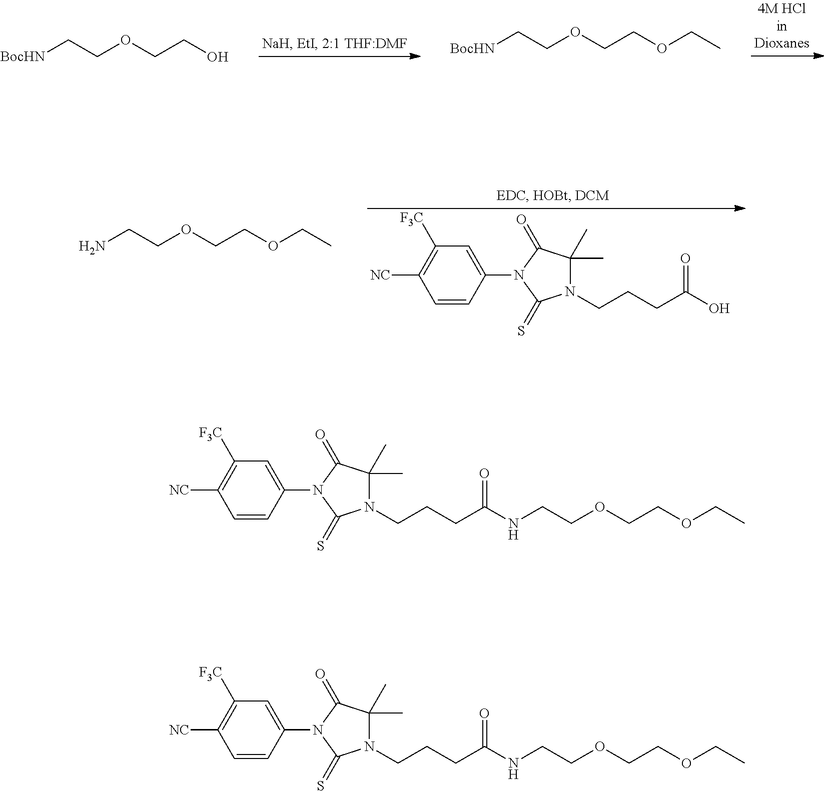

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (10.0 mg, 0.025 mmol), EDC (7.0 mg, 0.038 mmol), HOBt (6.0 mg, 0.375 mmol), and 0.35 mL dichloromethane. After 15 minutes of stirring, 2-(2-ethoxyethoxyl)ethanamine (5 mg, 0.03 mmol) was added and the mixture was stirred for 16 h, upon which the mixture was diluted with 1 mL dichloromethane and washed with 10% aq. citric acid (2×1 mL), and saturated Na₂CO₃ (2×1 mL). The organic layer was dried with Na₂SO₄ and concentrated down to yield a crude oil which was purified by silica gel chromatography (dichloromethane to 19:1 dichloromethane:MeOH) to yield 5 mg (30% yield) of pure product as an amber oil. ¹H NMR (500 MHz, CDCl₃) δ 7.88 (d, J=8.3, 1H), 7.83 (s, 1H), 7.70 (d, J=8.2, 1H), 6.14 (s, 1H), 3.77-3.66 (m, 2H), 3.56 (dd, J=3.4, 5.8, 2H), 3.52 (dd, J=3.9, 6.3, 4H), 3.50-3.44 (m, 2H), 3.42 (dd, J=5.1, 10.3, 2H), 2.31-2.22 (m, 2H), 2.15-2.02 (m, 2H), 1.55 (s, 6H), 1.16 (td, J=0.8, 7.0, 3H); ¹³C NMR (126 MHz, CDCl₃) δ 178.4, 175.4, 171.7, 137.1, 135.1, 133.5 (q, J=30.0), 132.1, 127.0 (q, J=5.0), 121.8 (q, J=278.8), 114.9, 110.0, 70.3, 69.7, 69.7, 66.7, 65.3, 43.6, 39.2, 32.89, 23.5, 23.1, 15.2; LRMS (ESI) 515.4 (M+H)⁺.

### Example 8

### N-(2-(2-(2-(2-(Adamantan-1-yloxy)ethoxy)ethoxy)ethoxy)ethyl)-4-(3-(4-cyano-3-(trifluoromethyl)phenyl)-5,5-dimethyl-4-oxo-2-thioxoimidazolidin-1-yl)butanamide

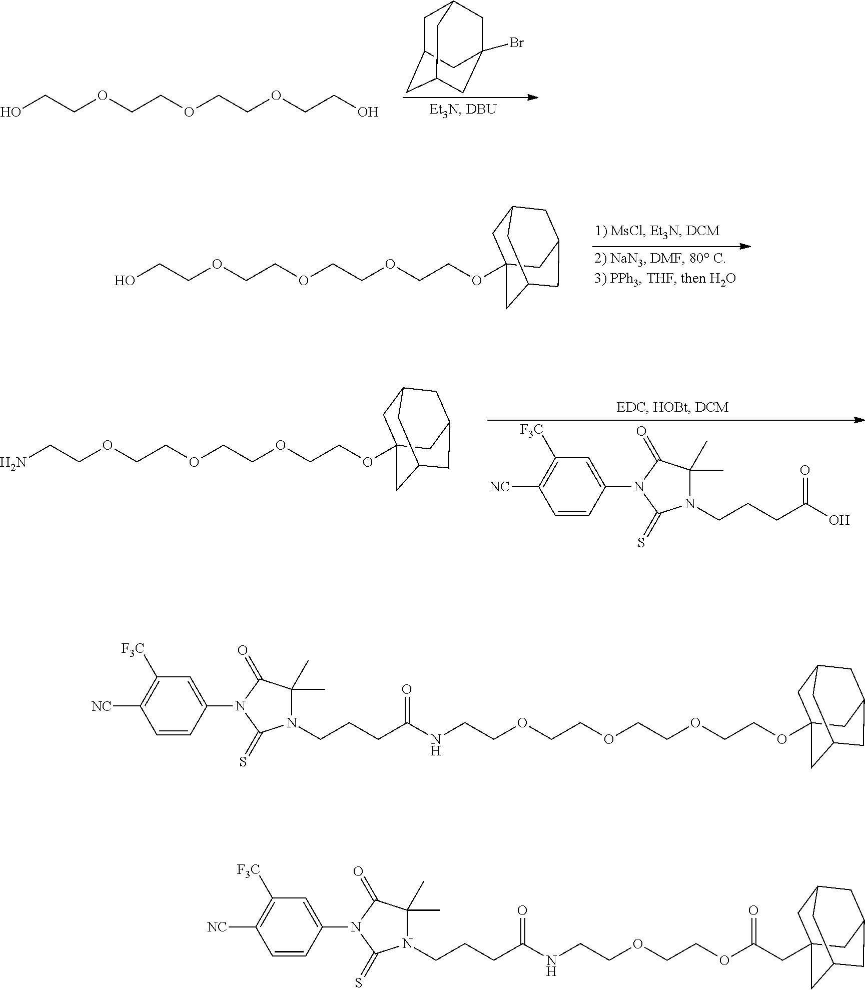

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (15.0 mg, 0.037 mmol), EDC (9.3 mg, 0.048 mmol), HOBt (7.5 mg, 0.048 mmol), and 0.35 mL dichloromethane. After 15 minutes of stirring, 2-(2-(2-(2-(adamantan-1-yloxy)ethoxy)ethoxy)ethoxy)ethanamine (13 mg, 0.040 mmol) was added and the mixture was stirred for 16 h, upon which the mixture was diluted with 1 mL dichloromethane and washed with 10% aq. citric acid (2×1 mL), and saturated Na₂CO₃ (2×1 mL). The organic layer was dried with Na₂SO₄ and concentrated down to yield a crude oil which was purified by preparative TLC (EtOAc) to yield 9 mg (34% yield) of pure product as an amber oil. ¹H NMR (500 MHz, CDCl₃) δ 7.95 (d, J=8.2, 1H), 7.91 (s, 1H), 7.78 (d, J=8.2, 1H), 7.15 (s, 1H), 3.83-3.72 (m, 2H), 3.68-3.62 (m, 8H), 3.62-3.55 (m, 6H), 3.48 (d, J=4.7, 2H), 2.36 (t, J=6.6, 2H), 2.15 (s, 5H), 1.76 (s, 6H), 1.70-1.53 (m, 12H); ¹³C NMR (126 MHz, CDCl₃) δ 178.7, 175.8, 172.3, 137.6, 135.5, 133.8 (q, J=33.2), 132.5, 127.4 (q, J=4.9), 122.4 (q, J=275.5), 115.3, 110.3, 73.0, 71.74, 71.0, 70.9, 70.8, 70.5, 70.3, 65.7, 59.6, 44.1, 41.8, 39.7, 36.8, 33.0, 30.9, 23.9, 23.5; LRMS (ESI) 709.3 (M+H)⁺.

### Example 9

### 2-(Adamantan-1-yl)-N-(2-(2-(2-(3-(4-cyano-3-(trifluoromethyl)phenyl)-5,5-dimethyl-4-oxo-2-thioxoimidazolidin-1-yl)ethoxy)ethoxy)ethyl)acetamide

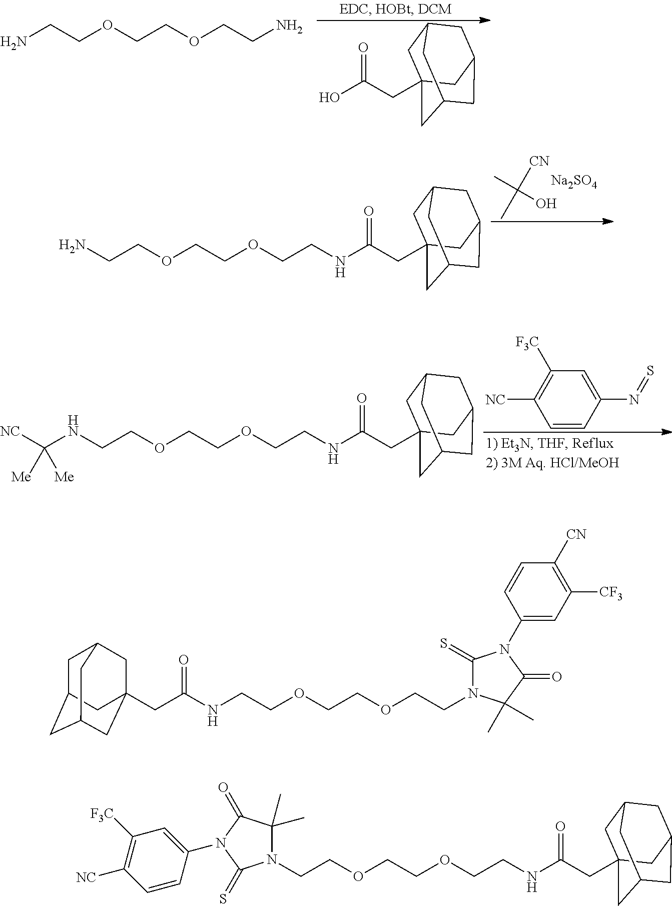

To a round bottom flask with stirbar was charged 4-cyano-3-trifluoromethyl-phenylisocyanate (55 mg, 0.20 mmol) and 2-(adamantan-1-yl)-N-(2-(2-(2-((2-cyanopropan-2-yl)amino)ethoxy)ethoxy)ethyl)acetamide (70 mg, 0.18 mmol). The mixture was dissolved with 1 mL THF, and charged with Et₃N (4.0 μL, 0.027 mmol) and stirred for 2 hours, upon which time the solvent was removed by vacuum, and the mixture ran through a silica pad to yield 53 mg of material which was dissolved in 2.5 mL of a 4:1 mixture of MeOH to 4 M HCL in dioxane. After refluxing for 2 h the reaction was cooled to room temperature and the solvent removed by vacuum. Silica gel chromatography (dichloromethane to 1:1 dichloromethane:acetone) resulted in 25 mg (41% yield) of pure product as a yellow oil. ¹H NMR (500 MHz, CDCl₃) δ 7.98 (d, J=7.2, 1H), 7.92 (s, 1H), 7.80 (s, 1H), 5.77 (s, 1H), 3.92 (d, J=18.7, 4H), 3.75-3.35 (m, 8H), 1.99 (s, 5H), 1.80-1.65 (m, 18H). ¹³C NMR (126 MHz, CDCl₃) δ 179.4, 175.7 171.5, 137.5, 135.5, 133.9 (q, J=33.8), 132.5, 127.4 (q, J=4.8), 122.3 (q, J=274.13), 115.2, 110.5, 70.9, 70.7, 70.6, 68.1, 65.7, 52.4, 44.9, 43.1, 39.6, 37.2, 33.2, 29.1, 23.7; LRMS (EST) 621.5 (M+H)⁺.

### Example 10

### (S)-4-(2-(Adamantan-1-yloxy)ethoxy)-2-methyl-N-(4-nitro-3-(trifluoromethyl)phenyl)butanamide

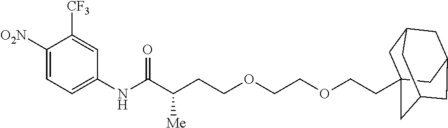 [SMILES: C[C@@H](CCOCCOCCC12CC3CC(CC(C3)C1)C2)C(=O)Nc1ccc([N+](=O)[O-])c(C(F)(F)F)c1]

To (S)-4-(2-(adamantan-1-yloxy)ethoxy)-2-methylbutanoic acid (30.0 mg, 0.1 mmol) dissolved in 0.5 mL DMA in round bottom flask was charged thionyl chloride (8.0 μL, 0.1 mmol). The mixture was stirred for 40 minutes, upon which time 4-nitro-3-trifluoromethyl aniline (21.0 mg, 0.15 mmol) was added and the reaction was stirred for 16 h. The mixture was diluted with 3 mL EtOAc and washed with 1 M HCl (2×1 mL). The organic layer was dried with Na₂SO₄ and concentrated down. The resultant oil was purified by preparative TLC (1:1 Hexanes:EtOAc) to yield 9.1 mg (20% yield) of pure product as a yellow oil. ¹H NMR (500 MHz, CDCl₃) δ 9.26 (s, 1H), 8.31 (d, J=2.0, 1H), 8.28 (dd, J=2.2, 8.9, 1H), 7.96 (d, J=8.9, 1H), 3.81 (td, J=2.3, 9.9, 1H), 3.78-3.59 (m, 2H), 3.48 (dd, J=6.2, 13.8, 1H), 3.30 (td, J=3.4, 11.2, 1H), 2.97-2.81 (m, 1H), 2.15 (s, 2H) 2.01-1.87 (m, 1H), 1.86-1.73 (m, 6H), 1.63 (dd, J=12.2, 37.2, 8H), 1.27 (d, J=6.8, 3H); ¹³C NMR (126 MHz, CDCl₃) δ 176.3, 143.6, 127.3, 125.3 (q, J=34.6), 122.7, 122.5 (q, J=274.1), 119.0 (q, J=6.0), 74.0, 71.0, 68.4, 59.8, 42.0, 38.4, 36.7, 35.6, 30.8, 17.5; LRMS (ESI) 485.3 (M+H)⁺.

### Example 11

### N-(2-(2-(2-(Adamantan-1-yl)ethoxy)ethoxy)ethyl)-4-(3-(4-cyano-3-(trifluoromethyl)phenyl)-5,5-dimethyl-4-oxo-2-thioxoimidazolidin-1-yl)butanamide

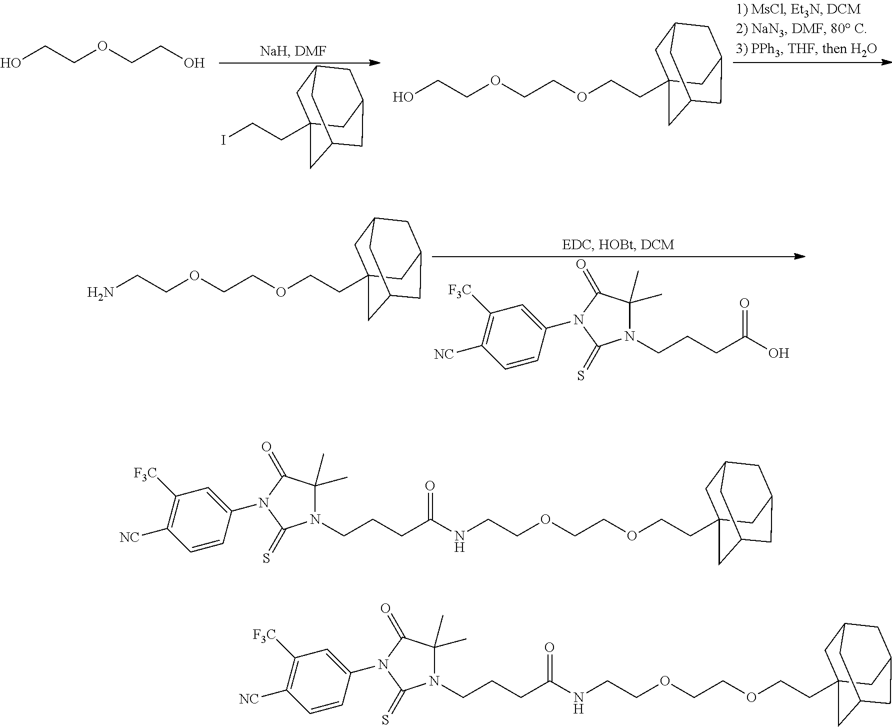

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (28.0 mg, 0.075 mmol), EDC (15 mg, 0.08 mmol), HOBt (13 mg, 0.08 mmol), and 1.0 mL dichloromethane. After 15 minutes of stirring, 2-(2-(2-(adamantan-1-yl)ethoxy)ethoxy)ethanamine (19 mg, 0.075 mmol) was added and the mixture was stirred for 16 h, upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by preparative TLC (EtOAc) to yield 19 mg (40% yield) of pure product as an amber oil. ¹H NMR (500 MHz, CDCl₃) δ 7.88 (d, J=8.3, 1H), 7.83 (d, J=1.9, 1H), 7.70 (dd, J=1.9, 8.2, 1H), 6.15 (s, 1H), 3.71 (dd, J=6.7, 9.5, 2H), 3.58-3.53 (m, 2H), 3.51 (dt, J=4.1, 8.4, 4H), 3.47-3.43 (m, 2H), 3.41 (dd, J=5.2, 10.2, 2H), 2.26 (t, J=6.7, 2H), 2.16-2.03 (m, 2H), 1.86 (s, 3H), 1.62 (t, J=13.5, 3H), 1.59 (s, 9H), 1.44 (d, J=2.3, 6H), 1.38-1.28 (m, 2H); ¹³C NMR (126 MHz, CDCl₃) δ 178.8, 175.7, 172.0, 137.5, 135.5, 133.8 (q, J=33.3), 132.5, 127.4 (q, J=4.5), 122.4 (q, J=277.3), 115.3, 110.3, 70.7, 70.4, 70.1, 67.7, 65.7, 43.4, 43.12, 39.7, 37.47, 33.3, 32.1, 29.0, 23.5; LRMS (ESI) 709.3 (M+H)⁺.

### Example 12

### Dimedones

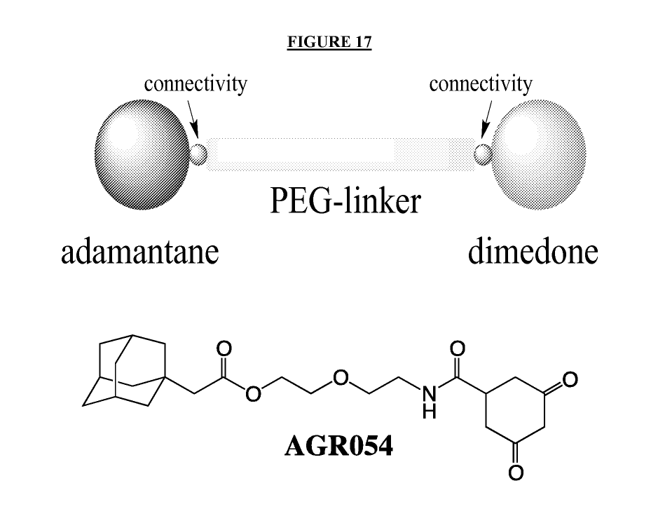

A library comprising approximately 30 dimedone compounds of varying linker lengths, hydrophobic residues and structural connectivity was prepared (FIG. 17). PEG linkers were selected because of their known stability, solubility, availability and the trivial excess to different linker lengths.

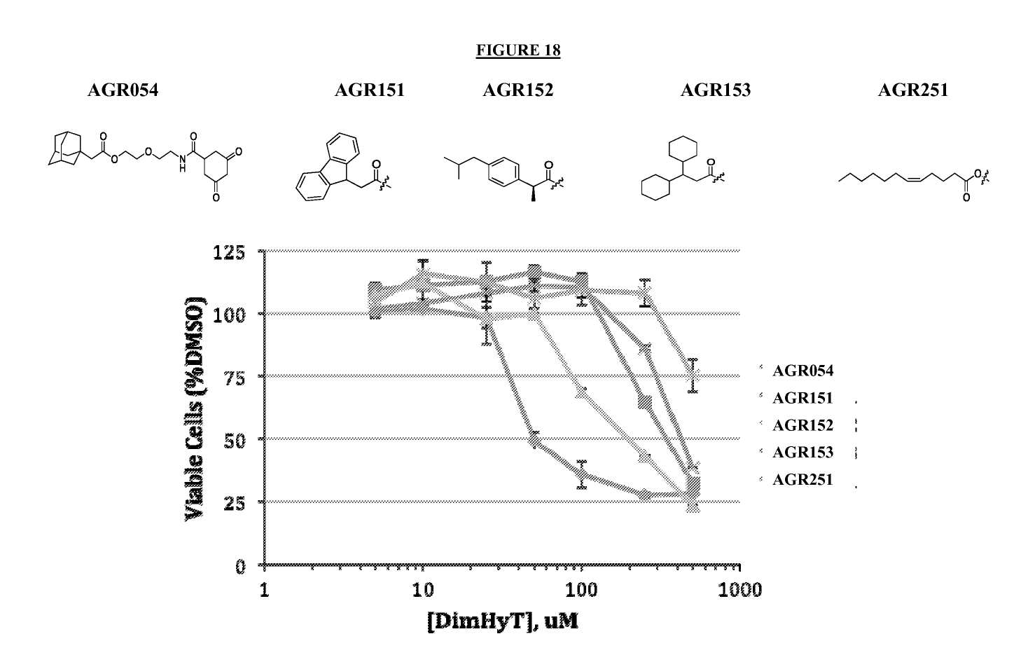

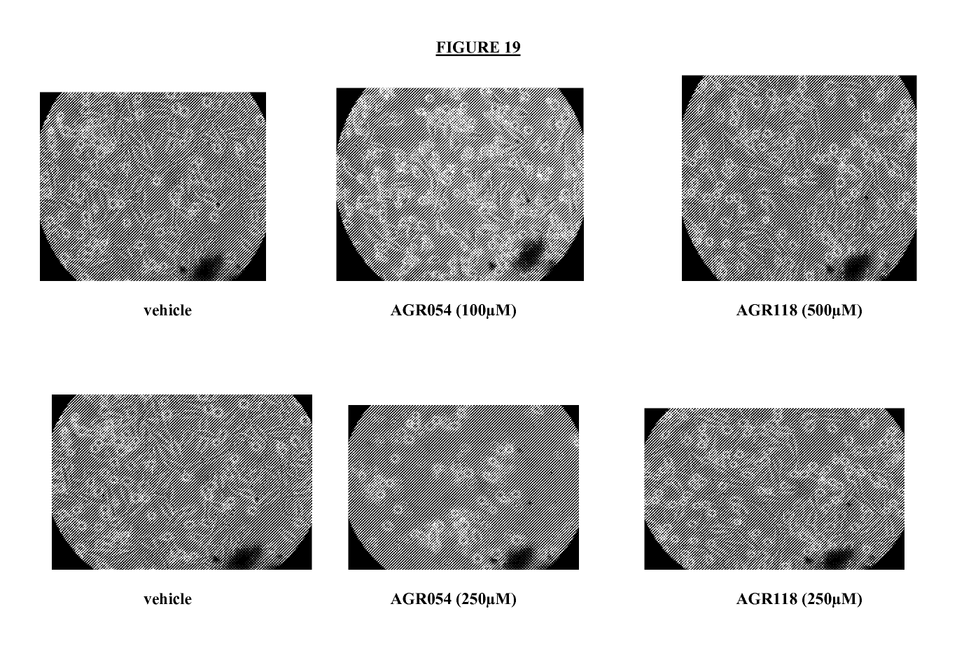

The whole library was tested in cancer cells to compare their abilities to induce intracellular protein degradation or more specifically the induction of apoptosis. A comparison of different hydrophobic head groups (FIG. 18) showed that the compound with the adamantyl group was the most active dimedone containing compound, with an IC₅₀ value of about 35 μM (assayed by Alamar Blue in HeLa cells at 24 hours). After treating HeLa cells with compound AGR054 at different concentrations for 4 hours and 8 hours, respectively, cell death was rapidly induced (FIG. 19). As a control intermediate, a less hydrophobic ethyl group was substituted for the adamantine ester head group of AGR054, and this compound was inactive.

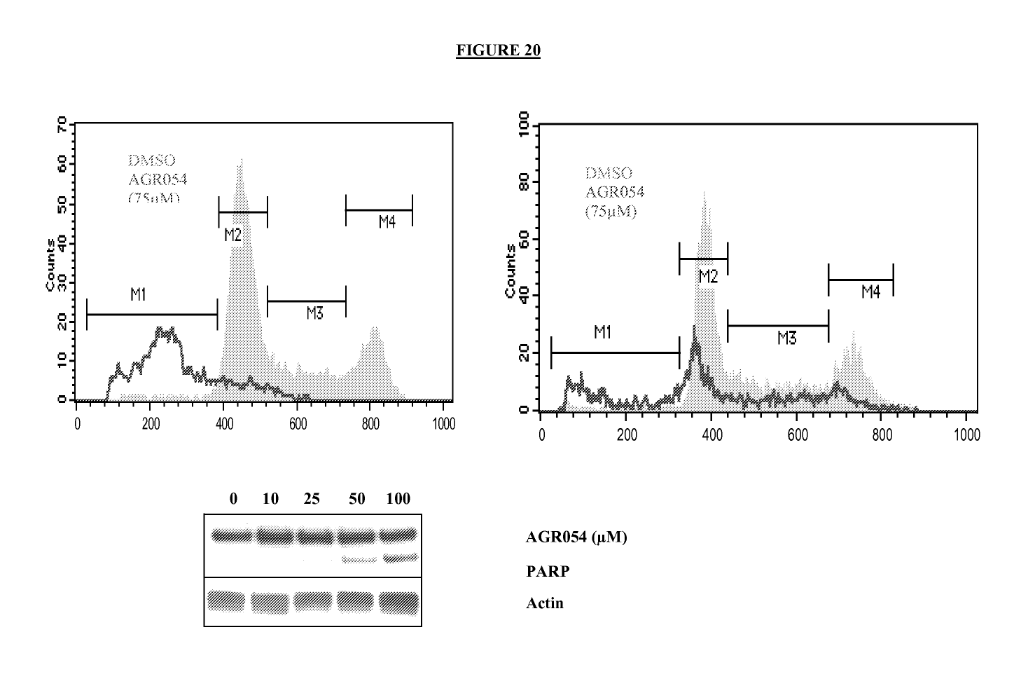

Without wishing to be bound by theory, based on the observed rapid cell death in HeLa cells after 4 and 8 hours, the cell death may have an apoptotic or a necrotic character. The cell cycle distribution was analyzed by flow cytometry to measure the DNA content. The suspected apoptotic pathway was verified by assaying for PARP (a caspase-3 substrate) cleavage by western blotting, indicating an apoptotic cell death cascade (FIG. 20, bottom row).

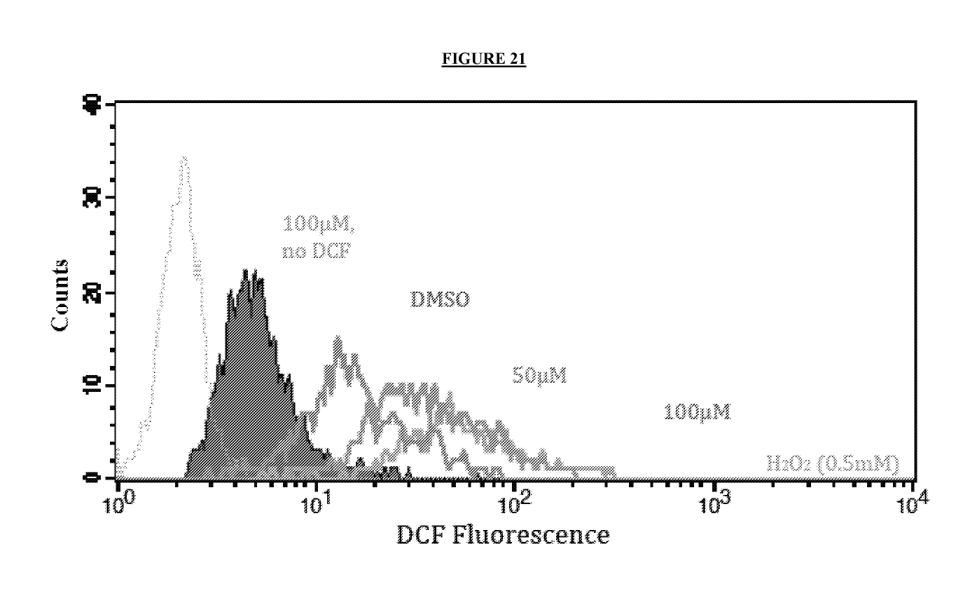

DCF-staining suggested that the apoptotic cell death cascade was initiated by a significant increase of intracellular ROS (reactive oxygen species) levels after a short treatment with AGR054 (1 hr). A concentration of 100 μM AGR054 showed a distinct shift of DCF fluorescence, which was comparable with a treatment of H₂O₂ (0.5 mM, FIG. 21).

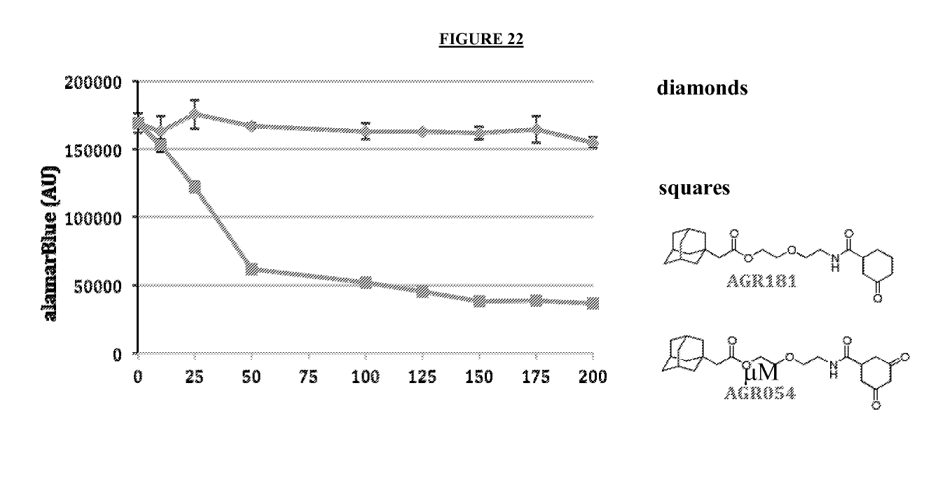

These studies indicated that a hydrophobic group is crucial for an induction of cell death (FIG. 19). In order to investigated whether the observed biological activity was also dependent of the presence of the dimedone scaffold, a control compound lacking the 1,3-diketone (AGR181) was synthesized. Treatment of cells with this compound indicated that the control compound was inactive up to a concentration of 200 μM (FIG. 22), shedding light on the activities associated with the dimedone and adamantane groups.

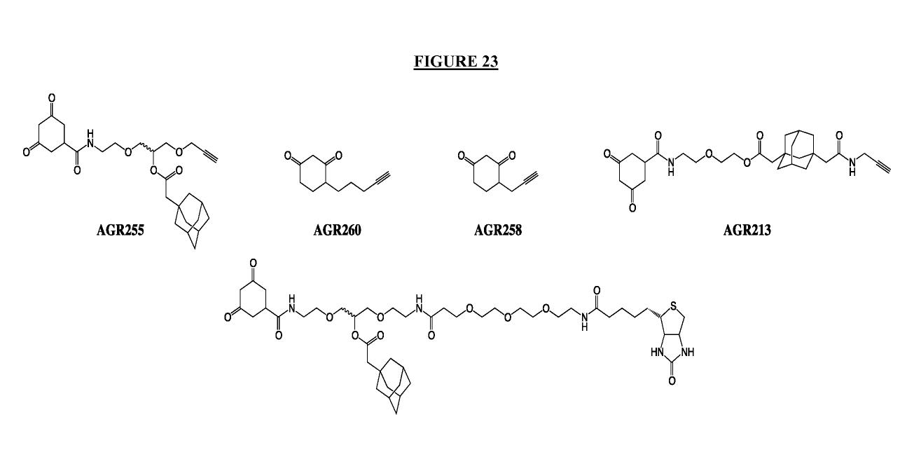

Different pull down reagents were designed and synthesized to identify the proteins labeled by AGR054 (FIG. 23).

### Example 13

### SARDS

The androgen receptor (AR) was investigated as a non-limiting target within the protein degradation approach disclosed herein. A series of selective androgen receptor degraders (SARDs) was designed based on the high affinity AR ligand RU59063 (4-[3-(4-hydroxybutyl)-4,4-dimethyl-5-oxo-2-thioxoimidazolidin-1-yl]-2-(trifluoromethyl)benzonitrile) connected via a short PEG linker to an adamantyl group.

Non-limiting examples of these compounds affected AR degradation at low micromolar concentrations. SARD 279, possessing an adamantyl group linked via an ester bond, was found to have a DC50 (half maximum degradation concentration) of 1 μM (FIG. 15A), while no degradation was detected for the parent RU 59063. Active SARDS included SARD 293, where the degron was attached via an amide bond, and SARD 033, where the degron was attached via an ether linkage, which effected degradation with DC₅₀s of about 2 μM each (FIG. 15B). There was a strong correlation between AR levels and the levels of the key downstream biomarkers for prostate cancer, i.e., prostate specific antigen (PSA). The degron group appears important for activity as SARD 280, which contained a linker but lacked the adamantly group, affected degradation only at higher concentrations and did not achieve complete AR degradation at the concentrations assayed. Additionally, racemic flutamide derivative displayed no activity under the conditions tested. These data illustrate the importance of the correct combination of ligand, linker, and hydrophobic group.

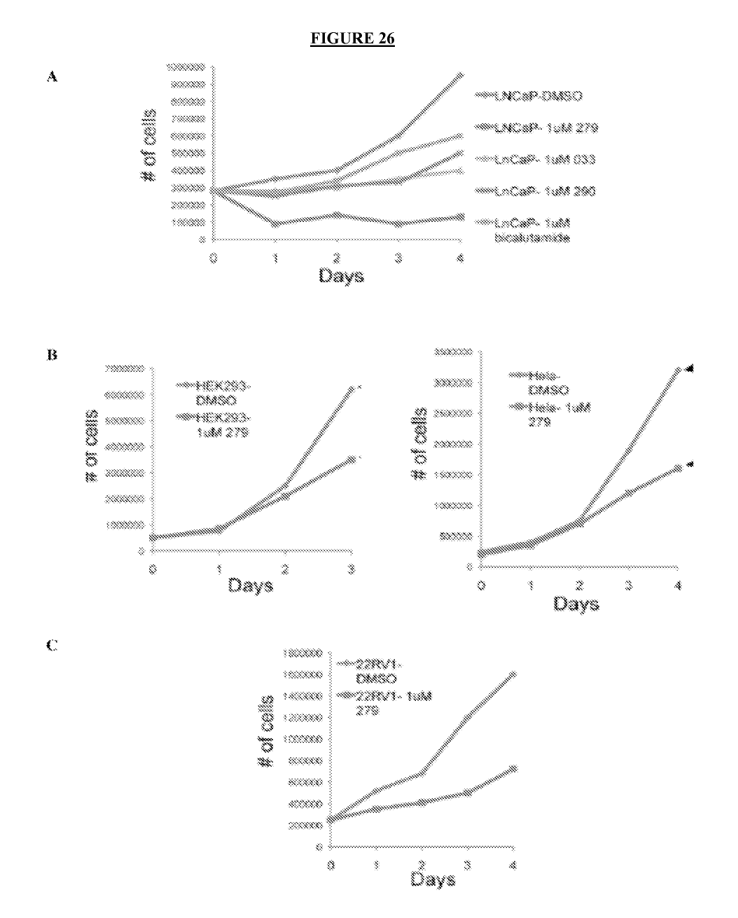

To determine whether the SARD compounds attenuate the proliferation of prostate cancer cells, androgen dependent LnCAP cells were treated with 1 μM of SARD 279, SARD 033, RU 59063 and bicalutamide (N-[4-cyano-3-(trifluoromethyl)phenyl]-3-[(4-fluorophenyl)sulfonyl]-2-hydroxy-2-methylpropanamide) for several days and cellular proliferation. Both SARD 279 and SARD 033 significantly attenuated cell growth (FIG. 26A), with SARD 279 resulting in almost complete cell death. These SARDS were more active than bicalutamide or RU 59063, suggesting that therapeutics targeting the AR for degradation represent an improved strategy for the treatment of prostate cancer.

To access general cell toxicity, AR-independent HEK293 and HELA cells were treated with 1 μM SARD 279. These cell lines did not display cytotoxic results for 48 h compared to vehicle (FIG. 26B), suggesting that SARD 279 is selectively active in AR-dependent cell lines.

SARD 279 was tested in a model of CRPC where the traditional SARMs (selective androgen receptor modulators) bicalutamide and flutamide act as agonists and thus are not effective treatments. In many castration resistant prostate tumors, AR levels are elevated 3-5 fold, thus resulting in the progression of CRPC. In one embodiment, SARDs are clinically effective in treating CRPC because they promote the removal of the AR altogether. To test this hypothesis, the androgen independent 22RV1 cell line was treated with 1 μM SARD 279. After four days, SARD 279-treated cells had attenuated proliferation compared to vehicle-treated cells, confirming a key advantage of small molecule-mediated protein knockdown over traditional small molecule inhibition.

As demonstrated herein, hydrophobic tag-based SARDS represent a novel strategy for the treatment of prostate cancer through targeted AR degradation. More generally, the hydrophobic tagging strategy should prove useful for the treatment of the myriad of diseases where traditional small molecule inhibition has come up short, or where resistance has rendered proven treatments futile.

### Example 14

### 4-(3-(4-Cyano-3-(trifluoromethyl)phenyl)-5,5-dimethyl-4-oxo-2-thioxoimidazolidin-1-yl)butanoic acid

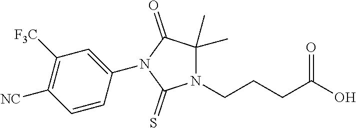 [SMILES: CC1(C)C(=O)N(c2ccc(C#N)c(C(F)(F)F)c2)C(=S)N1CCCC(=O)O]

RU59063 (145 mg, 0.38 mmol) was dissolved in 2 mL DMF, charged with PDC (1.4 g, 3.7 mmol) and stirred for 48 hours, upon which time the mixture was quenched with 10 mL 1 M HCL and extracted into Et₂O (5×25 mL). The combined organic layers were washed with brine (1×100 mL), dried with Na₂SO₄ and concentrated down to yield 135 mg (90% yield) pure product. ¹H NMR (300 MHz, CDCl₃) δ 7.94 (d, J=8.3, 1H), 7.88 (s, 1H), 7.77 (d, J=8.2, 1H), 3.82-3.65 (m, 2H), 2.50 (s, 2H), 2.14 (s, 2H), 1.59 (s, 6H); ¹³C NMR (126 MHz, CDCl₃) δ 178.6, 177.4, 175.3, 175.2, 137.1, 135.2, 133.5 (q, J=32.1), 132.1, 127.0 (q, J=4.7), 121.8 (q, J=276.2), 114.9, 110.1, 65.2, 43.3, 31.7, 23.1; LRMS (ESI) 421.2 (M+Na)⁺.

### Example 15

### 2-(2-(4-(3-(4-Cyano-3-(trifluoromethyl)phenyl)-5,5-dimethyl-4-oxo-2-thioxoimidazolidin-1-yl)butanamido)ethoxy)ethyl 2-(adamantan-1-yl)acetate (SARD 279)

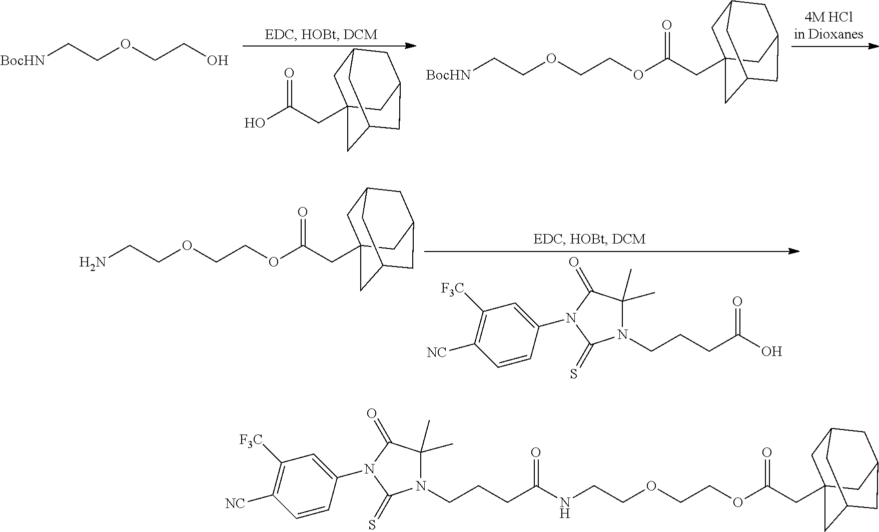

### tert-Butyl (2-(2-hydroxyethoxyl)ethyl)carbamate

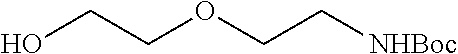 [SMILES: CC(C)(C)OC(=O)NCCOCCO]

A solution of Boc₂O (6.35 g, 30 mmol, 1.0 equiv.) in dichloromethane (35 mL) was added dropwise to a solution of 2-(2-aminoethoxy)-ethanol (3.0 mL, 30 mmol, 1.0 equiv.) in dichloromethane (20 mL) at 0° C. The reaction mixture was stirred at 0° C. for 30 min and at room temperature overnight. The reaction mixture was washed with water and the aqueous layer was extracted with dichloromethane (3×50 mL). The combined extracts were washed with brine, dried over Na₂SO₄, filtered, and concentrated under vacuum. The product (5.92 g, 99%) was isolated in high purity and was used without any further purification. ¹H NMR (400 MHz, CDCl₃) δ 5.29 (s, 1H), 3.71-3.63 (m, 2H), 3.53-3.44 (m, 4H), 3.30-3.22 (m, 2H), 3.22-3.13 (m, 1H), 1.38 (s, 1H). ¹³C NMR (100 MHz, CDCl₃) δ 156.2, 79.3, 72.3, 70.3, 61.6, 40.4, 28.4. LRMS (ESI): [M+Na]⁺ 228.3.

### 2-(2-((tert-Butoxycarbonyl)amino)ethoxy)ethyl 2-(adamantan-1-yl)acetate

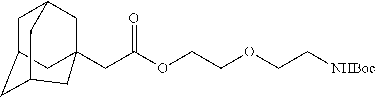 [SMILES: CC(C)(C)OC(=O)NCCOCCOC(=O)CC12CC3CC(CC(C3)C1)C2]

To a solution of 1-adamantaneacetic acid (1.00 g, 5.15 mmol, 1.0 equiv.) and tert-butyl (2-(2-hydroxyethoxyl)ethyl)carbamate (1.27 g, 6.18 mmol, 1.2 equiv.) in dichloromethane (25 mL) at room temperature was added DMAP (0.1 g, 0.82 mmol, 0.15 equiv.). The reaction mixture was cooled to 0° C. and DCC (1.27 g, 6.18 mmol, 1.2 equiv.) was added to the mixture. The resulting mixture was stirred at 0° C. for 30 min and at room temperature overnight. The reaction mixture was quenched with H₂O and the aqueous layer was extracted twice with ethyl acetate. The combined extracts were washed with brine, dried over Na₂SO₄, filtered, and concentrated. The crude product was chromatographed on silica gel and was isolated as a colorless oil (1.90 g, 97%). ¹H NMR (500 MHz, CDCl₃) δ 4.90 (s, 1H), 4.21-4.18 (m, 2H), 3.66-3.62 (m, 2H), 3.52 (t, J=5.0 Hz, 2H), 3.30 (q, J=4.9 Hz, 2H), 2.09 (s, 2H), 1.96 (s, 3H), 1.66 (dd, J=26.4, 9.6 Hz, 6H), 1.61 (s, 6H), 1.43 (s, 9H). ¹³C NMR (125 MHz, CDCl₃) δ 171.8, 79.4, 70.3, 69.1, 62.9, 48.9, 42.8, 42.5, 36.9, 36.8, 28.8, 28.7, 28.5. LRMS (ESI): [M+H]⁺ 328.2.

### 2-(2-Aminoethoxyl)ethyl 2-(adamantan-1-yl)acetate

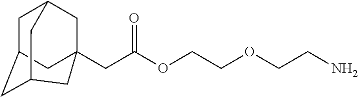 [SMILES: NCCOCCOC(=O)CC12CC3CC(CC(C3)C1)C2]

A solution of hydrogen chloride in dioxane (4M, 4 mL, 16 mmol, 3.3 equiv.) was added to 2-(2-((tert-Butoxycarbonyl)amino)ethoxy)ethyl 2-(adamantan-1-yl)acetate (1.90 g, 4.9 mmol, 1.0 equiv.) at 0° C. After stirring at 0° C. for 30 min and at room temperature for 2.0 h, the reaction mixture was concentrated. The residue was diluted with MeOH, cooled to 5° C. and treated with K₂CO₃ (1.69 g, 12.3 mmol, 2.5 equiv.). The mixture was stirred for 10 min, filtered, and evaporated. The residue was diluted with H₂O and the aqueous layer was extracted twice with ethyl acetate. The combined extracts were dried over Na₂SO₄, filtered, and concentrated. The crude product was purified by flash column chromatography on silica gel (1.32 g, 94%). ¹H NMR (500 MHz, CDCl₃) δ 6.69 (s, 2H), 4.17 (t, J=4.9 Hz, 2H), 3.71 (t, J=5.1 Hz, 2H), 3.65 (t, J=4.9 Hz, 2H), 3.12 (t, J=5.0 Hz, 2H), 2.02 (s, 2H), 1.89 (s, 3H), 1.67-1.49 (m, 12H). ¹³C NMR (125 MHz, CDCl₃) δ 171.6, 69.1, 67.2, 62.7, 48.7, 42.3, 39.6, 36.6, 32.7, 28.5. LRMS (ESI): [M+H]⁺ 282.5.

### SARD 279

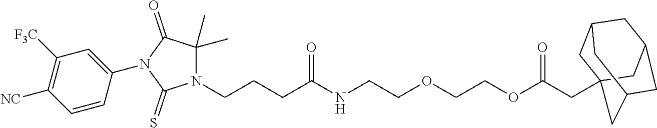 [SMILES: CC1(C)C(=O)N(c2ccc(C#N)c(C(F)(F)F)c2)C(=S)N1CCCC(=O)NCCOCCOC(=O)CC12CC3CC(CC(C3)C1)C2]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (10.0 mg, 0.025 mmol), EDC (7.0 mg, 0.038 mmol), HOBt (6.0 mg, 0.375 mmol), and 0.35 mL DCM. After 15 minutes of stirring 2-(2-Aminoethoxyl)ethyl 2-(adamantan-1-yl)acetate (8.0 mg, 0.027 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by silica gel chromatography (DCM to 19:1 DCM:MeOH) to yield 5 mg (30% yield) of pure product as an amber oil. ¹H NMR (500 MHz, CDCl₃) δ 7.93 (d, J=8.3, 1H), 7.87 (d, J=1.7, 1H), 7.75 (dd, J=1.9, 8.3, 1H), 6.04 (t, J=5.1, 1H), 4.25-4.14 (m, 2H), 3.79-3.71 (m, 2H), 3.68-3.61 (m, 2H), 3.55 (t, J=5.0, 2H), 3.49-3.40 (m, 2H), 2.32 (t, J=6.7, 2H), 2.14 (dt, J=6.8, 14.4, 2H), 2.08 (s, 2H), 1.95 (s, 4H), 1.70-1.66 (m, 4H), 1.65-1.55 (m, 13H); ¹³C NMR (126 MHz, CDCl₃) δ 178.6, 175.5, 172.0, 171.8, 137.3, 135.3, 133.6 (q, J=33.4), 132.3, 127.2 (q, J=4.7), 121.0 (q, J=274.0), 115.0, 110.1 (q, J=2.2), 69.7 69.3, 65.4, 62.7, 49.0, 43.7, 42.5, 39.4, 36.8, 33.0, 33.0, 28.7, 23.7, 23.2; LRMS (ESI) 662.4 (M+H)⁺.

### Example 16

### 4-(3-(4-Cyano-3-(trifluoromethyl)phenyl)-5,5-dimethyl-4-oxo-2-thioxoimidazolidin-1-yl)-N-(2-(2-ethoxyethoxyl)ethyl)butanamide (SARD 280)

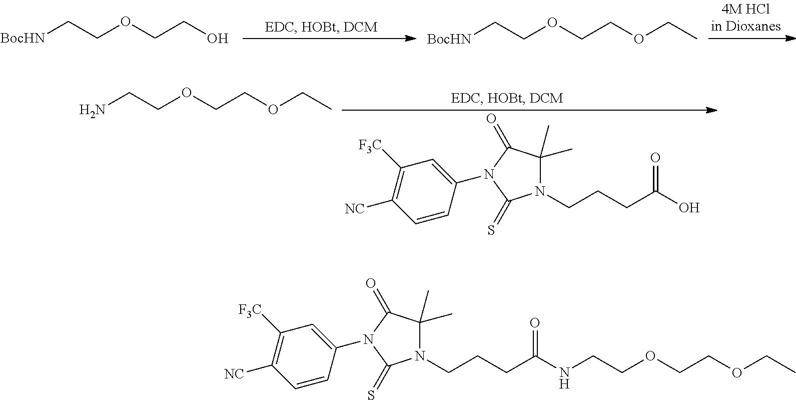

### tert-Butyl (2-(2-ethoxyethoxyl)ethyl)carbamate

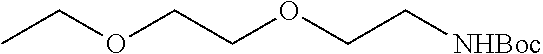 [SMILES: CCOCCOCCNC(=O)OC(C)(C)C]

To a solution of tert-Butyl (2-(2-hydroxyethoxyl)ethyl)carbamate (0.60 g, 2.9 mmol, 1.0 equiv.) in THF (24 mL) and DMF (12 mL) was added portionwise NaH (60% dispersion in mineral oil, 150 mg, 3.8 mmol, 1.3 equiv.) at 0° C. After stirring at 0° C. for 0.5 h, ethyliodine (354 μL, 4.4 mmol, 1.5 equiv.) was added to the mixture. The reaction mixture was stirred at 0° C. for 20 min and at room temperature overnight. The reaction mixture was quenched with saturated NH₄Cl solution at 0 C, extracted twice with ethyl acetate and the combined extracts were washed with brine, dried over Na₂SO₄, filtered, and concentrated. The residue was chromatographed on silica gel (250 mg, 37%). ¹H NMR (500 MHz, CDCl₃) δ 5.09 (s, 1H), 3.49-3.46 (m, 2H), 3.45-3.42 (m, 2H), 3.39 (dd, J=14.0, 6.9 Hz, 4H), 3.17 (q, J=5.1 Hz, 2H), 1.30 (s, 9H), 1.08 (t, J=7.0 Hz, 3H). ¹³C NMR (125 MHz, CDCl₃) δ 155.88, 78.8, 70.1, 70.0, 69.6, 66.5, 40.2, 28.3, 14.9. LRMS (ESI): [M+H]⁺ 234.1.

### 2-(2-Ethoxyethoxyl)ethane amine

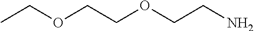

A solution of hydrogen chloride in dioxane (4M, 1 mL, 4 mmol, 4.0 equiv.) was added to tert-butyl (2-(2-ethoxyethoxyl)ethyl)carbamate (250 mg, 1.0 mmol, 1.0 equiv.) at 0° C. After stirring at 0° C. for 30 min and at room temperature for 2.0 h, the reaction mixture was concentrated. The residue was diluted with MeOH, cooled to 5° C. and treated with K₂CO₃ (1.69 g, 12.3 mmol, 2.5 equiv.). The mixture was stirred for 10 min, filtered, and evaporated. The residue was diluted with H₂O and the aqueous layer was extracted twice with ethyl acetate. The combined extracts were dried over Na₂SO₄, filtered, and concentrated. The crude product was purified by flash column chromatography on silica gel (1.30 g, 91%). ¹H NMR (500 MHz, CDCl₃) δ 7.61 (s, 2H), 3.70 (t, J=5.2 Hz, 2H), 3.54 (dd, J=5.8, 3.3 Hz, 2H), 3.46 (dd, J=5.6, 3.4 Hz, 2H), 3.39 (q, J=7.0 Hz, 2H), 3.13 (t, J=5.2 Hz, 2H), 1.05 (t, J=7.0 Hz, 3H). ¹³C NMR (125 MHz, CDCl₃) δ 70.0, 69.3, 66.6, 66.3, 39.4, 14.9. LRMS (ESI): [M+H]⁺ 134.0.

### SARD 280

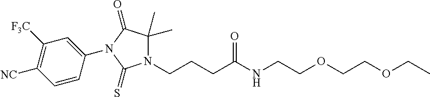 [SMILES: CCOCCOCCNC(=O)CCCN1C(=S)N(c2ccc(C#N)c(C(F)(F)F)c2)C(=O)C1(C)C]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (10.0 mg, 0.025 mmol), EDC (7.0 mg, 0.038 mmol), HOBt (6.0 mg, 0.375 mmol), and 0.35 mL DCM. After 15 minutes of stirring 2-(2-Ethoxyethoxyl)ethane amine (5 mg, 0.03 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by silica gel chromatography (DCM to 19:1 DCM:MeOH) to yield 5 mg (30% yield) of pure product as an amber oil. ¹H NMR (500 MHz, CDCl₃) δ 7.88 (d, J=8.3, 1H), 7.83 (s, 1H), 7.70 (d, J=8.2, 1H), 6.14 (s, 1H), 3.77-3.66 (m, 2H), 3.56 (dd, J=3.4, 5.8, 2H), 3.52 (dd, J=3.9, 6.3, 4H), 3.50-3.44 (m, 2H), 3.42 (dd, J=5.1, 10.3, 2H), 2.31-2.22 (m, 2H), 2.15-2.02 (m, 2H), 1.55 (s, 6H), 1.16 (td, J=0.8, 7.0, 3H); ¹³C NMR (126 MHz, CDCl₃) δ 178.4, 175.4, 171.7, 137.1, 135.1, 133.5 (q, J=30.0), 132.1, 127.0 (q, J=5.0), 121.8 (q, J=278.8), 114.9, 110.0, 70.3, 69.7, 69.7, 66.7, 65.3, 43.6, 39.2, 32.89, 23.5, 23.1, 15.2; LRMS (ESI) 515.4 (M+H)⁺.

### Example 17

### N-(2-(2-(2-(Adamantan-1-yl)acetamido)ethoxy)ethyl)-4-(3-(4-cyano-3-(trifluoromethyl)phenyl)-5,5-dimethyl-4-oxo-2-thioxoimidazolidin-1-yl)butanamide (SARD 293)

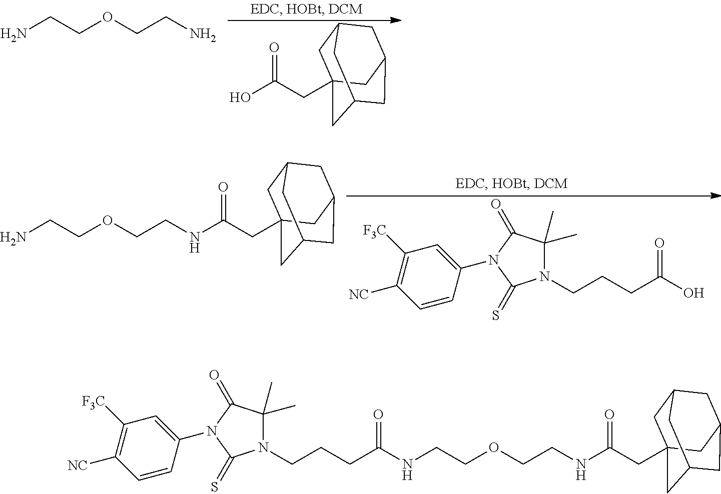

### 2-(Adamantan-1-yl)-N-(2-(2-aminoethoxyl)ethyl)acetamide

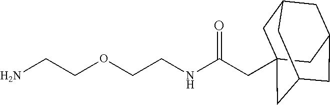 [SMILES: NCCOCCNC(=O)CC12CC3CC(CC(C3)C1)C2]

To a round bottom flask with stirbar was charged 1-adamantaneacetic acid (1.0 g, 9.7 mmol), EDC (1.43 g, 7.5 mmol), HOBt (1.16 g, 7.5 mmol), and 20 mL DCM. After 15 minutes of stirring bis(2-aminoethyl)ether (1.1 g, 10.0 mmol) was added and the mixture was stirred for 16 h upon which the mixture was diluted with 30 mL DCM and washed with saturated Na₂CO₃ (2×50 mL). The organic layer was dried with Na₂SO₄ and concentrated down to yield a crude oil that was purified by silica gel chromatography (DCM to 4:1 DCM:MeOH (0.5 N NH₃)) to yield 520 mg (35% yield) of pure product as an amber oil. ¹H NMR (300 MHz, CDCl₃) δ 3.50-3.33 (m, 8H), 2.78 (t, J=5.1, 2H), 1.95-1.85 (m, 6H), 1.65-1.45 (m, 9H); ¹³C NMR (75 MHz, CDCl₃) δ 171.1, 77.3, 72.7, 69.7, 51.4, 42.5, 38.9, 36.7, 32.6, 28.6; LRMS (ESI) 281.3 (M+H)⁺.

### SARD 293

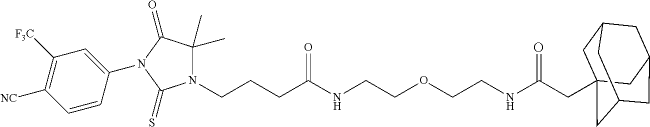 [SMILES: CC1(C)C(=O)N(c2ccc(C#N)c(C(F)(F)F)c2)C(=S)N1CCCC(=O)NCCOCCNC(=O)CC12CC3CC(CC(C3)C1)C2]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (15.0 mg, 0.037 mmol), EDC (11.0 mg, 0.055 mmol), HOBt (8.5 mg, 0.55 mmol), and 0.5 mL DCM. After 15 minutes of stirring 2-(adamantan-1-yl)-N-(2-(2-aminoethoxyl)ethyl)acetamide (14 mg, 0.049 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil that was purified by silica gel chromatography (DCM to 19:1 DCM:MeOH) to yield 12 mg (48% yield) of pure product as an amber oil. ¹H NMR (400 MHz, CDCl₃) δ 7.93 (d, J=8.3, 1H), 7.87 (d, J=1.9, 1H), 7.75 (dd, J=1.9, 8.3, 1H), 6.35 (s, 1H), 5.72 (s, 1H), 3.76 (dd, J=6.7, 9.5, 2H), 3.56-3.48 (m, 4H), 3.45-3.38 (m, 4H), 2.35 (t, J=6.7, 2H), 2.19-2.06 (m, 2H), 1.98-1.90 (m, 5H), 1.68 (d, J=12.1, 4H), 1.59 (s, 14H); ¹³C NMR (100 MHz, CDCl₃) δ 178.4, 175.3, 171.8, 171.5, 137.1, 135.1, 133.5 (q, J=33.6), 132.1, 127.1 (q, J=4.8), 121.9 (q, J=274.3), 114.9, 110.0, 70.15, 69.45, 65.33, 51.9, 43.6, 42.6, 39.4, 38.9, 36.8, 32.9, 32.8, 28.63, 23.6, 23.1; LRMS (ESI) 662.3 (M+H)⁺.

### Example 18

### 4-(3-(4-Cyano-3-(trifluoromethyl)phenyl)-5,5-dimethyl-4-oxo-2-thioxoimidazolidin-1-yl)-N-(2-(2-(hexyloxy)ethoxy)ethyl)butanamide (SARD 3-106)

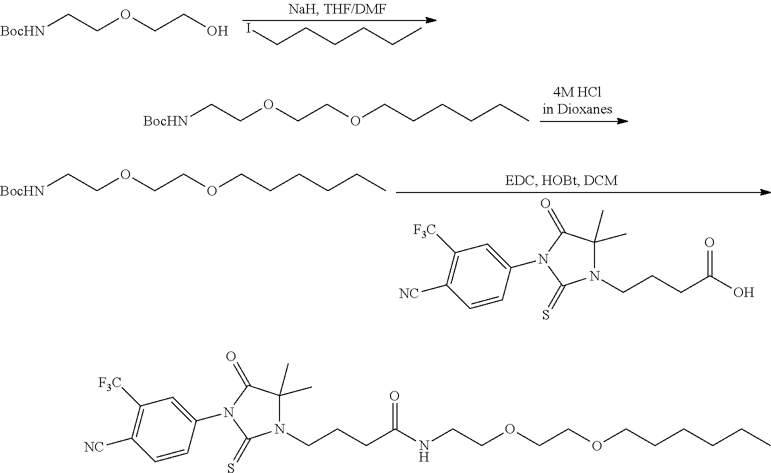

### tert-Butyl (2-(2-(hexyloxy)ethoxy)ethyl)carbamate

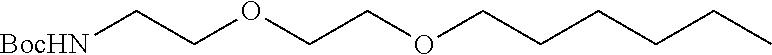 [SMILES: CCCCCCOCCOCCNC(=O)OC(C)(C)C]

To a solution of tert-butyl (2-(2-hydroxyethoxyl)ethyl)carbamate (0.30 g, 1.5 mmol, 1.0 equiv.) in THF (15 mL) and was added portionwise NaH (60% dispersion in mineral oil, 120 mg, 3.0 mmol, 2.0 equiv.) at 0° C. After stirring at 0° C. for 1 h, iodohexane (440 μL, 3.0 mmol, 2.0 equiv.) was added to the mixture. The reaction mixture was stirred at 0° C. for 20 min and at room temperature overnight. The reaction mixture was quenched with saturated NH₄Cl solution at 0° C., extracted twice with ethyl acetate and the combined extracts were washed with brine, dried over Na₂SO₄, filtered, and concentrated. The residue was chromatographed on silica gel (142 mg, 34%). ¹H NMR (500 MHz, CDCl₃) δ 5.04 (s, 1H), 3.59-3.54 (m, 2H), 3.54-3.48 (m, 4H), 3.41 (t, J=6.8 Hz, 2H), 3.27 (q, J=4.8 Hz, 2H), 1.64-1.51 (m, 2H), 1.40 (s, 9H), 1.32-1.20 (m, 6H), 0.84 (t, J=6.9 Hz, 3H). ¹³C NMR (125 MHz, CDCl₃) δ 156.1, 79.1, 71.6, 70.4, 70.3, 70.0, 40.4, 31.7, 29.6, 28.5, 25.8, 22.7, 14.1. LRMS (ESI): [M+H]⁺ 289.9.

### 2-(2-(Hexyloxy)ethoxy)ethanamine

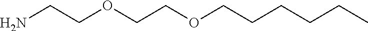

A solution of hydrogen chloride in dioxane (4M, 1 mL, 4 mmol, 8.0 equiv.) was added to tert-butyl (2-(2-(hexyloxy)ethoxy)ethyl)carbamate (142 mg, 0.5 mmol, 1.0 equiv.) at 0° C. After stirring at 0° C. for 30 min and at room temperature for 2.0 h, the reaction mixture was concentrated. The residue was diluted with MeOH, cooled to 5° C. and treated with K₂CO₃ (170 mg, 1.25 mmol, 2.5 equiv.). The mixture was stirred for 10 min, filtered, and evaporated. The residue was diluted with H₂O and the aqueous layer was extracted twice with ethyl acetate. The combined extracts were dried over Na₂SO₄, filtered, and concentrated. The crude product was purified by flash column chromatography on silica gel (93 mg, 99%). ¹H NMR (400 MHz, CD₃OD) δ 3.75-3.69 (m, 2H), 3.67 (dd, J=5.9, 2.5 Hz, 2H), 3.62 (dd, J=5.8, 2.5 Hz, 2H), 3.49 (t, J=6.7 Hz, 2H), 3.17-3.11 (m, 2H), 1.66-1.53 (m, 2H), 1.41-1.26 (m, 6H), 0.91 (t, J=6.8 Hz, 3H). ¹³C NMR (100 MHz, CD₃OD) δ 72.5, 71.3, 71.1, 67.8, 40.7, 32.8, 30.6, 26.8, 23.6, 14.4. LRMS (ESI): [M+H]⁺ 190.3.

### SARD 3-106

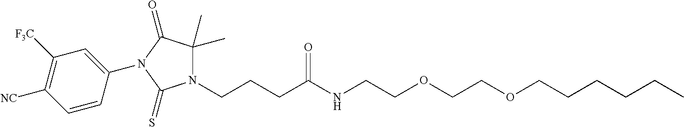 [SMILES: CCCCCCOCCOCCNC(=O)CCCN1C(=S)N(c2ccc(C#N)c(C(F)(F)F)c2)C(=O)C1(C)C]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (43.0 mg, 0.11 mmol), EDC (20.0 mg, 0.12 mmol), HOBt (17 mg, 0.12 mmol), and 0.5 mL DCM. After 15 minutes of stirring, 2-(2-(hexyloxy)ethoxy)ethanamine (28 mg, 0.11 mmol) was added and the mixture was stirred for 16 h, upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil that was purified by silica gel chromatography (2:1 Hex:EtOAC to 100% EtOAc) to yield 24 mg (40% yield) of pure product as an amber oil. ¹H NMR (500 MHz, CDCl₃) δ 7.88 (d, J=8.3, 1H), 7.83 (d, J=1.9, 1H), 7.70 (dd, J=1.9, 8.2, 1H), 6.18 (s, 1H), 3.77-3.63 (m, 2H), 3.60-3.47 (m, 6H), 3.40 (dt, J=6.1, 10.4, 4H), 2.26 (t, J=6.7, 2H), 2.08 (dt, J=6.8, 14.5, 2H), 1.60-1.41 (m, 8H), 1.32-1.12 (m, 6H), 0.87-0.69 (m, 3H). ¹³C NMR (125 MHz, CDCl₃) δ 178.8, 175.7, 172.0, 137.6, 135.5, 133.8 (q, J=33.8), 132.5, 127.4 (q, J=4.6), 122.3 (q, J=272.3), 115.2 110.4, 72.0, 70.7, 70.4, 70.1, 65.7, 44.0, 39.7, 33.3, 32.1, 30.0, 26.2, 23.94, 23.5, 23.0, 14.4; LRMS (ESI) 571.8 (M+H)⁺.

### Example 19

### N-(2-(2-(2-(2-(Adamantan-1-yloxy)ethoxy)ethoxy)ethoxy)ethyl)-4-(3-(4-cyano-3-(trifluoromethyl)phenyl)-5,5-dimethyl-4-oxo-2-thioxoimidazolidin-1-yl)butanamide (SARD 3-033)

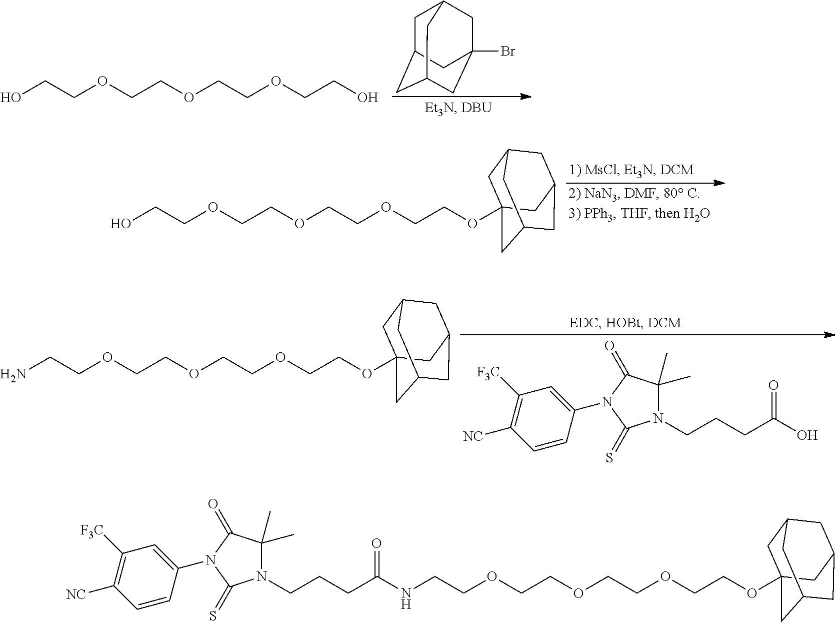

### 2-(2-(2-(2-(Adamantan-1-yloxy)ethoxy)ethoxy)ethoxy)ethanol

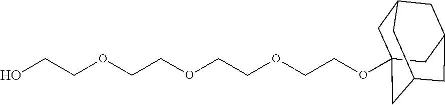 [SMILES: OCCOCCOCCOCCOC12CC3CC(CC(C3)C1)C2]

To a round bottom flask with stirbar was charged 2,2′-((oxybis(ethane-2,1-diyl))bis(oxy))diethanol (4.5 g, 23.3 mmol), 1-bromoadamantane (1.0 g, 4.6 mmol), Et₃N (2.1 Ml, 15.0 mmol) and DBU (0.033 mL, 0.23 mmol). Upon stirring at 110° C. for 18 h the reaction was diluted with 25 mL 1 M Aq. HCl and extracted in to DCM (2×25 mL). The organic layer was washed with water (2×25 mL) and dried with Na₂SO₄ to yield a crude oil. Column chromatography (4:1 Hex:EtOAc to 100% EtOAc) led to the isolation of 202 mg (30% yield) pure product. ¹H NMR (500 MHz, CDCl₃) δ 3.69-3.63 (m, 2H), 3.62-3.57 (m, 8H), 3.56-3.46 (m, 6H), 3.30 (s, 1H), 2.07 (s, 3H), 1.76-1.62 (m, 6H), 1.54 (q, J=12.2, 6H); ¹³C NMR (126 MHz, CDCl₃) δ 73.0, 72.6, 71.5, 70.9, 70.9, 70.9, 70.6, 61.9, 59.6, 41.7, 36.8, 30.8; LRMS (ESI) 329.5 (M+H)⁺.

### 2-(2-(2-(2-(Adamantan-1-yloxy)ethoxy)ethoxy)ethoxy)ethyl methanesulfonate

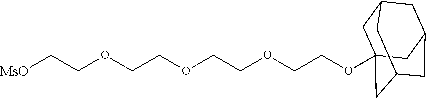 [SMILES: CS(=O)(=O)OCCOCCOCCOCCOC12CC3CC(CC(C3)C1)C2]

To a round bottom flask with stirbar and 5 mL distilled DCM was charged 2-(2-(2-(2-(adamantan-1-yloxy)ethoxy)ethoxy)ethoxy)ethanol (200 mg, 0.6 mmol), methanesulfonyl chloride (70 μL, 0.9 mmol) and Et₃N (253.0 μL, 1.8 mmol). Upon stirring at rt for 18 h the reaction was diluted with 5 mL 1 M Aq. HCl and extracted into DCM (2×5 mL). The organic layer was washed with water (2×10 mL) and dried with Na₂SO₄ to yield a crude oil. Column chromatography (3:1 Hex:EtOAc to 100% EtOAc) led to the isolation of 200 mg (82% yield) pure product. ¹H NMR (300 MHz, CDCl₃) δ 4.43-4.32 (m, 2H), 3.82-3.71 (m, 2H), 3.70-3.61 (m, 8H), 3.57 (s, 2H), 3.08-3.06 (m, 2H), 2.13 (s, 3H), 1.73 (m, 6H), 1.68-1.51 (m, 6H)¹³C NMR (75 MHz, CDCl₃) δ 72.3, 71.3, 70.6, 70.6, 70.5, 70.5, 69.3, 69.0, 59.2, 41.4, 39.4, 37.3, 30.5; LRMS (ESI) 405.8 (M+H)⁺.

### 1-(2 (2 (2 (2 Azidoethoxy)ethoxy)ethoxy)ethoxy)adamantane

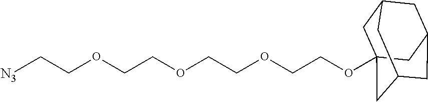 [SMILES: [N-]=[N+]=NCCOCCOCCOCCOC12CC3CC(CC(C3)C1)C2]

To a 2 dram vial with stirbar and 3 mL DMF was charged 2-(2-(2-(2-(adamantan-1-yloxy)ethoxy)ethoxy)ethoxy)ethyl methanesulfonate (300 mg, 0.74 mmol) and sodium azide (120 mg, 1.85 mmol). Upon stirring at 80° C. for 18 h the reaction was diluted with 5 mL H₂O and extracted into EtOAc (2×5 mL). The organic layer was washed with water (2×10 mL) and dried with Na₂SO₄, and concentrated down to yield a crude oil. Column chromatography (DCM to 10:1 DCM:MeOH) resulted in the recovery of 172 mg (63% yield) of pure product. ¹H NMR (500 MHz, CDCl₃) δ 3.66-3.58 (m, 11H), 3.54 (s, 3H), 3.34 (s, 2H), 2.09 (s, 3H), 1.69 (s, 6H), 1.56 (q, J=12.0, 6H). ¹³C NMR (125 MHz, CDCl₃) δ 72.2, 71.2, 70.7, 70.6, 70.6, 70.5, 70.0, 59.2, 50.6, 41.4, 36.4, 30.4; LRMS (ESI) 326.6 (M-N₂).

### 2-(2-(2-(2-(Adamantan-1-yloxy)ethoxy)ethoxy)ethoxy)ethanamine

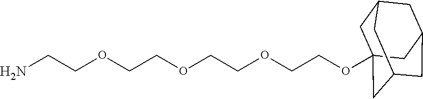 [SMILES: NCCOCCOCCOCCOC12CC3CC(CC(C3)C1)C2]

To a 2 dram vial with stirbar and 2 mL THF was charged 1-(2-(2-(2-(2-azidoethoxyl)ethoxy)ethoxy)ethoxy)adamantane (170 mg, 0.47 mmol) and triphenylphosphine (150 mg, 0.57 mmol). 20 μL of H₂O was added after 2 h and the mixture let stir for 16 h at rt. The solvents were removed via rotovap and the mixture diluted with 5 mL 1 M aq. HCl and washed with EtOAc (2×5 mL). The aqueous layer was basified with 20 mL 3 M NaOH and the product was extracted in to DCM (4×25 mL). The organic layer was washed with water (2×10 mL) and dried with Na₂SO₄, and concentrated down to yield 85 mg (55% yield) of product. ¹H NMR (500 MHz, CDCl₃) δ 3.70-3.56 (m, 8H), 3.54 (s, 4H), 3.48 (t, J=4.9, 2H), 2.83 (s, 2H), 2.09 (s, 3H), 1.70 (s, 6H), 1.56 (q, J=12.3, 6H); ¹³C NMR (125 MHz, CDCl₃) δ 73.0, 72.3, 71.2, 70.5, 70.5, 70.5, 70.2, 59.2, 41.6, 41.4, 36.4, 30.4; LRMS (ESI) 327.4 (M+H)⁺.

### SARD 3-033

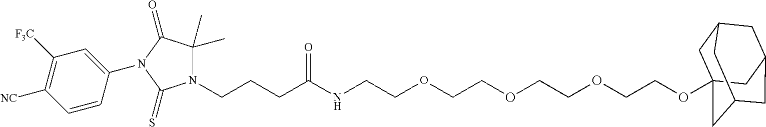 [SMILES: CC1(C)C(=O)N(c2ccc(C#N)c(C(F)(F)F)c2)C(=S)N1CCCC(=O)NCCOCCOCCOCCOC12CC3CC(CC(C3)C1)C2]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (15.0 mg, 0.037 mmol), EDC (9.3 mg, 0.048 mmol), HOBt (7.5 mg, 0.048 mmol), and 0.35 mL DCM. After 15 minutes of stirring 2-(2-(2-(2-(adamantan-1-yloxy)ethoxy)ethoxy)ethoxy)ethanamine (13 mg, 0.040 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by preparative TLC (EtOAc) to yield 9 mg (34% yield) of pure product as an amber oil. ¹H NMR (500 MHz, CDCl₃) δ 7.95 (d, J=8.2, 1H), 7.91 (s, 1H), 7.78 (d, J=8.2, 1H), 7.15 (s, 1H), 3.83-3.72 (m, 2H), 3.68-3.62 (m, 8H), 3.62-3.55 (m, 6H), 3.48 (d, J=4.7, 2H), 2.36 (t, J=6.6, 2H), 2.15 (s, 5H), 1.76 (s, 6H), 1.70-1.53 (m, 12H); ¹³C NMR (126 MHz, CDCl₃) δ 178.7, 175.8, 172.3, 137.6, 135.5, 133.8 (q, J=33.2), 132.5, 127.4 (q, J=4.9), 122.4 (q, J=275.5), 115.3, 110.3, 73.0, 71.74, 71.0, 70.9, 70.8, 70.5, 70.3, 65.7, 59.6, 44.1, 41.8, 39.7, 36.8, 33.0, 30.9, 23.9, 23.5; LRMS (ESI) 709.3 (M+H)⁺.

### Example 20

### N-(2-(2-(2-(Adamantan-1-yl)ethoxy)ethoxy)ethyl)-4-(3-(4-cyano-3-(trifluoromethyl)phenyl)-5,5-dimethyl-4-oxo-2-thioxoimidazolidin-1-yl)butanamide (SARD 3-126)

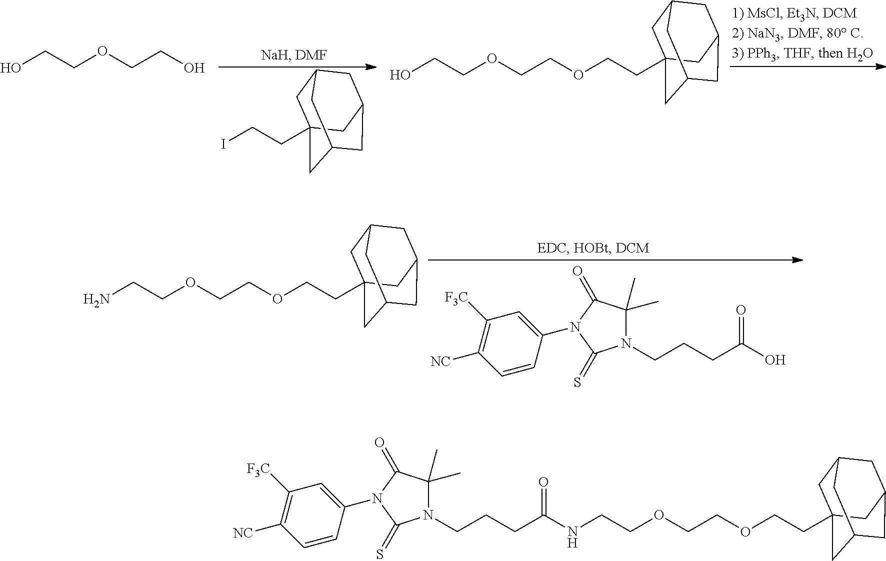 [SMILES: OCCOCCO ; ICCC12CC3CC(CC(C3)C1)C2 ; OCCOCCOCCC12CC3CC(CC(C3)C1)C2 ; NCCOCCOCCC12CC3CC(CC(C3)C1)C2 ; CC1(C)C(=O)N(c2ccc(C#N)c(C(F)(F)F)c2)C(=S)N1CCCC(=O)O ; CC1(C)C(=O)N(c2ccc(C#N)c(C(F)(F)F)c2)C(=S)N1CCCC(=O)NCCOCCOCCC12CC3CC(CC(C3)C1)C2]

### 2-(2-(2-(Adamantan-1-yl)ethoxy)ethoxy)ethanol

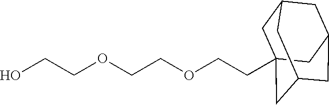 [SMILES: OCCOCCOCCC12CC3CC(CC(C3)C1)C2]

To a round bottom flask with stirbar was charged 60% NaH (400 mg, 10.0 mmol) which was then sparged with argon and suspended in dry DMF (20 mL) and cooled to 0° C. The bis(2-hydroxyethyl)ether (0.5 g, 5.0 mmol) was then added and the mixture let stir 45 minutes. Upon this time the 1-(2-iodoethyl)adamantane (300 mg, 1.05 mmol) was added and the reaction left warm to room temperature and stir for 18 h. The reaction was quenched with 25 mL saturated NH₄Cl and extracted into EtOAc (3×25 mL). The organic layer was then washed with Brine (3×30 mL) and concentrated down to yield a crude oil which was purified by silica gel chromatography (5:1 to 1:1 Hexanes:EtOAc to yield 55 mg (20% yield) of pure product ¹H NMR (500 MHz, CDCl₃) δ 3.79-3.72 (m, 2H), 3.68 (dd, J=3.7, 5.6, 2H), 3.65-3.62 (m, 2H), 3.59 (dd, J=3.7, 5.6, 2H), 3.53 (dd, J=5.2, 10.1, 2H), 1.94 (s, 3H), 1.70 (d, J=12.1, 3H), 1.63 (d, J=10.8, 6H), 1.52 (d, J=2.4, 6H), 1.45-1.38 (m, 2H); ¹³C NMR (125 MHz, CDCl₃) δ 72.5, 70.5, 70.1, 67.3, 61.8, 43.4, 42.6, 37.1, 31.6, 28.6; LRMS (ESI) 268.7 (M+H)⁺.

### 2-(2-(2-(Adamantan-1-yl)ethoxy)ethoxy)ethyl methanesulfonate

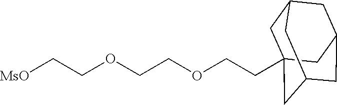 [SMILES: CS(=O)(=O)OCCOCCOCCC12CC3CC(CC(C3)C1)C2]

To a round bottom flask with stirbar and 1 mL distilled DCM was charged 2-(2-(2-(adamantan-1-yl)ethoxy)ethoxy)ethanol (50 mg, 0.18 mmol), methanesulfonyl chloride (21 μL, 0.27 mmol) and Et₃N (52 μL, 0.36 mmol). Upon stirring at rt for 18 h the reaction was diluted with 2 mL 1 M Aq. HCl and extracted in to DCM (2×5 mL). The organic layer was washed with water (2×5 mL) and dried with Na₂SO₄ to yield a crude oil. Column chromatography (5:1 to 1:1 Hex:EtOAc) led to the isolation of 50 mg (75% yield) pure product. ¹H NMR (500 MHz, CDCl₃) δ 4.43-4.34 (m, 2H), 3.80-3.71 (m, 2H), 3.65 (dd, J=3.6, 5.6, 2H), 3.56 (dd, J=3.6, 5.6, 2H), 3.52-3.45 (m, 2H), 3.07 (s, 3H), 1.93 (s, 3H), 1.65 (dd, J=11.8, 38.7, 6H), 1.50 (d, J=1.9, 6H), 1.37 (t, J=7.6, 2H); ¹³C NMR (125 MHz, CDCl₃) δ 70.8, 70.0, 69.3, 69.0, 67.3, 43.5, 42.7, 37.7, 37.1, 31.6, 28.6; LRMS (ESI) 348.3 (M+H)⁺.

### 1-(2-(2-(2-Azidoethoxy)ethoxy)ethyl)adamantane

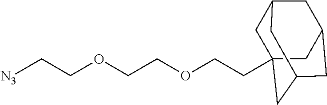 [SMILES: [N-]=[N+]=NCCOCCOCCC12CC3CC(CC(C3)C1)C2]

To a 2 dram vial with stirbar and 1 mL DMF was charged mesylate (50 mg, 0.13 mmol) and sodium azide (27 mg, 0.4 mmol). Upon stirring at 80° C. for 18 h the reaction was diluted with 5 mL H₂O and extracted into EtOAc (2×5 mL). The organic layer was washed with water (2×10 mL) and dried with Na₂SO₄, and concentrated down to yield 40 mg (quant yield) of product as an oil ¹H NMR (500 MHz, CDCl₃) δ 3.65 (ddd, J=1.3, 4.0, 6.0, 4H), 3.61-3.55 (m, 2H), 3.54-3.47 (m, 2H), 3.45-3.32 (m, 2H), 1.92 (s, 3H), 1.65 (q, J=12.0, 6H), 1.50 (d, J=2.5, 6H), 1.44-1.34 (m, 2H). ¹³C NMR (75 MHz, CDCl₃) δ 70.8, 70.1, 70.0, 67.3, 50.7, 43.5, 42.6, 37.1, 31.6, 28.6; LRMS (ESI) 316.3 (M+Na)⁺.

### 2-(2-(2-(Adamantan-1-yl)ethoxy)ethoxy)ethanamine

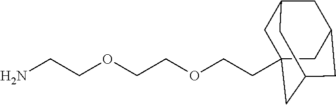 [SMILES: NCCOCCOCCC12CC3CC(CC(C3)C1)C2]

To a 2 dram vial with stirbar and 1.5 mL THF was charged 1-(2-(2-(2-azidoethoxyl)ethoxy)ethyl)adamantane (40 mg, 0.13 mmol) and triphenylphosphine (41 mg, 0.16 mmol). After stirring for 2 hours, 0.5 mL of H₂O was added and the mixture let stir for 16 h at rt. At this time the solvents were removed via vacuum and the mixture diluted with 5 mL 1 M aq. HCl and washed with EtOAc (2×5 mL). The aqueous layer was basified with 10 mL 3 M NaOH and the product was extracted into DCM (4×10 mL). The organic layer was washed with water (2×10 mL) and dried with Na₂SO₄, and concentrated down to yield 20 mg (55% yield) of product. ¹H NMR (500 MHz, CDCl₃) δ 3.57-3.52 (m, 2H), 3.52-3.49 (m, 2H), 3.47-3.41 (m, 4H), 2.84-2.77 (m, 2H), 1.86 (s, 3H), 1.62 (d, J=12.0, 3H), 1.55 (d, J=11.2, 3H), 1.44 (d, J=2.5, 6H), 1.34 (dd, J=7.3, 15.0, 2H); ¹³C NMR (125 MHz, CDCl₃) δ 73.3, 70.4, 70.0, 67.3, 43.5, 42.7, 41.7, 37.1, 31.7, 28.7; LRMS (ESI) 268.4 (M+H)⁺.

### SARD 3-126

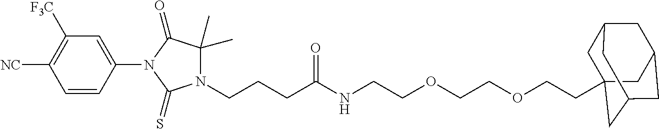 [SMILES: CC1(C)C(=O)N(c2ccc(C#N)c(C(F)(F)F)c2)C(=S)N1CCCC(=O)NCCOCCOCCC12CC3CC(CC(C3)C1)C2]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (28.0 mg, 0.075 mmol), EDC (15 mg, 0.08 mmol), HOBt (13 mg, 0.08 mmol), and 1.0 mL DCM. After 15 minutes of stirring 2-(2-(2-(adamantan-1-yl)ethoxy)ethoxy) ethanamine (19 mg, 0.075 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by preparative TLC (EtOAc) to yield 19 mg (40% yield) of pure product as an amber oil. ¹H NMR (500 MHz, CDCl₃) δ 7.88 (d, J=8.3, 1H), 7.83 (d, J=1.9, 1H), 7.70 (dd, J=1.9, 8.2, 1H), 6.15 (s, 1H), 3.71 (dd, J=6.7, 9.5, 2H), 3.58-3.53 (m, 2H), 3.51 (dt, J=4.1, 8.4, 4H), 3.47-3.43 (m, 2H), 3.41 (dd, J=5.2, 10.2, 2H), 2.26 (t, J=6.7, 2H), 2.16-2.03 (m, 2H), 1.86 (s, 3H), 1.62 (t, J=13.5, 3H), 1.59 (s, 9H), 1.44 (d, J=2.3, 6H), 1.38-1.28 (m, 2H); ¹³C NMR (126 MHz, CDCl₃) δ 178.8, 175.7, 172.0, 137.5, 135.5, 133.8 (q, J=33.3), 132.5, 127.4 (q, J=4.5), 122.4 (q, J=277.3), 115.3, 110.3, 70.7, 70.4, 70.1, 67.7, 65.7, 43.4, 43.12, 39.7, 37.47, 33.3, 32.1, 29.0, 23.5; LRMS (ESI) 709.3 (M+H)⁺.

### Example 21

### N-(2-(2-(2-(4-(tert-Butyl)phenoxy)ethoxy)ethoxy)ethyl)-4-(3-(4-cyano-3-(trifluoromethyl)phenyl)-5,5-dimethyl-4-oxo-2-thioxoimidazolidin-1-yl) butanamide (SARD 3-190)

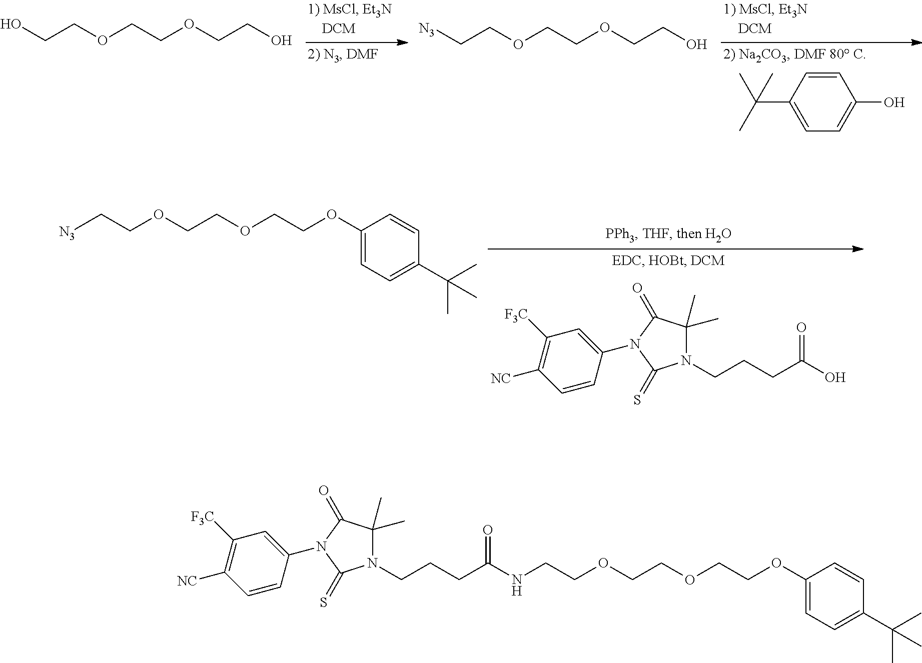 [SMILES: OCCOCCOCCO ; [N-]=[N+]=NCCOCCOCCO ; CC(C)(C)c1ccc(O)cc1 ; CC(C)(C)c1ccc(OCCOCCOCCN=[N+]=[N-])cc1 ; CC1(C)C(=O)N(c2ccc(C#N)c(C(F)(F)F)c2)C(=S)N1CCCC(=O)O ; CC(C)(C)c1ccc(OCCOCCOCCNC(=O)CCCN2C(=S)N(c3ccc(C#N)c(C(F)(F)F)c3)C(=O)C2(C)C)cc1]

### 2-(2-(2-Hydroxyethoxy)ethoxy)ethyl methanesulfonate

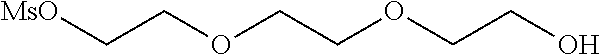 [SMILES: CS(=O)(=O)OCCOCCOCCO]

To a round bottom flask with stirbar and 60 mL distilled DCM was charged 2,2′-(ethane-1,2-diylbis(oxy))diethanol (3.3, 22.0 mmol) and Et₃N (3.0 μL, 22.0 mmol). Upon cooling to 0° C. methanesulfonyl chloride (0.532 mL, 7.0 mmol) was added and the mixture let stir for 18 h at which time the reaction was diluted with 60 mL 1 M Aq. HCl and extracted in to DCM (2×50 mL). The organic layer was washed with water (2×50 mL) and dried with Na₂SO₄ to yield a crude oil. Column chromatography (3:1 to 1:1 Hex:EtOAc) led to the isolation of 400 mg (25% yield) pure product. ¹H NMR (300 MHz, CDCl₃) δ 4.47-4.30 (m, 2H), 3.92-3.53 (m, 10H), 3.08 (s, 3H); ¹³C NMR (126 MHz, CDCl₃) 672.4, 70.7, 70.3, 69.0, 68.9, 61.7, 37.7; LRMS (ESI) 229.2 (M+H)⁺.

### 2-(2-(2-Azidoethoxy)ethoxy)ethanol

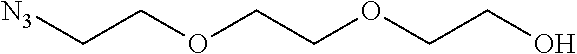 [SMILES: [N-]=[N+]=NCCOCCOCCO]

To a 2 dram vial with stirbar and 10 mL DMF was charged 2-(2-(2-hydroxyethoxyl)ethoxy)ethyl methanesulfonate (450 mg, 2.0 mmol) and sodium azide (1.3 g, 20 mmol). Upon stirring at 80° C. for 18 h the reaction was diluted with 10 mL 1 M HCl and extracted into EtOAc (2×10 mL). The organic layer was washed with water (2×10 mL) and dried with Na₂SO₄, and concentrated down to yield 350 mg (quant. yield) of product as an oil. ¹H NMR (500 MHz, CDCl₃) δ 3.77-3.72 (m, 2H), 3.68 (s, 6H), 3.64-3.60 (m, 2H), 3.43-3.37 (m, 2H); ¹³C NMR (125 MHz, CDCl₃) δ 72.5, 70.6, 70.4, 70.0, 61.7, 50.6; LRMS 198.2 (M+Na)⁺.

### 2-(2-(2-Azidoethoxy)ethoxy)ethyl methanesulfonate

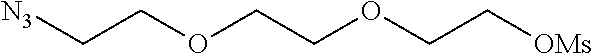 [SMILES: CS(=O)(=O)OCCOCCOCCN=[N+]=[N-]]

To a round bottom flask with stirbar and 30 mL distilled DCM was charged 2-(2-(2-azidoethoxyl)ethoxy)ethanol (350 mg, 2.0 mmol) and Et₃N (1.12 mL, 4.0 mmol). Upon cooling to 0° C. methanesulfonyl chloride (0.46 μL, 6.0 mmol) was added and the mixture let stir for 18 h at which time the reaction was diluted with 30 mL 1M Aq. HCl and extracted into DCM (2×50 mL). The organic layer was washed with water (2×50 mL) and dried with Na₂SO₄ to yield a crude oil. Column chromatography (5:1 to 1:1 Hex:EtOAc) led to the isolation of 185 mg (37% yield) pure product. ¹H NMR (500 MHz, CDCl₃) δ 4.42-4.28 (m, 2H), 3.81-3.71 (m, 2H), 3.71-3.58 (m, 6H), 3.35 (d, J=4.5, 2H), 3.06-3.02 (m, 3H). LRMS (ESI) 276.4 (M+Na)⁺.

### 1-(2-(2-(2-Azidoethoxy)ethoxy)ethoxy)-4-(tert-butyl)benzene

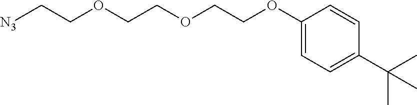 [SMILES: CC(C)(C)c1ccc(OCCOCCOCCN=[N+]=[N-])cc1]

To a 2 dram vial with stir bar was charged the 2-(2-(2-azidoethoxyl) ethoxy)ethyl methanesulfonate (50 mg, 0.2 mmol), Na₂CO₃ (138 mg, 1.0 mmol), phenol (90 mg, 0.6 mmol) and 1 mL DMF. The reaction was left to stir over 18 h at 80° C. upon which time the mixture was diluted with 2 mL H₂O and extracted into EtOAC (3×3 mL). The organic layer was washed with brine (2×5 mL) and dried with Na₂SO₄ and concentrated down to yield a crude oil which was purified by preparative TLC (5:1 Hexanes:EtOAc) to yield 48 mg (78% yield) of product as an oil. ¹H NMR (500 MHz, CDCl₃) δ 7.29 (d, J=8.1, 2H), 6.85 (d, J=8.8, 2H), 4.16-4.01 (m, 2H), 3.94-3.80 (m, 2H), 3.78-3.64 (m, 6H), 3.38 (t, J=5.0, 2H), 1.29 (s, 9H). LRMS (ESI) 329.5 (M+Na)⁺.

### 2-(2-(2-(4-(tert-Butyl)phenoxy)ethoxy)ethoxy)ethanamine

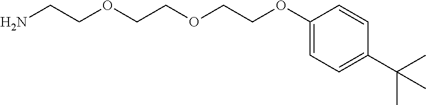 [SMILES: CC(C)(C)c1ccc(OCCOCCOCCN)cc1]

To a 2 dram vial with stirbar and 2 mL THF was charged 1-(2-(2-(2-azidoethoxyl)ethoxy)ethoxy)-4-(tert-butyl)benzene (47 mg, 0.15 mmol) and triphenylphosphine (52 mg, 0.2 mmol). 20 μL of H₂O was added after 2 h and the mixture let stir for 16 h at rt. The solvents were removed via rotovap to yield a crude oil which was purified by column chromatography (DCM to 5:1 DCM:MeOH (0.5 N NH₃) to yield 30 mg (71% yield). ¹H NMR (500 MHz, CDCl₃) δ 7.32-7.16 (m, 2H), 6.81 (d, J=7.4, 2H), 4.08 (d, J=3.8, 2H), 3.81 (d, J=3.8, 2H), 3.68 (s, 2H), 3.61 (d, J=3.3, 2H), 3.48 (s, 2H), 2.83 (s, 2H), 1.33-1.16 (m, 9H); ¹³C NMR (125 MHz, CDCl₃) δ 156.4, 143.4, 126.1, 114.0, 70.7, 70.3, 70.0, 67.4, 67.4, 67.3, 34.0, 31.5; LRMS (ESI) 282.4 (M+H)⁺.

### SARD 3-190

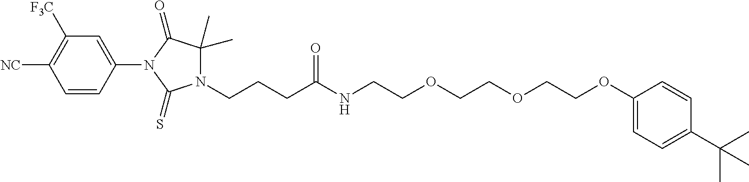 [SMILES: CC(C)(C)c1ccc(OCCOCCOCCNC(=O)CCCN2C(=S)N(c3ccc(C#N)c(C(F)(F)F)c3)C(=O)C2(C)C)cc1]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (35.0 mg, 0.087 mmol), EDC (22 mg, 0.12 mmol), HOBt (18 mg, 0.12 mmol), and 1.0 mL DCM. After 15 minutes of stirring 2-(2-(2-(4-(tert-butyl)phenoxy)ethoxy)ethoxy)ethanamine (19 mg, 0.075 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by preparative TLC (19:1 DCM:MeOH) to yield 33 mg (57% yield) of pure product as an amber oil. ¹H NMR (500 MHz, CDCl₃) δ 7.94 (d, J=8.3, 1H), 7.90 (d, J=2.0, 1H), 7.76 (dd, J=2.0, 8.2, 1H), 7.32-7.28 (m, 2H), 6.91-6.80 (m, 2H), 4.13 (dd, J=4.0, 5.4, 2H), 3.86 (dd, J=4.0, 5.4, 2H), 3.77-3.69 (m, 4H), 3.69-3.62 (m, 2H), 3.63-3.55 (m, 2H), 3.47 (dd, J=5.3, 10.3, 2H), 2.29 (t, J=6.7, 2H), 2.11 (dt, J=6.9, 16.3, 2H), 1.60 (s, 6H), 1.29 (s, 9H); ¹³C NMR (126 MHz, CDCl₃) δ 178.4, 175.3, 171.7, 156.4, 143.9, 137.2, 135.0, 133.4 (q, J=30.4), 132.0, 127.0 (q, J=4.5), 126.3 121.9 (q, J=277.3), 114.8, 114.2, 110.0, 70.7, 70.3, 69.8, 69.8, 67.6, 65.2, 43.6, 39.3, 34.0, 32.9, 31.5, 23.6, 23.0; LRMS (ESI) 660.3 (M+H)⁺.

### Example 22

### 4-(3-(4-Cyano-3-(trifluoromethyl)phenyl)-5,5-dimethyl-4-oxo-2-thioxoimidazolidin-1-yl)-N-(2-(2-(2-(quinolin-8-yloxy)ethoxy)ethoxy)ethyl) butanamide (SARD 3-191)

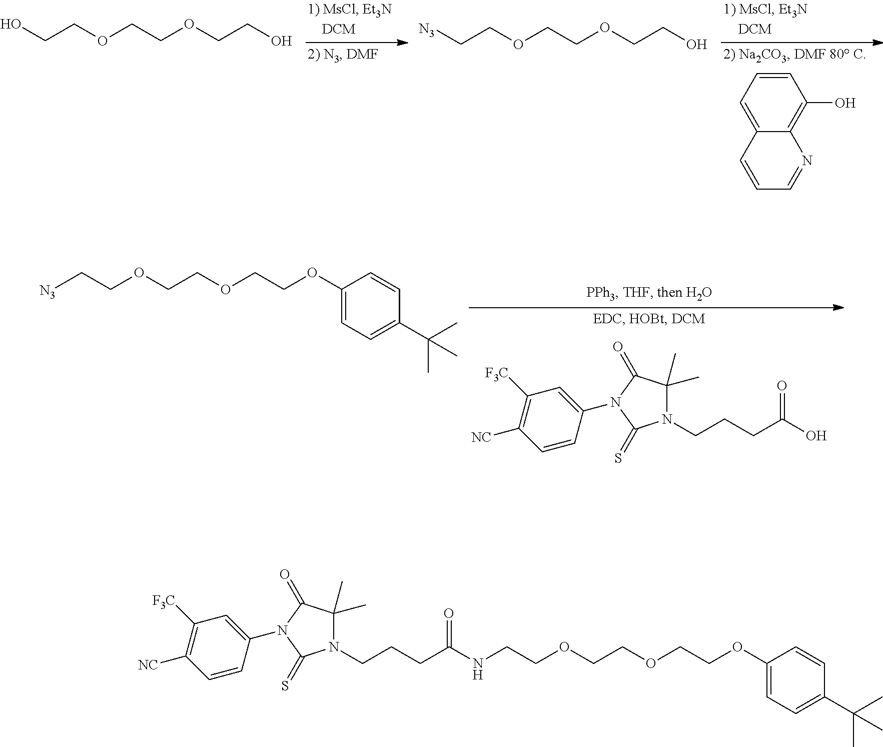 [SMILES: OCCOCCOCCO ; [N-]=[N+]=NCCOCCOCCO ; Oc1cccc2cccnc12 ; CC(C)(C)c1ccc(OCCOCCOCCN=[N+]=[N-])cc1 ; CC1(C)C(=O)N(c2ccc(C#N)c(C(F)(F)F)c2)C(=S)N1CCCC(=O)O ; CC(C)(C)c1ccc(OCCOCCOCCNC(=O)CCCN2C(=S)N(c3ccc(C#N)c(C(F)(F)F)c3)C(=O)C2(C)C)cc1]

### 8-(2-(2-(2-Azidoethoxy)ethoxy)ethoxy)quinoline

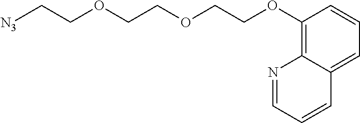 [SMILES: [N-]=[N+]=NCCOCCOCCOc1cccc2cccnc12]

To a 2 dram vial with stir bar was charged 2-(2-(2-azidoethoxyl)ethoxy)ethyl methanesulfonate (50 mg, 0.2 mmol), Na₂CO₃ (138 mg, 1.0 mmol), hydroxyquinoline (87 mg, 0.6 mmol) and 1 mL DMF. The reaction was left to stir over 18 h at 80° C. upon which time the mixture was diluted with 2 mL H₂O and extracted into EtOAC (3×3 mL). The organic layer was washed with brine (2×5 mL) and dried with Na₂SO₄ and concentrated down to yield a 40 mg (66% yield) of the product as an amber oil. ¹H NMR (500 MHz, CDCl₃) δ 8.90 (s, 1H), 8.09 (d, J=8.6, 1H), 7.39 (dt, J=8.1, 21.6, 3H), 7.09 (d, J=7.5, 1H), 4.40 (t, J=5.2, 2H), 4.04 (t, J=4.9, 2H), 3.75 (m, 2H), 3.70-3.59 (m, 4H), 3.33 (s, 2H); LRMS (ESI) 303.2 (M+H)⁺.

### 2-(2-(2-(Quinolin-8-yloxy)ethoxy)ethoxy)ethanamine

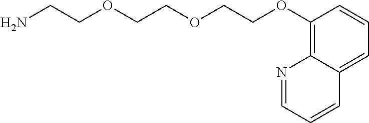 [SMILES: NCCOCCOCCOc1cccc2cccnc12]

To a 2 dram vial with stirbar and 2 mL THF was charged 8-(2-(2-(2-azidoethoxyl)ethoxy)ethoxy)quinoline (40 mg, 0.13 mmol) and triphenylphosphine (43 mg, 0.18 mmol). 200 μL of H₂O was added after 2 h and the mixture let stir for 16 h at rt. The solvents were removed via rotovap to yield a crude oil which was purified by column chromatography (DCM to 4:1 DCM:MeOH (0.5 N NH₃) to yield 36 mg (95% yield). ¹H NMR (500 MHz, CDCl₃) δ 8.90 (dd, J=1.7, 4.2, 1H), 8.09 (dd, J=1.7, 8.3, 1H), 7.39 (ddt, J=4.7, 8.3, 9.6, 3H), 7.08 (dd, J=1.3, 7.6, 1H), 4.45-4.33 (m, 2H), 4.08-4.02 (m, 2H), 3.79-3.74 (m, 2H), 3.65-3.60 (m, 2H), 3.52-3.43 (m, 2H), 2.83 (dd, J=7.6, 12.9, 2H); ¹³C NMR (125 MHz, CDCl₃) δ 154.6, 149.2, 140.4, 135.8, 129.5, 126.6, 121.5, 119.9, 109.3, 73.4, 70.8, 70.3, 69.5, 68.2, 41.8.

### SARD 3-191

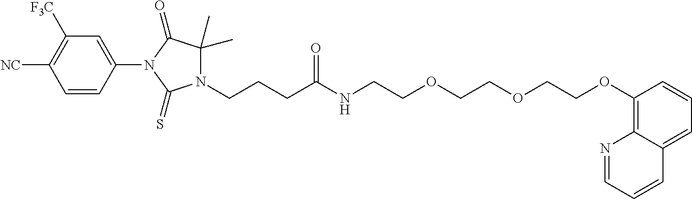 [SMILES: CC1(C)C(=O)N(c2ccc(C#N)c(C(F)(F)F)c2)C(=S)N1CCCC(=O)NCCOCCOCCOc1cccc2cccnc12]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (35.0 mg, 0.087 mmol), EDC (26 mg, 0.14 mmol), HOBt (22 mg, 0.14 mmol), and 1.0 mL DCM. After 15 minutes of stirring 2-(2-(2-(quinolin-8-yloxy)ethoxy)ethoxy) ethanamine (35 mg, 0.126 mmol) was added and the mixture left stir for 16 h upon which the mixture was diluted with 1 mL DCM and washed with saturated NaHCO₃ (2×1 mL). The organic layer was dried with Na₂SO₄ and concentrated down to yield a crude oil which was purified by preparative TLC (19:1 DCM:MeOH) to yield XX mg (57% yield) of pure product as an amber oil. ¹H NMR (500 MHz, CDCl₃) δ 8.83 (dd, J=1.7, 4.3, 1H), 8.11 (dd, J=1.7, 8.3, 1H), 7.86 (d, J=8.3, 1H), 7.82 (d, J=2.0, 2H), 7.68 (dd, J=2.0, 8.3, 1H), 7.43-7.33 (m, 3H), 7.00 (dd, J=1.2, 7.6, 1H), 4.31 (dd, J=3.6, 5.4, 2H), 4.01-3.92 (m, 2H), 3.71 (ddd, J=3.6, 6.6, 12.8, 2H), 3.63-3.57 (m, 4H), 3.54-3.48 (m, 2H), 3.42 (dd, J=5.2, 9.9, 2H), 2.23 (t, J=6.6, 2H), 2.01 (m, 2H), 1.50 (s, 6H); ¹³C NMR (126 MHz, CDCl₃) δ 178.3, 175.3, 172.0, 154.3, 148.8, 139.8, 137.2, 136.5, 135.0, 133.4 (q, J=33.4), 132.0, 129.6, 127.0 (q, J=4.8), 126.8, 121.9 (q, J=275.5), 121.8, 120.1, 114.8, 110.0, 109.0, 70.8, 70.1, 69.9 69.5, 68.03, 65.2, 43.7, 39.4, 32.6, 23.5, 23.0; LRMS (ESI) 657.1 (M+H)⁺.

### Example 23

### 4-(3-(4-Cyano-3-(trifluoromethyl)phenyl)-5,5-dimethyl-4-oxo-2-thioxoimidazolidin-1-yl)-N-(2-(2-(2-(3,5-dimethylphenoxy)ethoxy)ethoxy)ethyl) butanamide

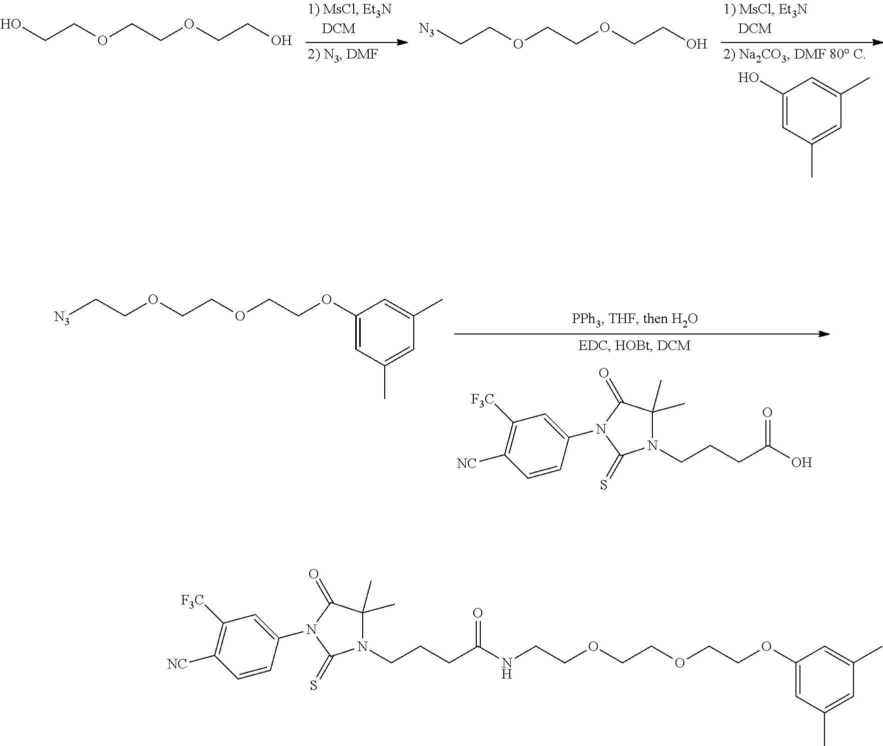

### 1-(2-(2-(2-Azidoethoxy)ethoxy)ethoxy)-3,5-dimethylbenzene

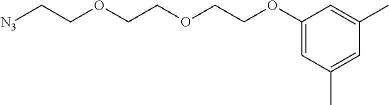 [SMILES: Cc1cc(C)cc(OCCOCCOCCN=[N+]=[N-])c1]

To a 2 dram vial with stir bar was charged 2-(2-(2-azidoethoxyl)ethoxy) ethyl methanesulfonate (50 mg, 0.2 mmol), Na₂CO₃ (138 mg, 1.0 mmol), 3,5-dimethyl phenol (50 mg, 0.4 mmol) and 1 mL DMF. The reaction was left to stir over 18 h at 80° C. upon which time the mixture was diluted with 2 mL H₂O and extracted into EtOAc (3×3 mL). The organic layer was washed with brine (2×5 mL) and dried with Na₂SO₄ and concentrated down to yield a crude oil which was purified by preparative TLC (1:1 Hexanes:EtOAc) to yield 58 mg (67% yield) of product as an oil. ¹H NMR (500 MHz, CDCl₃) δ 6.52 (s, 1H), 6.47 (s, 2H), 4.06-3.94 (m, 2H), 3.77 (dd, J=4.3, 5.5, 2H), 3.66 (dt, J=1.4, 4.5, 2H), 3.64-3.55 (m, 4H), 3.34-3.26 (m, 2H), 2.22-2.17 (s, 6H); ¹³C NMR (126 MHz, CDCl₃) δ 159.20, 139.6, 123.1, 112.8, 71.3, 71.2, 70.5, 70.3, 67.7, 51.1, 21.8; LRMS (ESI) 280.7 (M+H)⁺.

### 2-(2-(2-(3,5-Dimethylphenoxy)ethoxy)ethoxy)ethanamine

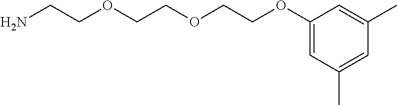 [SMILES: Cc1cc(C)cc(OCCOCCOCCN)c1]

To a 2 dram vial with stirbar and 2 mL THF was charged 1-(2-(2-(2-azidoethoxyl)ethoxy)ethoxy)-3,5-dimethylbenzene (38 mg, 0.14 mmol) and triphenylphosphine (46 mg, 0.17 mmol). 200 μL of H₂O was added after 2 h and the mixture let stir for 16 h at rt. The solvents were removed via rotovap to yield a crude oil which was purified by column chromatography (DCM to 5:1 DCM:MeOH (0.5 N NH₃) to yield 30 mg (87% yield). ¹H NMR (500 MHz, CDCl₃) δ 6.59 (s, 1H), 6.54 (s, 2H), 4.13-4.06 (m, 2H), 3.87-3.81 (m, 2H), 3.75-3.68 (m, 4H), 3.67-3.62 (m, 2H), 3.52 (t, J=5.2, 2H), 2.86 (s, 2H), 2.27 (s, 6H); ¹³C NMR (126 MHz, CDCl₃) δ 158.82, 139.1, 122.5, 112.5, 73.3, 70.8, 70.3, 69.8, 67.3, 41.8, 21.4; LRMS (ESI) 253.1 (M+H)⁺.

### SARD 3-(209)

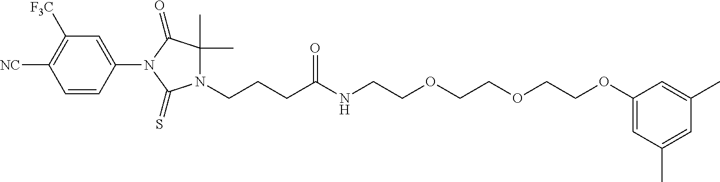 [SMILES: Cc1cc(C)cc(OCCOCCOCCNC(=O)CCCN2C(=S)N(c3ccc(C#N)c(C(F)(F)F)c3)C(=O)C2(C)C)c1]

To a 1 dram vial with stirbar was charged 4-(3-(4-cyano-3-(trifluoromethyl)phenyl)-5,5-dimethyl-4-oxo-2-thioxoimidazolidin-1-yl)butanoic acid (35.0 mg, 0.087 mmol), EDC (19 mg, 0.1 mmol), HOBt (16 mg, 0.1 mmol), and 1.0 mL DCM. After 15 minutes of stirring 2-(2-(2-(3,5-dimethylphenoxy)ethoxy)ethoxy) ethanamine (30 mg, 0.12 mmol) followed by DIPEA (24 μL, 0.14 mmol) was added and the mixture left stir for 16 h upon which the mixture was diluted with 1 mL DCM and washed with 10% aq. citric acid (2×1 mL), and saturated Na₂CO₃ (2×1 mL). The organic layer was dried with Na₂SO₄ and concentrated down to yield a crude oil which was purified by preparative TLC (9:1 DCM:MeOH) to yield 38 mg (65% yield) of pure product as an amber oil. ¹H NMR (500 MHz, CDCl₃) δ 7.94 (d, J=8.2, 1H), 7.90 (d, J=2.0, 1H), 7.76 (dd, J=2.1, 8.3, 1H), 6.61 (s, 1H), 6.54 (s, 2H), 6.29 (s, 1H), 4.17-4.06 (m, 2H), 3.88-3.82 (m, 2H), 3.75-3.70 (m, 4H), 3.68-3.62 (m, 2H), 3.63-3.55 (m, 2H), 3.47 (dd, J=5.2, 10.3, 2H), 2.30-2.25 (m, 9H); ¹³C NMR (126 MHz, CDCl₃) δ 178.7, 175.8, 172.1, 159.4, 139.7, 137.5, 135.5, 133.8 (q, J=32.6), 132.5, 127.4 (q, J=5.0), 123.3, 122.3 (q, J=275.3), 115.3, 112.8, 110.3, 71.1, 70.7, 70.3, 70.2, 67.7, 65.7, 44.0, 39.7, 33.2, 23.9, 23.5, 22.0; LRMS (ESI) 635.2 (M+H)⁺.

### Example 24

### N-(14-(Adamantan-1-yloxy)-3,6,9,12-tetraoxatetradecyl)-4-(3-(4-cyano-3-(trifluoromethyl)phenyl)-5,5-dimethyl-4-oxo-2-thioxoimidazolidin-1-yl)butanamide (SARD 7-156)

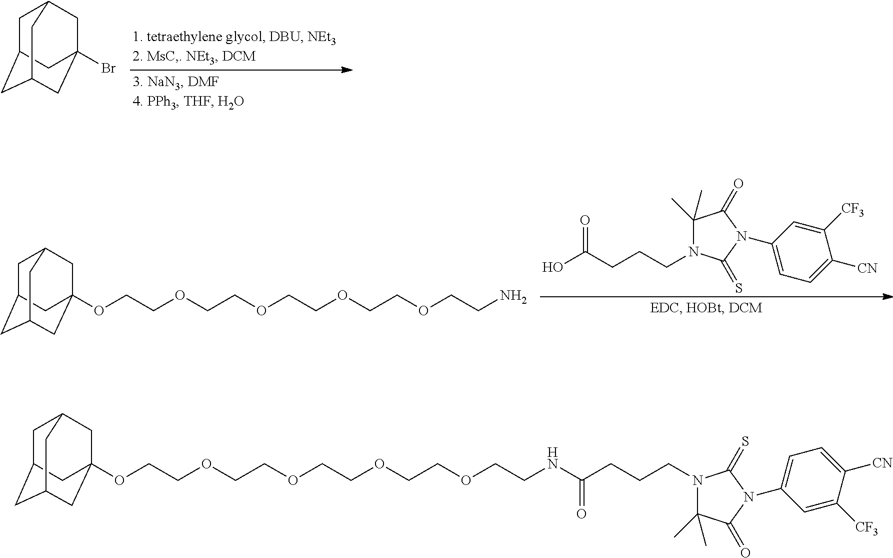 [SMILES: BrC12CC3CC(CC(C3)C1)C2 ; NCCOCCOCCOCCOCCOC12CC3CC(CC(C3)C1)C2 ; CC1(C)C(=O)N(c2ccc(C#N)c(C(F)(F)F)c2)C(=S)N1CCCC(=O)O ; CC1(C)C(=O)N(c2ccc(C#N)c(C(F)(F)F)c2)C(=S)N1CCCC(=O)NCCOCCOCCOCCOCCOC12CC3CC(CC(C3)C1)C2]

### 14-(Adamantan-1-yloxy)-3,6,9,12-tetraoxatetradecan-1-ol

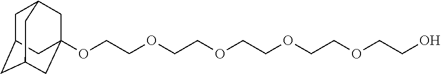 [SMILES: OCCOCCOCCOCCOCCOC12CC3CC(CC(C3)C1)C2]

1-Bromoadamantane (0.305 g, 1.42 mmol, 1 eq) was dissolved in triethylamine (0.6 mL, 4.3 mmol, 3 eq). DBU (11 μL, 0.07 mmol, 5 mol %) and pentaethylene glycol (1.5 mL, 7.1 mmol, 5 eq) were added and the mixture was heated to 110° C. for 18 hours. The mixture was cooled to room temperature, diluted with 1M Hcl and extracted with DCM. The organic layer was dried over sodium sulfate, filtered and condensed. Purification by column chromatography (1 to 5% MeOH/DCM) gave a light yellow oil (0.37 g, 0.99 mmol, 70%). ¹H NMR (400 MHz, CDCl₃) δ 3.72 (dd, J=9.2, 5.7 Hz, 2H), 3.70-3.64 (m, 12H), 3.63-3.56 (m, 6H), 2.61 (t, J=6.2 Hz, 1H), 2.13 (s, 3H), 1.74 (d, J=2.8 Hz, 6H), 1.61 (q, J=12.3 Hz, 6H). MS (ESI) 373.5 (M+H)⁺.

### 14-(Adamantan-1-yloxy)-3,6,9,12-tetraoxatetradecyl methanesulfonate

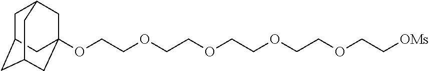 [SMILES: CS(=O)(=O)OCCOCCOCCOCCOCCOC12CC3CC(CC(C3)C1)C2]

14-(adamantan-1-yloxy)-3,6,9,12-tetraoxatetradecan-1-ol (0.1517 g, 0.407 mmol, 1 eq) was dissolved in DCM (1.6 mL) at room temperature. Triethylamine (0.17 mL, 1.22 mmol, 3 eq) and methanesulfonyl chloride (47.3 μL, 0.611 mmol, 1.5 eq) were added and the solution was stirred for 14 hours. The mixture was diluted with 10% citric acid, extracted thrice with DCM, dried with sodium sulfate, filtered and condensed. Purification by column chromatography (50 to 100% EtOAc/hexanes) gave a colorless oil. (0.14 g, 0.311 mmol, 76%). ¹H NMR (400 MHz, CDCl₃) δ 4.38-4.30 (m, 2H), 3.76-3.69 (m, 2H), 3.65-3.58 (m, 12H), 3.57-3.50 (m, 4H), 3.05 (s, 3H), 2.10 (s, 3H), 1.70 (d, J=2.7 Hz, 6H), 1.57 (q, J=12.2 Hz, 6H). MS (ESI) 451.0 (M+H)⁺.

### 1-((Adamantan-1-yloxy)-14-azido-3,6,9,12-tetraoxatetradecane

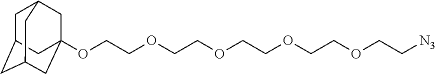 [SMILES: [N-]=[N+]=NCCOCCOCCOCCOCCOC12CC3CC(CC(C3)C1)C2]

14-(adamantan-1-yloxy)-3,6,9,12-tetraoxatetradecyl methanesulfonate (0.14 g, 0.311 mmol, 1 eq) was dissolved in DMF (1 mL). Sodium azide (0.060 g, 0.933 mmol, 3 eq) was added and the mixture was heated to 100° C. for 12 hours. The mixture was then cooled to room temperature, diluted with water and extracted thrice with EtOAc. The organic layer was then dried over sodium sulfate, filtered and condensed to give a yellow oil (98.3 mg, 0.247 mmol, 79%) that was deemed sufficiently pure. ¹H NMR (501 MHz, CDCl₃) δ 3.70-3.62 (m, 14H), 3.61-3.55 (m, 4H), 3.43-3.35 (m, 2H), 2.14 (s, 3H), 1.74 (d, J=2.7 Hz, 6H), 1.61 (q, J=12.2 Hz, 6H). ¹³C NMR (126 MHz, CDCl₃) δ 72.39, 71.43, 70.86, 70.84, 70.80, 70.76, 70.76, 70.19, 59.41, 50.85, 41.64, 36.62, 30.66. MS (ESI) 369.6 (M-N₂), 419.1 (M+Na)⁺.

### 14-(Adamantan-1-yloxy)-3,6,9,12-tetraoxatetradecan-1-amine

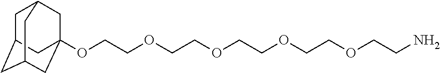 [SMILES: NCCOCCOCCOCCOCCOC12CC3CC(CC(C3)C1)C2]

1-(adamantan-1-yloxy)-14-azido-3,6,9,12-tetraoxatetradecane (98.3 mg, 0.247 mmol, 1 eq) was dissolved in THF (0.62 mL) at room temperature. Triphenylphosphine (110 mg, 0.42 mmol, 1.7 eq) was added and the solution was stirred for 3 hours, upon which water (0.31 mL) was added. After 13 hours, the mixture was diluted with EtOAc, extracted twice with 1M HCl, basified with 3M NaOH, and extracted 5 times with chloroform. The combined organic layer was then dried over sodium sulfate, filtered, and condensed. Purification by column chromatography (1 to 20% 0.5N methanolic ammonia/DCM) gave a colorless oil (66.7 mg, 0.18 mmol, 73%). ¹H NMR (500 MHz, CDCl₃) δ 3.69-3.58 (m, 12H), 3.58-3.52 (m, 4H), 3.50 (t, J=5.1 Hz, 2H), 2.85 (br s, 2H), 2.11 (s, 3H), 1.98 (br s, 2H), 1.71 (d, J=2.5 Hz, 6H), 1.58 (q, J=12.2 Hz, 6H). ¹³C NMR (126 MHz, CDCl₃) δ 73.27, 72.37, 71.36, 70.65, 70.61, 70.36, 59.31, 41.55, 36.53, 30.57. MS (ESI) 372.7 (M+H)⁺.

### DB-7-156

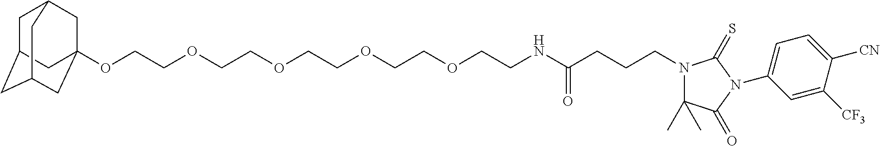 [SMILES: CC1(C)C(=O)N(c2ccc(C#N)c(C(F)(F)F)c2)C(=S)N1CCCC(=O)NCCOCCOCCOCCOCCOC12CC3CC(CC(C3)C1)C2]

4-(3-(4-cyano-3-(trifluoromethyl)phenyl)-5,5-dimethyl-4-oxo-2-thioxoimidazolidin-1-yl)butanoic acid (20 mg, 0.050 mmol, 1 eq), EDC (10.5 mg, 0.055 mmol, 1.1 eq) and HOBt (7.2 mg, 0.055 mmol, 1.1 eq) were dissolved in DCM (0.1 mL) at room temperature. DIPEA (9 μL, 0.050 mmol, 1 eq) was added and the solution was stirred for 25 minutes, upon which 14-((adamantan-1-yloxy)-3,6,9,12-tetraoxatetradecan-1-amine (0.241 mL, 0.065 mmol, 1.3 eq) was added as 100 mg/mL solution in DCM. The solution was stirred for 20 hours, then diluted with half saturated NaCl. The mixture was extracted thrice with EtOAc, then dried over sodium sulfate, filtered and condensed. Purification by preparative TLC (5% MeOH/DCM, rf=0.4) gave a colorless oil (29.3 mg, 0.0389 mmol, 78%). ¹H NMR (500 MHz, CDCl₃) δ 7.94 (d, J=8.3 Hz, 1H), 7.89 (d, J=1.7 Hz, 1H), 7.77 (dd, J=8.3, 1.9 Hz, 1H), 6.90 (s, 1H), 3.81-3.73 (m, 2H), 3.68-3.61 (m, 12H), 3.57 (dt, J=8.6, 4.2 Hz, 6H), 3.45 (dd, J=10.1, 5.2 Hz, 2H), 2.33 (t, J=6.7 Hz, 2H), 2.12 (s, 3H), 1.72 (d, J=2.5 Hz, 6H), 1.69-1.41 (m, 14H). ¹³C NMR (126 MHz, CDCl₃) δ 178.42, 175.51, 172.01, 137.30, 135.21, 133.54 (q, J=33.5 Hz), 132.20, 127.16 (q, J=4.7 Hz), 122.01 (q, J=274.3 Hz), 114.97, 110.03, 72.47, 71.40, 70.66, 70.63, 70.60, 70.28, 69.92, 65.45, 59.34, 43.79, 41.59, 39.42, 36.55, 32.77, 30.60, 23.64, 23.18. MS (ESI) 753.7 (M+H)⁺.

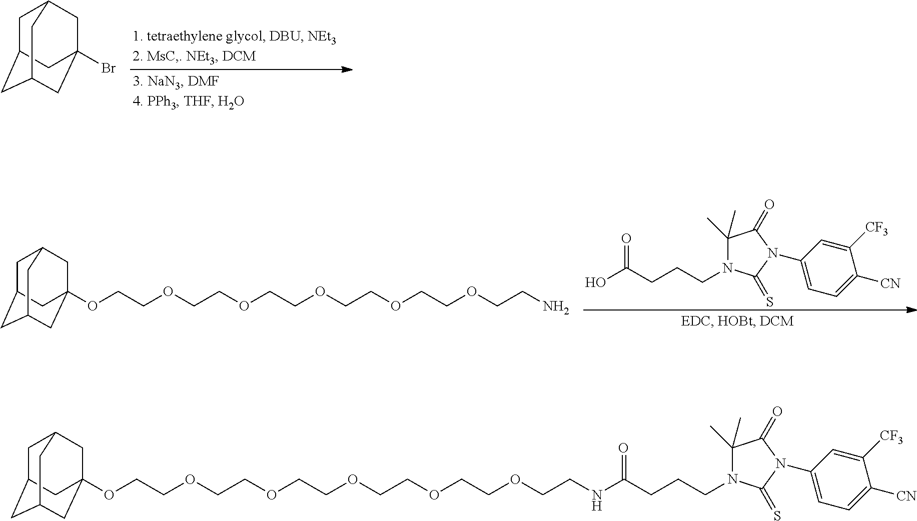 [SMILES: BrC12CC3CC(CC(C3)C1)C2 ; NCCOCCOCCOCCOCCOCCOC12CC3CC(CC(C3)C1)C2 ; CC1(C)C(=O)N(c2ccc(C#N)c(C(F)(F)F)c2)C(=S)N1CCCC(=O)O ; CC1(C)C(=O)N(c2ccc(C#N)c(C(F)(F)F)c2)C(=S)N1CCCC(=O)NCCOCCOCCOCCOCCOCCOC12CC3CC(CC(C3)C1)C2]

### Example 25

### N-(17-(Adamantan-1-yloxy)-3,6,9,12,15-pentaoxaheptadecyl)-4-(3-(4-cyano-3-(trifluoromethyl)phenyl)-5,5-dimethyl-4-oxo-2-thioxoimidazolidin-1-yl)butanamide (SARD 7-150)

### 17-(Adamantan-1-yloxy)-3,6,9,12,15-pentaoxaheptadecan-1-ol

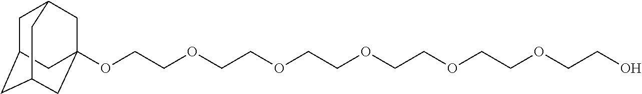 [SMILES: OCCOCCOCCOCCOCCOCCOC12CC3CC(CC(C3)C1)C2]

1-Bromoadamantane (0.305 g, 1.42 mmol, 1 eq) was dissolved in triethylamine (0.6 mL, 4.26 mmol, 3 eq). DBU (11 μL, 0.07 mmol, 5 mol %) and hexaethylene glycol (1.8 mL, 7.1 mmol, 5 eq) were added and the mixture was heated to 110° C. for 20 hours. The mixture was diluted with 1M HCl and extracted thrice with DCM. The organic layer was dried with sodium sulfate, filtered and condensed. Purification by column chromatography (1 to 10% MeOH/DCM) gave a colorless oil (0.39 g, 0.936 mmol, 66%). ¹H NMR (501 MHz, cdcl₃) δ 3.58-3.56 (m, 2H), 3.51 (dd, J=3.6, 2.2 Hz, 16H), 3.46-3.42 (m, 6H), 1.99 (s, 3H), 1.60 (d, J=2.6 Hz, 6H), 1.47 (q, J=12.2 Hz, 6H). MS (ESI) 416.9 (M+H)⁺.

### 17-(Adamantan-1-yloxy)-3,6,9,12,15-pentaoxaheptadecyl methanesulfonate

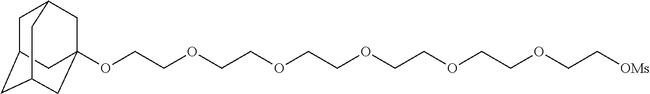 [SMILES: CS(=O)(=O)OCCOCCOCCOCCOCCOCCOC12CC3CC(CC(C3)C1)C2]

17-(adamantan-1-yloxy)-3,6,9,12,15-pentaoxaheptadecan-1-ol (1.49 g, 3.58 mmol, 1 eq) was dissolved in DCM (15 mL) at room temperature. Triethylamine (1.46 mL, 10.7 mmol, 3 eq) and methanesulfonyl chloride (0.42 mL, 5.4 mmol, 1.5 eq) were added and the solution was stirred for 17 hours. The mixture was then diluted with 10% citric acid and extracted twice with DCM. The combined organic layers were dried over sodium sulfate, filtered and condensed. Purification by column chromatography (50 to 100% EtOAc/hexanes) gave a colorless oil (0.73 g, 1.47 mmol, 41%). MS (ESI) 495.4 (M+H), 517.3 (M+Na).

### 1-(Adamantan-1-yloxy)-17-azido-3,6,9,12,15-pentaoxaheptadecane

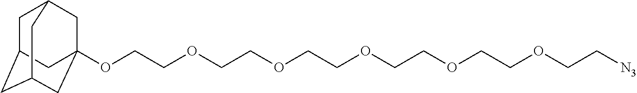 [SMILES: [N-]=[N+]=NCCOCCOCCOCCOCCOCCOC12CC3CC(CC(C3)C1)C2]

17-(adamantan-1-yloxy)-3,6,9,12,15-pentaoxaheptadecyl methanesulfonate (0.73 g, 1.47 mmol, 1 eq) was dissolve din DMF (5.9 mL). Sodium azide (0.24 g, 3.7 mmol, 2.5 eq) was added and the mixture was heated to 100° C. for 11 hours. The mixture was then diluted with water and extracted thrice with EtOAc. The combined organic layer was dried over sodium sulfate, filtered and condensed. Purification by column chromatography (1 to 5% MeOH/DCM) gave a light yellow oil (0.46 g, 1.04 mmol, 71%). ¹H NMR (501 MHz, CDCll₃) δ 3.53 (dd, J=5.7, 2.7 Hz, 16H), 3.48-3.41 (m, 4H), 3.26 (t, J=5.0 Hz, 4H), 2.01 (s, 3H), 1.61 (d, J=2.1 Hz, 5H), 1.49 (q, J=12.2 Hz, 5H). ¹³C NMR (126 MHz, cdcl₃) δ 71.89, 70.98, 70.40, 70.39, 70.35, 70.31, 69.77, 59.00, 50.40, 41.22, 36.21, 30.23. MS (ESI) 414.9 (M-N₂), 463.4 (M+Na)⁺.

### 17-(Adamantan-1-yloxy)-3,6,9,12,15-pentaoxaheptadecan-1-amine

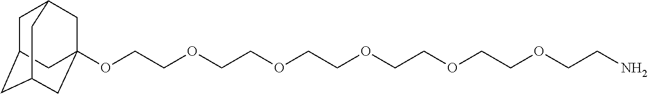 [SMILES: NCCOCCOCCOCCOCCOCCOC12CC3CC(CC(C3)C1)C2]

1-(adamantan-1-yloxy)-17-azido-3,6,9,12,15-pentaoxaheptadecane (0.193 g, 0.437 mmol, 1 eq) was dissolved in THF (1.1 mL) at room temperature. Triphenylphosphine (0.138 g, 0.524 mmol, 1.2 eq) was added and the solution was stirred for 2.5 hours. Water (24 μL) was then added and the solution was stirred for 15 hours. The mixture was then diluted with EtOAc, extracted twice with 1M HCl, basified with 3M NaOH and extracted four times with chloroform. The combined organic layer was dried over sodium sulfate, filtered and condensed. Purification by column chromatography (1 to 20% 0.5N methanolic ammonia/DCM) gave a light yellow oil (97.6 mg, 0.235 mmol, 54%). ¹H NMR (400 MHz, CDCl₃) δ 3.62 (dd, J=3.1, 2.4 Hz, 16H), 3.55 (dd, J=5.0, 3.1 Hz, 4H), 3.51 (d, J=5.3 Hz, 2H), 2.86 (s, 2H), 2.42 (s, 2H), 2.10 (s, 3H), 1.70 (d, J=2.5 Hz, 6H), 1.57 (q, J=12.2 Hz, 6H). ¹³C NMR (126 MHz, cdcl₃) δ 72.32, 71.33, 70.73, 70.66, 70.63, 70.59, 70.54, 70.34, 70.31, 70.07, 59.29, 50.74, 41.54, 36.52, 30.56. MS (ESI) 415.8 (M+H)⁺.

### SARD 7-150

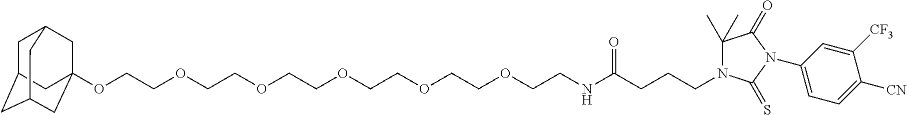 [SMILES: CC1(C)C(=O)N(c2ccc(C#N)c(C(F)(F)F)c2)C(=S)N1CCCC(=O)NCCOCCOCCOCCOCCOCCOC12CC3CC(CC(C3)C1)C2]

4-(3-(4-cyano-3-(trifluoromethyl)phenyl)-5,5-dimethyl-4-oxo-2-thioxoimidazolidin-1-yl)butanoic acid (20 mg, 0.050 mmol, 1 eq), EDC (10.5 mg, 0.055 mmol, 1.1 eq) and HOBt (7.2 mg, 0.055 mmol, 1.1 eq) were dissolved in DCM (0.1 mL) at room temperature. DIPEA (9 μL, 0.050 mmol, 1 eq) was added and the solution was stirred for 20 minutes, upon which 17-(adamantan-1-yloxy)-3,6,9,12,15-pentaoxaheptadecan-1-amine (0.27 mL, 0.065 mmol, 1.3 eq) was added as a 100 mg/mL solution in DCM. The mixture was stirred for 21 hours, then diluted with half saturated sodium chloride and extracted thrice with EtOAc. The combined organic layer was dried over sodium sulfate, filtered and condensed. Purification by column chromatography (1 to 10% MeOH/DCM) followed by preparative thin layer chromatography (5% MeOH/DCM) gave a colorless oil (18.1 mg, 0.0227 mmol, 45%). ¹H NMR (400 MHz, CDCl₃) δ 7.95 (d, J=8.3 Hz, 1H), 7.90 (d, J=1.9 Hz, 1H), 7.77 (dd, J=8.3, 1.9 Hz, 1H), 6.68 (s, 1H), 3.80-3.74 (m, 2H), 3.65 (dd, J=7.0, 2.9 Hz, 16H), 3.57 (dd, J=4.4, 3.2 Hz, 6H), 3.46 (dd, J=9.9, 5.2 Hz, 2H), 2.34 (t, J=6.6 Hz, 2H), 2.12 (s, 3H), 1.73 (d, J=2.7 Hz, 6H), 1.69-1.49 (m, 14H). ¹³C NMR (126 MHz, CDCl₃) δ 178.49, 175.53, 171.96, 137.31, 135.25, 133.62 (q, J=27.5 Hz), 132.22, 127.19 (q, J=4.7 Hz), 122.02 (q, J=283.1 Hz), 114.99, 72.44, 71.45, 70.83, 70.72, 70.68, 70.36, 70.19, 69.94, 65.48, 59.39, 50.84, 43.82, 41.65, 39.47, 36.60, 32.85, 31.06, 30.65, 23.67, 23.23. MS (ESI) 797.6 (M+H)⁺.

### Example 26

### 4-(3-(1-(Adamantan-1-yloxy)-3,6,9,12-tetraoxahexadecan-16-yl)-4,4-dimethyl-5-oxo-2-thioxoimidazolidin-1-yl)-2-(trifluoromethyl)benzonitrile (SARD G4-034)

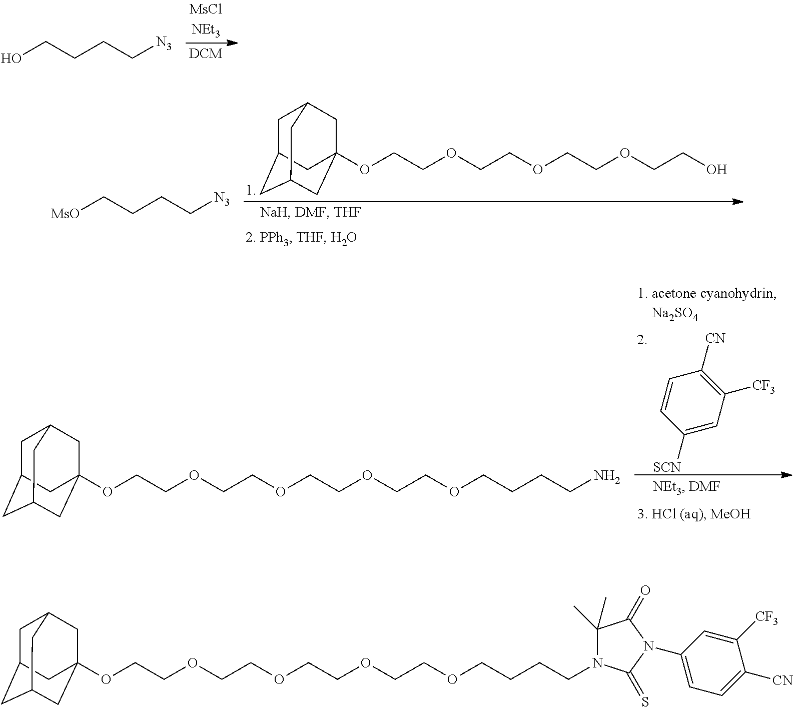

### 4-Azidobutyl methanesulfonate

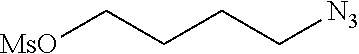 [SMILES: CS(=O)(=O)OCCCCN=[N+]=[N-]]

4-azidobutanol (SynLett, 2009, 20:3275) (1.07 g, 9.3 mmol, 1 eq) was dissolved in DCM (47 mL) at room temperature. After the addition of triethylamine (3.9 mL, 28 mmol, 3 eq), methanesulfonyl chloride (1.1 mL, 14 mmol, 1.5 eq) was added slowly. After 17.5 hours, the mixture was diluted with 10% citric acid and extracted twice with chloroform. The combined organic layer was dried over sodium sulfate, filtered and condensed. Purification by column chromatography (10 to 30% EtOAc/hexanes) gave a colorless oil (1.09 g, 5.64 mol, 60%). ¹H NMR (400 MHz, CDCl₃) δ 4.26 (t, J=6.2 Hz, 2H), 3.36 (t, J=6.6 Hz, 2H), 3.02 (s, 3H), 1.85 (ddd, J=13.5, 7.8, 3.5 Hz, 2H), 1.78-1.68 (m, 2H). MS (ESI) 215.9 (M+Na)⁺.

### 1-(Adamantan-1-yloxy)-16-azido-3,6,9,12-tetraoxahexadecane

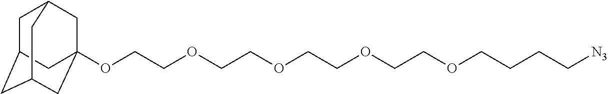 [SMILES: [N-]=[N+]=NCCCCOCCOCCOCCOCCOC12CC3CC(CC(C3)C1)C2]

DMF (6.1 mL) was added to 95% NaH (30 mg, 1.22 mmol, 2 eq) in a dry flask under argon and cooled to 4° C. A solution of 2-(2-(2-(2-(adamantan-1-yloxy)ethoxy)ethoxy)ethoxy)ethanol (0.30 g, 0.913 mmol, 1.5 eq) in dry THF (3 mL) was slowly added via syringe. The mixture was warmed to room temperature for 30 minutes, before being cooled again to 4° C. A solution of 4-azidobutyl methanesulfonate (0.117 g, 0.609 mmol, 1 eq) in dry THF (3 mL) was added via syringe. After 15 minutes, the solution was warmed to room temperature and stirred for 2 hours. The mixture was then quenched with water, diluted with 1M HCl and extracted thrice with chloroform. Purification by column chromatography (25 to 100% EtOAc/hexanes) gave a yellow oil (0.1832 g, 0.430 mmol, 71%). ¹H NMR (500 MHz, CDCl₃) δ 3.69-3.59 (m, 10H), 3.60-3.52 (m, 6H), 3.47 (dd, J=6.9, 5.1 Hz, 2H), 3.28 (t, J=6.5 Hz, 2H), 2.11 (s, 3H), 1.72 (d, J=2.6 Hz, 6H), 1.68-1.53 (m, 10H). ¹³C NMR (126 MHz, CDCl₃) δ 72.33, 71.34, 70.67, 70.24, 59.32, 51.37, 41.54, 36.53, 30.58, 26.86, 25.84. MS (ESI) 398.6 (M-N₂), 3448.0 (M+Na)⁺.

### 1-(Adamantan-1-yloxy)-3,6,9,12-tetraoxahexadecan-16-amine

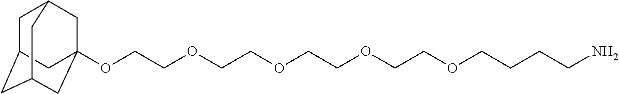 [SMILES: NCCCCOCCOCCOCCOCCOC12CC3CC(CC(C3)C1)C2]

1-(adamantan-1-yloxy)-16-azido-3,6,9,12-tetraoxahexadecane (0.1832 g, 0.430 mmol, 1 eq) was dissolved in THF (1.1 mL) at room temperature. Triphenylphosphine (0.17 g, 0.65 mmol, 1.5 eq) was added and the solution was stirred for 2.5 hours. Water (0.55 mL) was added and the solution was stirred for 19 hours. The mixture was diluted with diethyl ether, extracted twice with 1M HCl, basified with 3M NaOH and extracted five times with chloroform. The combined organic layer was dried over sodium sulfate, filtered and condensed. Purification by column chromatography (1 to 20% 0.5N methanolic ammonia/DCM) to give a colorless oil (0.1183 g, 0.296 mmol, 69%). ¹H NMR (500 MHz, CDCl₃) δ 3.62-3.56 (m, 10H), 3.55-3.49 (m, 6H), 3.42 (t, J=6.4 Hz, 2H), 2.67 (t, J=6.9 Hz, 2H), 2.19 (s, 2H), 2.07 (s, 3H), 1.68 (d, J=2.7 Hz, 6H), 1.62-1.43 (m, 10H). ¹³C NMR (126 MHz, CDCl₃) δ 72.23, 71.25, 71.15, 70.57, 70.54, 70.53, 70.50, 70.05, 59.21, 41.84, 41.46, 36.44, 30.48, 29.98, 27.05. MS (ESI) 400.8 (M+H)⁺.

### 1-(Adamantan-1-yloxy)-18,18-dimethyl-3,6,9,12-tetraoxa-17-azanonadecane-19-nitrile

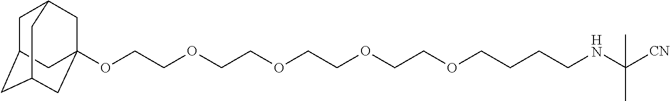 [SMILES: CC(C)(C#N)NCCCCOCCOCCOCCOCCOC12CC3CC(CC(C3)C1)C2]

1-(adamantan-1-yloxy)-3,6,9,12-tetraoxahexadecan-16-amine (0.1183 g, 0.296 mmol) was dissolved in acetone cyanohydrin (0.3 mL). Sodium sulfate (85 mg, 0.6 mmol, 2 eq) was added and the mixture was stirred for 12 hours. The mixture was concentrated under high vacuum and purified by column chromatography (10 to 40% acetone/DCM) to give a light yellow oil (49.6 mg, 0.106 mmol, 36%). ¹H NMR (500 MHz, CDCl₃) δ 3.67-3.61 (m, 10H), 3.59-3.52 (m, 6H), 3.46 (t, J=6.4 Hz, 2H), 2.71 (t, J=7.0 Hz, 2H), 2.12 (s, 3H), 1.72 (d, J=2.2 Hz, 6H), 1.68-1.52 (m, 10H), 1.44 (s, 6H). ¹³C NMR (126 MHz, CDCl₃) δ 123.01, 71.36, 71.07, 70.70, 70.24, 59.34, 44.83, 41.56, 36.55, 30.58, 27.52, 27.42, 26.93.

### SARD G4-034

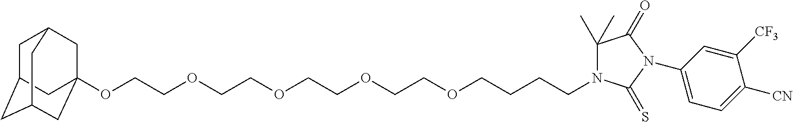 [SMILES: CC1(C)C(=O)N(c2ccc(C#N)c(C(F)(F)F)c2)C(=S)N1CCCCOCCOCCOCCOCCOC12CC3CC(CC(C3)C1)C2]

1-(adamantan-1-yloxy)-18,18-dimethyl-3,6,9,12-tetraoxa-17-azanonadecane-19-nitrile (49.6 mg, 0.106 mmol, 1 eq), 4-isothiocyanato-2-(trifluoromethyl)benzonitrile (29.1 mg, 0.128 mmol, 1.1 eq) and triethylamine (4.5 μL, 0.032 mmol, 0.3 eq) were dissolved in DMF (0.7 mL) and heated to 100° C. for 20.5 hours. Methanol (7.1 mL) and aqueous 1M HCl (1.8 mL) were added and the mixture was heated to 70° C. for 2 hours. The mixture was then diluted with water and extracted four times with DCM. The combined organic layer was dried over sodium sulfate, filtered and condensed. Purification by preparative TLC (100% EtOAc) gave a yellow oil (27.8 mg, 0.040 mmol, 38%). ¹H NMR (500 MHz, CDCl₃) δ 7.94 (d, J=8.3 Hz, 1H), 7.89 (d, J=1.9 Hz, 1H), 7.77 (dd, J=8.2, 2.0 Hz, 1H), 3.72 (dd, J=9.4, 7.1 Hz, 2H), 3.68-3.62 (m, 10H), 3.61-3.55 (m, 6H), 3.53 (t, J=6.2 Hz, 2H), 2.13 (s, 3H), 1.95-1.87 (m, 2H), 1.73 (d, J=2.7 Hz, 6H), 1.68 (dd, J=14.1, 6.7 Hz, 2H), 1.65-1.54 (m, 12H). ¹³C NMR (126 MHz, CDCl₃) δ 178.34, 175.47, 137.27, 135.23, 133.58 (q, J=33.5 Hz), 127.16 (q, J=4.8 Hz), 122.01 (q, J=274.2 Hz), 114.99, 110.07, 72.44, 71.41, 70.74, 70.72, 70.70, 70.69, 70.36, 65.26, 63.54, 59.36, 44.27, 41.59, 36.57, 30.62, 27.14, 25.31, 23.31. MS (ESI) 696.5 (M+H)⁺.

### Example 27

### 4-(3-(4-Cyano-3-(trifluoromethyl)phenyl)-5,5-dimethyl-4-oxo-2-thioxoimidazolidin-1-yl)-N-(2-(2-(2-((4-methoxybenzyl)oxy)ethoxy)ethoxy)ethyl) butanamide (SARD 7-216)

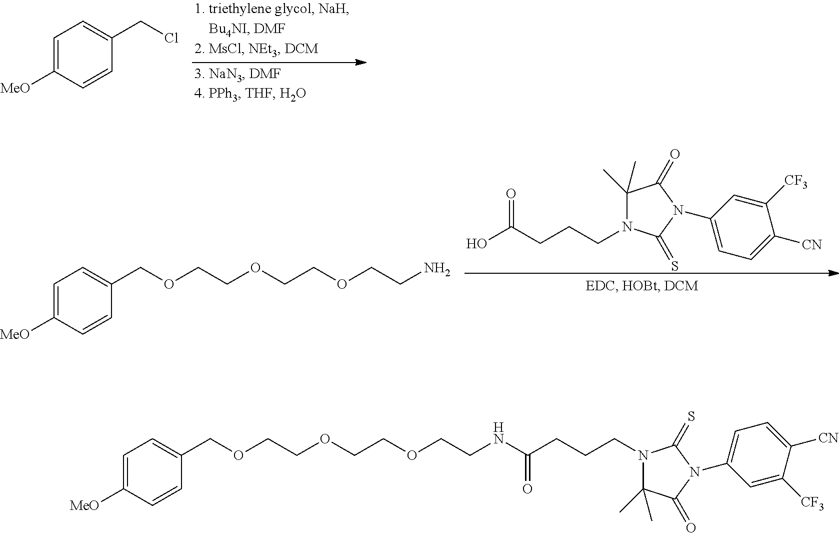 [SMILES: COc1ccc(CCl)cc1 ; CC1(C)C(=O)N(c2ccc(C#N)c(C(F)(F)F)c2)C(=S)N1CCCC(=O)O ; COc1ccc(COCCOCCOCCN)cc1 ; COc1ccc(COCCOCCOCCNC(=O)CCCN2C(=S)N(c3ccc(C#N)c(C(F)(F)F)c3)C(=O)C2(C)C)cc1]

### 2-(2-(2-((4-Methoxybenzyl)oxy)ethoxy)ethoxy)ethanol

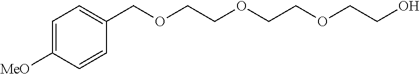 [SMILES: COc1ccc(COCCOCCOCCO)cc1]

Triethylene glycol (1.2 mL, 9 mmol, 3 eq) was dissolved in dry DMF (6 mL) in a 3 neck flask under argon and cooled to 4° C. 95% NaH (0.23 g, 9 mmol, 3 eq) was added, followed by 4-methoxybenzyl chloride (0.41 mL, 3 mmol, 1 eq). The solution was then warmed to room temperature and stirred for 14 hours. The reaction was then quenched with methanol, then diluted with 1M HCl and extracted twice with chloroform. The combined organic layer was dried over sodium sulfate, filtered and condensed. Purification by column chromatography (25 to 100% EtOAc/hexanes) gave a light yellow oil (0.27 g, 1.0 mmol, 33%). ¹H NMR (500 MHz, CDCl₃) δ 7.27 (d, J=8.4 Hz, 3H), 6.90-6.84 (m, 2H), 4.49 (s, 2H), 3.80 (s, 3H), 3.74-3.70 (m, 2H), 3.70-3.62 (m, 6H), 3.62-3.57 (m, 4H). ¹³C NMR (126 MHz, CDCl₃) δ 159.37, 130.35, 129.57, 113.92, 73.07, 72.66, 70.78, 70.77, 70.52, 69.18, 61.93, 55.41. MS (ESI) 287.0 (M+H₂O)⁺.

### 2-(2-(2-((4-Methoxybenzyl)oxy)ethoxy)ethoxy)ethyl methanesulfonate

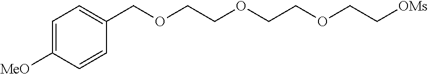 [SMILES: COc1ccc(COCCOCCOCCOS(C)(=O)=O)cc1]

2-(2-(2-((4-methoxybenzyl)oxy)ethoxy)ethoxy)ethanol (0.27 g, 1.0 mmol, 1 eq) was dissolved in DCM (2 mL) at room temperature. Triethylamine (0.42 mL, 3 mmol, 3 eq) was added, followed by methanesulfonyl chloride (0.12 mL, 1.5 mmol, 1.5 eq). After 19 hours, the mixture was diluted with 10% citric acid and extracted thrice with chloroform. The combined organic layer was dried over sodium sulfate, filtered and condensed. Purification by column chromatography (10 to 100% EtOAc/hexanes) gave a colorless oil (0.1598 g, 0.459 mmol, 46%). ¹H NMR (500 MHz, CDCl₃) δ 7.26-7.21 (m, 2H), 6.96-6.77 (m, 2H), 4.46 (s, 2H), 4.36-4.30 (m, 2H), 3.77 (s, 3H), 3.75-3.70 (m, 2H), 3.67-3.59 (m, 6H), 3.57 (dt, J=4.2, 1.5 Hz, 2H), 3.01 (s, 3H). ¹³C NMR (126 MHz, CDCl₃) δ 159.23, 130.24, 129.40, 113.78, 72.88, 70.63, 70.53, 69.36, 69.09, 69.00, 55.28, 37.65. MS (ESI) 371.4 (M+Na)⁺.

### 1-((2-(2-(2-Azidoethoxy)ethoxy)ethoxy)methyl)-4-methoxybenzene

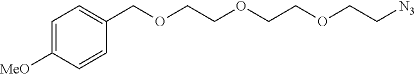 [SMILES: COc1ccc(COCCOCCOCCN=[N+]=[N-])cc1]

2-(2-(2-((4-methoxybenzyl)oxy)ethoxy)ethoxy)ethyl methanesulfonate (0.1598 g, 0.459 mmol, 1 eq) was dissolved in DMF (1.5 mL). Sodium azide (91 mg, 1.4 mmol, 3 eq) was added and the solution was heated to 100° C. After 14.5 hours, the mixture was diluted with water and extracted thrice with EtOAc. The combined organic layer was dried over sodium sulfate, filtered and condensed to give a yellow oil (0.1288 g, 0.4361 mmol, 95%) that was deemed sufficiently pure and carried forward to the next step. ¹H NMR (400 MHz, CDCl₃) δ 7.37-7.18 (m, 2H), 6.97-6.71 (m, 2H), 4.49 (s, 2H), 3.79 (s, 3H), 3.74-3.62 (m, 8H), 3.60 (dt, J=4.3, 1.3 Hz, 2H), 3.42-3.32 (m, 2H). MS (ESI) 318.1 (M+Na)⁺, 268.6 (M-N₂).

### 2-(2-(2-((4-Methoxybenzyl)oxy)ethoxy)ethoxy)ethanamine

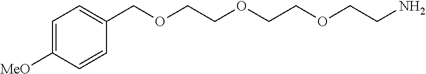 [SMILES: COc1ccc(COCCOCCOCCN)cc1]

1-((2-(2-(2-azidoethoxyl)ethoxy)ethoxy)methyl)-4-methoxybenzene (0.1288 g, 0.4361 mmol, 1 eq) was dissolved in THF (1.1 mL) at room temperature. Triphenylphosphine (0.172 g, 0.65 mmol, 1.5 eq) was added and the solution was stirred for 3.5 hours. Water (0.55 mL) was then added and the solution was stirred for 13.5 hours. The mixture was then diluted with diethyl ether, extracted twice with 1M HCl, basified with 3M NaOH and extracted 5 times with DCM. The combined organic layer was dried over sodium sulfate, filtered and condensed. Purification by column chromatography (1 to 20% 0.5N methanolic ammonia/DCM) gave a yellow oil (82.7 mg, 0.307 mmol, 70%). ¹H NMR (500 MHz, CDCl₃) δ 7.21 (t, J=5.7 Hz, 2H), 6.88-6.75 (m, 2H), 4.45 (s, 2H), 3.74 (s, 3H), 3.65-3.53 (m, 8H), 3.46 (t, J=5.2 Hz, 2H), 2.81 (s, 2H). ¹³C NMR (126 MHz, CDCl₃) δ 159.16, 130.28, 129.35, 113.72, 73.29, 72.85, 70.63, 70.56, 70.26, 69.07, 55.22, 41.70. MS (ESI) 270.2 (M+H)⁺.

### DB-7-216

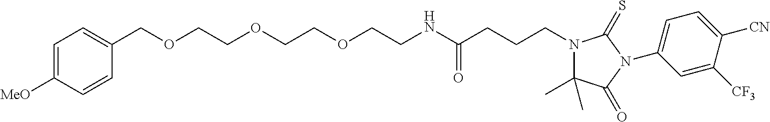 [SMILES: COc1ccc(COCCOCCOCCNC(=O)CCCN2C(=S)N(c3ccc(C#N)c(C(F)(F)F)c3)C(=O)C2(C)C)cc1]

4-(3-(4-cyano-3-(trifluoromethyl)phenyl)-5,5-dimethyl-4-oxo-2-thioxoimidazolidin-1-yl)butanoic acid (20 mg, 0.050 mmol, 1 eq), EDC (10.5 mg, 0.055 mmol, 1.1 eq) and HOBt (7.2 mg, 0.055 mmol, 1.1 eq) were dissolved in DCM (0.1 mL) at room temperature. DIPEA (9 μL, 0.050 mmol, 1 eq) was added and the solution was stirred for 15 minutes. 2-(2-(2-((4-methoxybenzyl)oxy)ethoxy)ethoxy)ethanamine (0.175 mL, 0.065 mmol, 1.3 eq) was added as a 100 mg/mL solution in DCM. After 17.5 hours, the mixture was diluted with 10 mL half saturated sodium chloride and extracted thrice with EtOAc. The combined organic layer was dried over sodium sulfate, filtered and condensed. Purification by preparative TLC (5% MeOH/DCM) gave a colorless oil (20.9 mg, 0.0321 mmol, 64%). ¹H NMR (500 MHz, CDCl₃) δ 7.94 (d, J=8.3 Hz, 1H), 7.89 (d, J=1.9 Hz, 1H), 7.76 (dd, J=8.2, 1.9 Hz, 1H), 7.27-7.24 (m, 3H), 6.93-6.82 (m, 2H), 6.40 (s, 1H), 4.49 (s, 2H), 3.80 (d, J=2.5 Hz, 3H), 3.77-3.71 (m, 2H), 3.69-3.60 (m, 8H), 3.59-3.55 (m, 2H), 3.45 (dd, J=10.2, 5.3 Hz, 2H), 2.27 (t, J=6.7 Hz, 2H), 2.17-2.06 (m, 2H), 1.60 (s, 6H). ¹³C NMR (126 MHz, CDCl₃) δ 178.48, 175.50, 171.87, 159.44, 137.27, 135.24, 133.59 (q, J=33.7 Hz), 132.21, 130.14, 129.58, 127.17 (q, J=4.7 Hz) 122.02 (q, J=274.2 Hz), 114.99, 113.96, 110.10, 73.09, 70.72, 70.67, 70.33, 69.88, 69.22, 65.45, 55.43, 43.74, 39.42, 32.85, 23.64, 23.20. MS (ESI) 651.4 (M+H)⁺.

### Example 28

### TMP Based Degraders

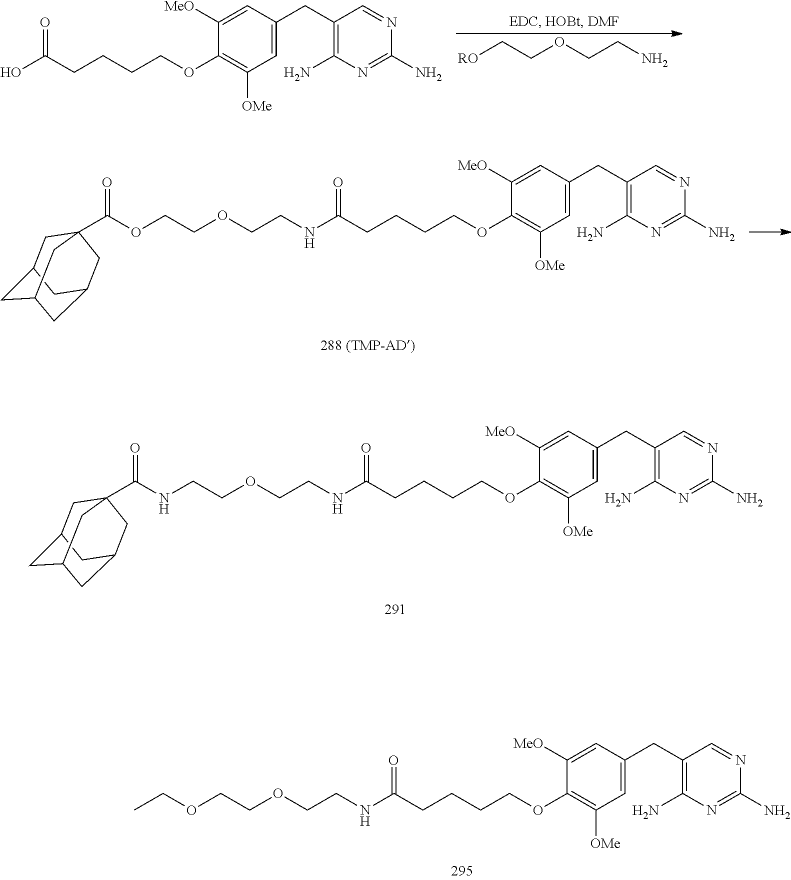

The TMP-acid (5-(4-((2,4-diaminopyrimidin-5-yl)methyl)-2,6-dimethoxyphenoxy)pentanoic acid) was synthesized as described by Cornish and coworkers (ChemBioChem, 2007, 8L767-774.)

### 2-(2-(5-(4-((2,4-Diaminopyrimidin-5-yl)methyl)-2,6-dimethoxyphenoxy) pentanamido)ethoxy)ethyl 2-(adamantan-1-yl)acetate (288-TMP-AD′)

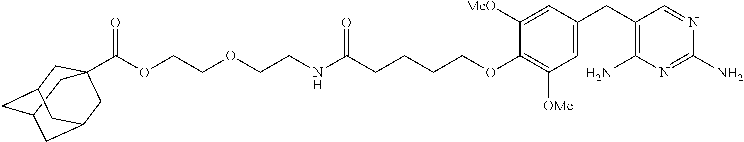 [SMILES: COc1cc(Cc2cnc(N)nc2N)cc(OC)c1OCCCCC(=O)NCCOCCOC(=O)C12CC3CC(CC(C3)C1)C2]

To a 1 dram vial with stirbar was charged 5-(4-((2,4-diaminopyrimidin-5-yl)methyl)-2,6-dimethoxyphenoxy)pentanoic acid (50.0 mg, 0.14 mmol), EDC (38.0 mg, 0.2 mmol), HOBt (31 mg, 0.2 mmol), and 1.3 mL DMF. After 15 minutes of stirring 2-(2-Aminoethoxyl)ethyl 2-(adamantan-1-yl)acetate (45 mg, 0.16 mmol) and Et₃N (46 μL, 4.0 mmol) were added and the mixture left stir for 16 h upon which the mixture was diluted with 2 mL EtOAc and washed with saturated NaHCO₃ (2×3 mL). The organic layer was dried with Na₂SO₄ and concentrated down to yield a crude oil which was purified by silica gel chromatography (DCM to 85:15 DCM:MeOH (0.5 N NH₃)) to yield 29 mg (45% yield) of pure product as an amber oil. ¹H NMR (500 MHz, CDCl₃) δ 7.72 (s, 1H), 6.36 (s, 2H), 5.01 (s, 2H), 4.83 (s, 2H), 4.23-4.11 (m, 2H), 3.94 (t, J=5.6, 2H), 3.78 (s, 6H), 3.64 (t, J=10.2, 6H), 3.54 (t, J=5.0, 2H), 3.50-3.40 (m, 2H), 2.29 (t, J=7.3, 2H), 1.95 (s, 3H), 1.89-1.73 (m, 4H), 1.69 (d, J=12.1, 3H), 1.61 (d, J=11.7, 9H); ¹³C NMR (126 MHz, CDCl₃) δ 173.14, 171.74, 162.84, 161.58, 155.25, 153.72, 135.90, 133.54, 106.4, 105.05, 72.87, 69.83, 69.05, 62.70, 56.12, 48.83, 42.33, 39.11, 36.68, 36.18, 34.60, 32.79, 29.38, 28.56, 22.45; LRMS (ESI) 640.3 (M+H)⁺.

### N-(2-(2-(5-(4-((2,4-Diaminopyrimidin-5-yl)methyl)-2,6-dimethoxyphenoxy) pentanamido)ethoxy)ethyl)adamantane-1-carboxamide (295)

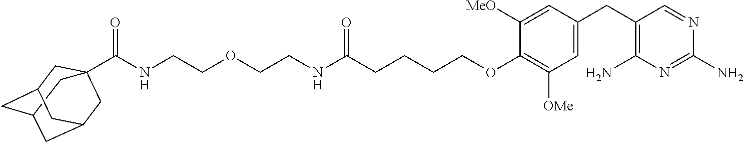 [SMILES: COc1cc(Cc2cnc(N)nc2N)cc(OC)c1OCCCCC(=O)NCCOCCNC(=O)C12CC3CC(CC(C3)C1)C2]

To a 1 dram vial with stirbar was charged 5-(4-((2,4-diaminopyrimidin-5-yl)methyl)-2,6-dimethoxyphenoxy)pentanoic acid (50.0 mg, 0.14 mmol), EDC (38.0 mg, 0.2 mmol), HOBt (31 mg, 0.2 mmol), and 1.3 mL DMF. After 15 minutes of stirring N-(2-(2-aminoethoxyl)ethyl)adamantane-1-carboxamide (48 mg, 0.17 mmol) and Et₃N (46 μL, 4.0 mmol) were added and the mixture was stirred for 16 h, upon which the mixture was diluted with 2 mL EtOAc and washed with saturated NaHCO₃ (2×3 mL). The organic layer was dried with Na₂SO₄ and concentrated down to yield a crude oil which was purified by silica gel chromatography (DCM to 85:15 DCM:MeOH (0.5 N NH₃)) to yield 29 mg (45% yield) of pure product as an amber oil. ¹H NMR (501 MHz, CDCl₃) δ 7.73 (s, 1H), 6.36 (s, 2H), 6.28 (s, 1H), 5.99 (s, 1H), 4.88 (s, 2H), 4.75 (s, 2H), 3.94 (t, J=6.1, 2H), 3.79 (d, J=18.4, 6H), 3.64 (d, J=12.3, 2H), 3.54-3.46 (m, 4H), 3.46-3.39 (m, 2H), 3.37 (dd, J=5.3, 10.4, 2H), 2.30 (t, J=7.2, 2H), 1.94 (s, 3H), 1.87-1.73 (m, 4H), 1.68 (d, J=12.1, 3H), 1.59 (m, 12H). ¹³C NMR (126 MHz, CDCl₃) δ 173.36, 171.27, 162.73, 162.03, 156.35, 153.66, 135.78, 133.82, 106.41, 105.01, 72.96, 69.84, 69.77, 56.10, 51.56, 42.55, 39.07, 38.98, 36.74, 36.11, 34.63, 32.71, 29.25, 28.61, 22.60; LRMS (ESI) 639.8 (M+H)⁺.

### 5-(4-((2,4-Diaminopyrimidin-5-yl)methyl)-2,6-dimethoxyphenoxy)-N-(2-(2-ethoxyethoxyl)ethyl)pentanamide (291)

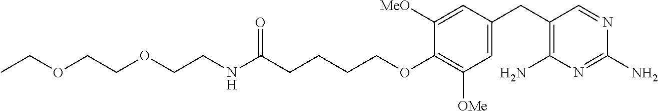 [SMILES: CCOCCOCCNC(=O)CCCCOc1c(OC)cc(Cc2cnc(N)nc2N)cc1OC]

To a 1 dram vial with stirbar was charged 5-(4-((2,4-diaminopyrimidin-5-yl)methyl)-2,6-dimethoxyphenoxy)pentanoic acid (38.0.0 mg, 0.1 mmol), EDC (28.0 mg, 0.15 mmol), HOBt (24 mg, 0.15 mmol), and 1.3 mL DMF. After 15 minutes of stirring 2-(2-ethoxyethoxyl)ethanamine (19 mg, 0.11 mmol) and Et₃N (46 μL, 4.0 mmol) were added and the mixture left stir for 16 h upon which the mixture was diluted with 2 mL EtOAc and washed with saturated NaHCO₃ (2×3 mL). The organic layer was dried with Na₂SO₄ and concentrated down to yield a crude oil which was purified by preparative TLC (DCM to 93:7 DCM:MeOH (0.5 N NH₃)) to yield 22 mg (45% yield) of pure product as an amber oil. ¹H NMR (501 MHz, CDCl₃) δ 7.70 (s, 1H), 6.30 (s, 2H), 6.13 (s, 1H), 4.70 (s, 2H), 4.53 (s, 2H), 3.88 (t, J=6.1, 2H), 3.73 (d, s, 6H), 3.61-3.33 (m, 12H), 2.21 (t, J=7.3, 2H), 1.77-1.64 (m, 4H), 1.15 (t, J=7.0, 3H); ¹³C NMR (126 MHz, CDCl₃) δ 173.06, 162.72, 162.04, 156.52, 153.77, 133.63, 132.60, 106.49, 105.00, 72.87, 70.26, 69.92, 69.69-56.14, 39.08, 36.28, 34.73, 29.45, 22.36, 15.15; LRMS (ESI) 491.6 (M+H)⁺.

### Example 29

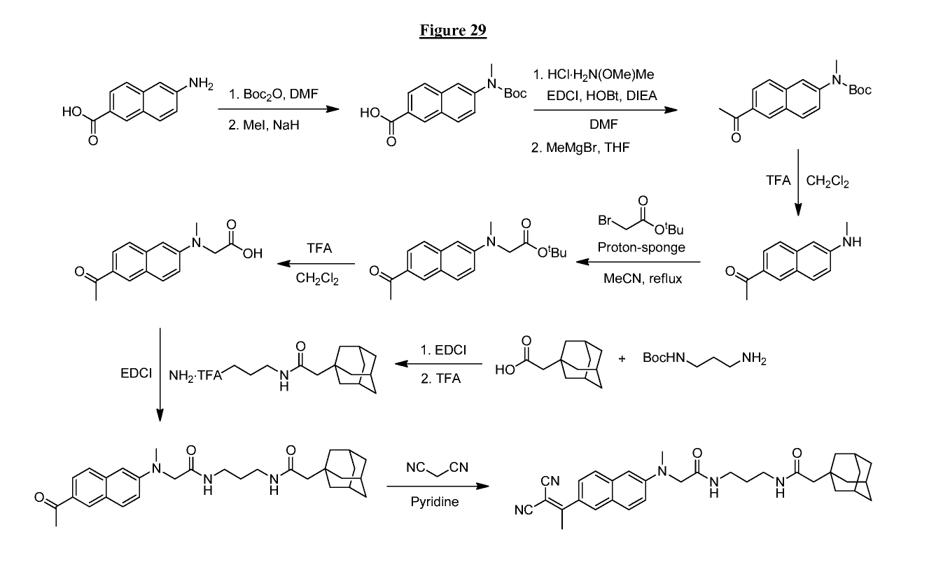

### 2-(Adamantan-1-yl)-N-(3-(2-((6-(1,1-dicyanoprop-1-en-2-yl)naphthalen-2-yl)(methyl)amino)acetamido)propyl)acetamide (FIG. 29)

### 6-((tert-Butoxycarbonyl)(methyl)amino)-2-naphthoic acid

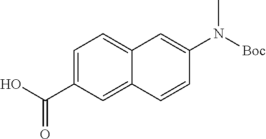 [SMILES: CN(C(=O)OC(C)(C)C)c1ccc2cc(C(=O)O)ccc2c1]

¹H NMR (500 MHz, CD₃OD) δ 8.55 (s, 1H), 8.02 (dd, J=8.6, 1.5 Hz, 1H), 7.95 (d, J=8.8 Hz, 1H), 7.87 (d, J=8.6 Hz, 1H), 7.76 (s, 1H), 7.50 (dd, J=8.8, 1.9 Hz, 1H), 3.39 (s, 3H), 3.35 (s, 3H), 1.45 (s, 9H).

### tert-Butyl (6-acetylnaphthalen-2-yl)(methyl)carbamate

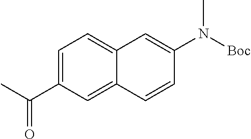 [SMILES: CC(=O)c1ccc2cc(N(C)C(=O)OC(C)(C)C)ccc2c1]

¹H NMR (400 MHz, CDCl₃) δ 8.42 (s, 1H), 8.00 (dd, J=8.6, 1.6 Hz, 1H), 7.90 (d, J=8.8 Hz, 1H), 7.82 (d, J=8.6 Hz, 1H), 7.66 (s, 1H), 7.53 (dd, J=8.8, 2.1 Hz, 1H), 3.39 (s, 3H), 2.71 (s, 3H), 1.48 (s, 9H). ¹³C NMR (100 MHz, CD₃OD) δ 197.9, 154.5, 143.8, 135.8, 134.1, 130.0, 129.7, 129.6, 128.0, 125.8, 124.3, 121.7, 80.9, 37.2, 28.3, 26.6. TLC (33% ethylacetate in hexanes), R_{f} 0.69 (UV, CAM).

### 1-(6-(Methylamino)naphthalen-2-yl)ethanone

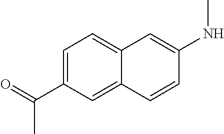

¹H NMR (400 MHz, CDCl₃) δ 8.30 (s, 1H), 7.93 (dd, J=8.6, 1.4 Hz, 1H), 7.71 (d, J=8.9 Hz, 1H), 7.63 (d, J=8.6 Hz, 1H), 6.91 (dd, J=8.8, 2.1 Hz, 1H), 6.78 (s, 1H), 3.17 (brs, 1H), 2.97 (s, 3H), 2.66 (s, 3H). ¹³C NMR (100 MHz, CD₃OD) δ 197.8, 149.0, 138.0, 130.8, 130.7, 130.4, 126.0, 125.9, 124.7, 118.4, 103.2, 30.5, 26.4. TLC (33% ethylacetate in hexanes), R_{f} 0.36 (UV, CAM).

### tert-Butyl 2-((6-acetylnaphthalen-2-yl)(methyl)amino)acetate

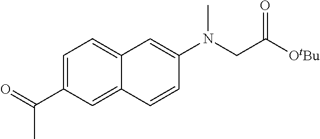 [SMILES: CC(=O)c1ccc2cc(N(C)CC(=O)OC(C)(C)C)ccc2c1]

To a stirred solution of 1-(6-(methylamino)naphthalen-2-yl)ethanone (31 mg, 0.10 mmol) in MeCN (1.5 mL) at rt were added tert-butyl bromoacetate (18 μL, 0.12 mmol) and proton-sponge (28 mg, 0.13 mmol). The reaction mixture was stirred at 82° C. for 16 h, cooled to rt, and concentrated. The residue was chromatographed (eluting with 5% ethylacetate in hexanes initially, grading to 20% ethylacetate in hexanes) on silica gel to give ester (29 mg, 94%). ¹H NMR (400 MHz, CDCl₃) δ 8.31 (s, 1H), 7.93 (d, J=8.7 Hz, 1H), 7.80 (d, J=9.0 Hz, 1H), 7.63 (d, J=8.7 Hz, 1H), 7.09 (d, J=9.0 Hz, 1H), 6.87 (d, J=2.6 Hz, 1H), 4.09 (s, 2H), 3.19 (s, 3H), 2.66 (s, 3H), 1.42 (s, 9H). ¹³C NMR (100 MHz, CD₃OD) δ 197.7, 169.6, 148.9, 137.5, 131.0, 130.8, 130.3, 126.3, 125.4, 124.5, 115.7, 105.7, 81.9, 55.2, 39.8, 28.0, 26.4. TLC (25% ethylacetate in hexanes), R_{f} 0.35 (UV, CAM).

### 2-((6-Acetylnaphthalen-2-yl)(methyl)amino)acetic acid

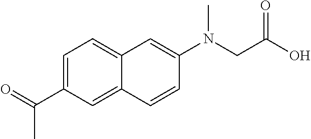 [SMILES: CC(=O)c1ccc2cc(N(C)CC(=O)O)ccc2c1]

To a stirred solution of amine (31 mg, 0.10 mmol) in MeCN (1.5 mL) at rt was added TFA (0.5 mL). The reaction mixture was stirred at 0° C. for 15 h, diluted with toluene (1.5 mL), and concentrated. The residue was chromatographed (eluting with 100% CH₂Cl₂ initially, grading to 7% CH₃OH in CH₂Cl₂) on silica gel to give 2-((6-acetylnaphthalen-2-yl)(methyl)amino)acetic acid (15.5 mg, quant.). ¹H NMR (400 MHz, CD₃OD) δ 8.38 (s, 1H), 7.85 (t, J=8.7 Hz, 2H), 7.63 (d, J=8.7 Hz, 1H), 7.17 (dd, J=9.2, 2.4 Hz, 1H), 6.92 (d, J=2.0 Hz, 1H), 4.26 (s, 2H), 3.17 (s, 3H), 2.63 (s, 3H). TLC (5% MeOH in CH₂Cl₂), R_{f} 0.09 (UV, CAM).

### 2-((6-Acetylnaphthalen-2-yl)(methylamino)-N-(3-(2-(adamantan-1-yl)acetamido)propyl)acetamide

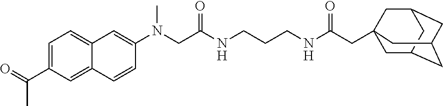 [SMILES: CC(=O)c1ccc2cc(N(C)CC(=O)NCCCNC(=O)CC34CC5CC(CC(C5)C3)C4)ccc2c1]

To a solution of 2((6-acetylnaphthalen-2-yl)(methyl)amino)acetic acid (10 mg, 0.039 mmol) in CH₂Cl₂ (1.5 mL) at rt were added amine (15 mg, 0.0408 mmol), HOBt (6.6 mg, 0.0469 mmol), and DIEA (35 uL, 0.1945 mmol). The mixture was cooled to 0° C. and EDCI (9 mg, 0.0469 mmol) was added to the mixture. The reaction mixture was stirred for 22 h at rt, cooled to 0° C., and quenched with water (2 mL). The resulting solution was extracted twice with ethylacetate. The combined extracts were washed with brine, dried over anhydrous Na₂SO₄, filtered, and concentrated. The residue was chromatographed (eluting with 5% ethylacetate in hexanes initially, grading to 25% ethylacetate in hexanes) on silica gel to give the pure 2((6-acetylnaphthalen-2-yl) (methyl)amino)-N-(3-(2-(adamantan-1-yl)acetamido)propyl)acetamide (15 mg, 79%). ¹H NMR (500 MHz, CD₃OD) δ 8.39 (s, 1H), 7.87 (d, J=2.0 Hz, 1H), 7.85 (s, 1H), 7.74 (brt, J=5.2 Hz, 1H), 7.65 (d, J=8.7 Hz, 1H), 4.09 (s, 2H), 3.24 (t, J=6.6 Hz, 2H), 3.21 (s, 3H), 3.10-3.06 (m, 2H), 2.63 (s, 3H), 1.88 (s, 3H), 1.85 (s, 2H), 1.68 (d, J=12.2 Hz, 3H), 1.65-1.57 (m, 5H), 1.54 (d, J=2.2 Hz, 6H). ¹³C NMR (126 MHz, CD₃OD) δ 200.3, 173.9, 172.9, 150.7, 139.1, 132.1, 132.0, 131.9, 127.5, 127.0, 125.2, 117.3, 107.0, 57.8, 51.9, 43.7, 40.1, 37.8, 37.5, 37.4, 33.7, 30.4, 30.1, 26.4. LRMS (ESI) 490.4 (M+H)⁺, TLC (33% ethylacetate in hexanes), R_{f} 0.22 (UV, CAM).

### 2-(Adamantan-1-yl)-N-(3-(2-((6-(1,1-dicyanoprop-1-en-2-yl)naphthalen-2-yl)(methyl)amino)acetamido)propyl)acetamide

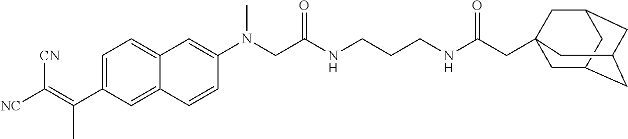 [SMILES: CC(=C(C#N)C#N)c1ccc2cc(N(C)CC(=O)NCCCNC(=O)CC34CC5CC(CC(C5)C3)C4)ccc2c1]

To a solution of 2-((6-acetylnaphthalen-2-yl)(methyl)amino)-N-(3-(2-(adamantan-1-yl)acetamido)propyl)acetamide (10 mg, 0.02 mmol) in pyridine (0.5 mL) at rt was added malononitrile (1 drop). The reaction mixture was stirred at 110° C. for 16 h, cooled to rt, and concentrated. The residue was chromatographed (eluting with 100% CH₂Cl₂ initially, grading to 7% CH₃OH in CH₂Cl₂) on silica gel to give the pure product (8.2 mg, 76%). ¹H NMR (500 MHz, CD₃OD) δ 8.16 (brt, J=5.8 Hz, 1H), 8.10 (d, J=1.7 Hz, 1H), 7.83 (d, J=9.1 Hz, 1H), 7.76 (brt, J=5.6 Hz, 1H), 7.73 (d, J=8.8 Hz, 1H), 7.60 (dd, J=8.8, 2.0 Hz, 1H), 7.19 (dd, J=9.1, 2.5 Hz, 1H), 6.98 (d, J=2.4 Hz, 1H), 4.10 (s, 2H), 3.25 (dt, J=5.7, 5.7 Hz, 2H), 3.23 (s, 3H), 3.10 (dt, J=6.3, 6.3 Hz, 2H), 2.69 (s, 3H), 1.87 (s, 3H), 1.86 (s, 2H), 1.69 (d, J=12.2 Hz, 3H), 1.65-1.57 (m, 5H), 1.55 (d, J=2.1 Hz, 6H). ¹³C NMR (126 MHz, CD₃OD) δ 176.9, 174.0, 173.9, 172.8, 150.8, 138.3, 131.6, 130.5, 130.1, 127.8, 127.1, 125.5, 117.7, 115.1, 114.7, 106.9, 82.3, 57.7, 54.8, 52.0, 51.9, 43.7, 40.1, 37.8, 37.5, 37.3, 33.7, 30.4, 30.1, 24.0. LRMS (ESI) 538.3 (M+H)⁺, TLC (5% MeOH in CH₂Cl₂), R_{f} 0.57 (UV, CAM).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a pharmaceutically acceptable salt, solvate or polymorph thereof:
L-DM (I), wherein:
DM is a protein degradation moiety;
L is a linker,
wherein L is covalently bound to DM;
L-DM has a ClogP value equal to or higher than about 1.5; and,
L comprises a functional group that is capable of forming a covalent bond to a protein binding moiety (PBM), wherein DM is selected from the group consisting of:

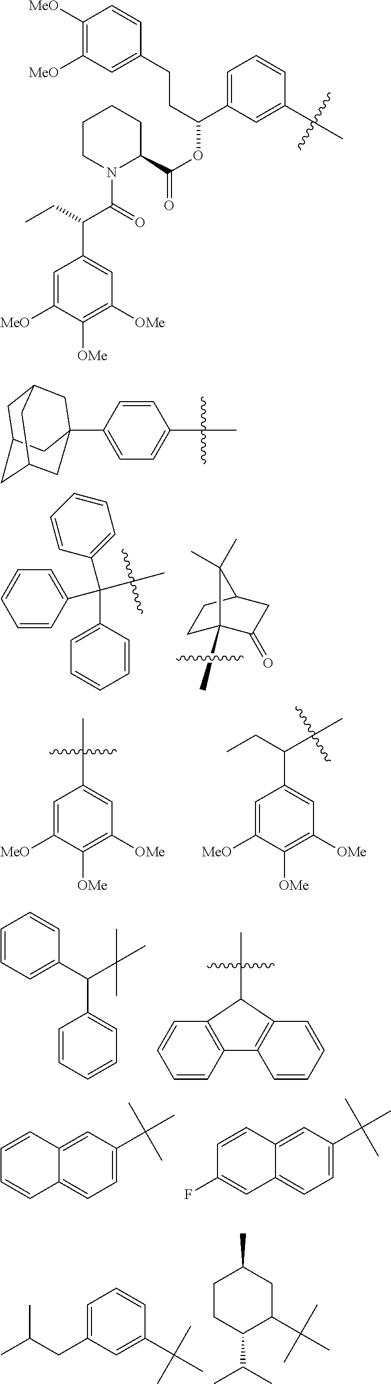

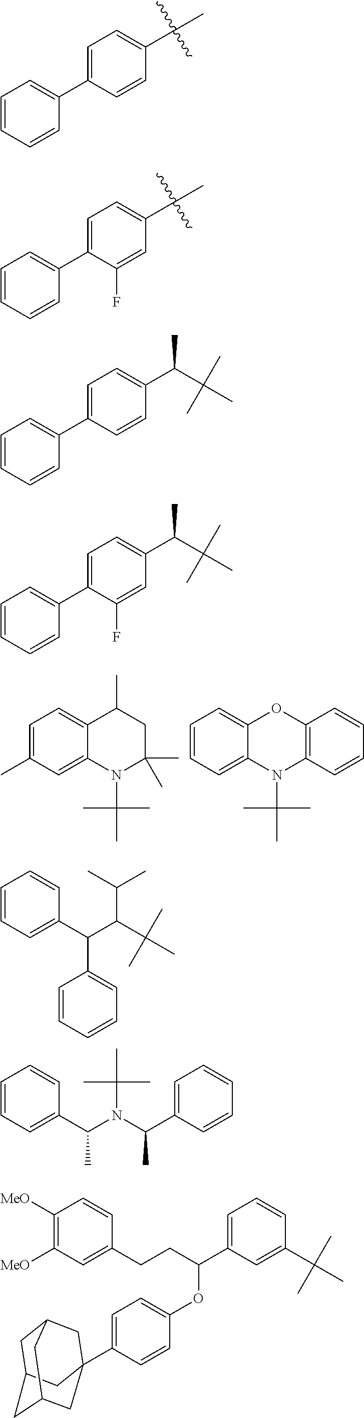

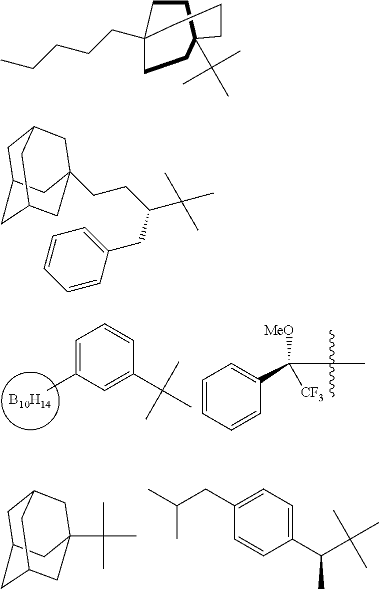

2-4. (canceled)

5. The compound of claim 1, wherein L ranges in length from 2 to 60 atoms.

6. (canceled)

7. The compound of claim 1, wherein L comprises from 1 to 15 ethylene oxide groups.

8. The compound of claim 1, wherein L comprises a group of formula (II):
—[Z—X—Y^{R}]— (II),
wherein Z links PBM to X; X links Z to group Y^{R}; and Y^{R} links to DM, further wherein:
Z and Y^{R} are independently a bond, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, —(CH₂)ᵢ—C≡C—, or —Y—C(O)—Y—;
X is -(D-CON-D)ᵢ-, wherein each occurrence of D is independently a bond, —(CH₂)ᵢ—Y—C(═O)—Y—(CH₂)ᵢ—, —(CH₂)ᵢ— or —[(CH₂)ᵢ—X¹]ᵢ—;
X¹ is O, S or N—R⁴;
CON is a bond, —C(O)NH—, —NH(CO)—, —X²—, —X³—C(O)—X³—, 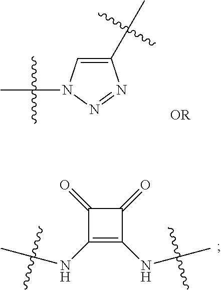 [SMILES: *c1cn(*)nn1 ; *Nc1c(N*)c(=O)c1=O]
X² is —O—, —S—, —N(R⁴)—, —S(O)—, —S(O)₂—, —S(O)₂O—, —OS(O)₂, or OS(O)₂O;
X³ is O, S, or NR⁴;
R⁴ is H or C₁-C₃ alkyl;
each occurrence of ‘i’ is independently an integer ranging from 0 to 100;
each occurrence of Y is independently a bond, O, S, —N(R^{N})—, —(CH₂)ᵢ—O, —(CH₂)ᵢ—S, —(CH₂)ᵢ—S(O)₂—, —(CH₂)ᵢ—N(R^{N})—, —(CH₂)ᵢ—XY—, or —(CH₂)ᵢ—C≡C—;
each occurrence of R^{N} is independently H, C₁-C₃ alkyl or hydroxylated C₁-C₃ alkyl; and,
XY is —C(O)NH—, —NHC(O), —OC(O)NH—, —NHC(O)O—, —C(O)O—, —OC(O)—, —C(O)S—, or —SC(O).

9. (canceled)

10. The compound of claim 8, wherein CON is —C(O)NH—, —NH(CO)—, or

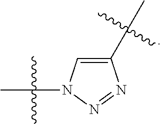 [SMILES: *c1cn(*)nn1]

11. The compound of claim 1, which is selected from the group consisting of: 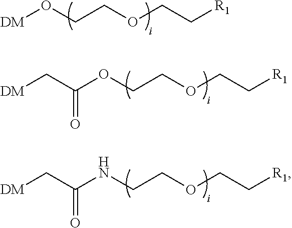
wherein R₁ is COOH, CHO, SH, OH or NH₂, and ‘i’ is an integer ranging from 1 to 100.

12. The compound of claim 11, which is selected from the group consisting of:

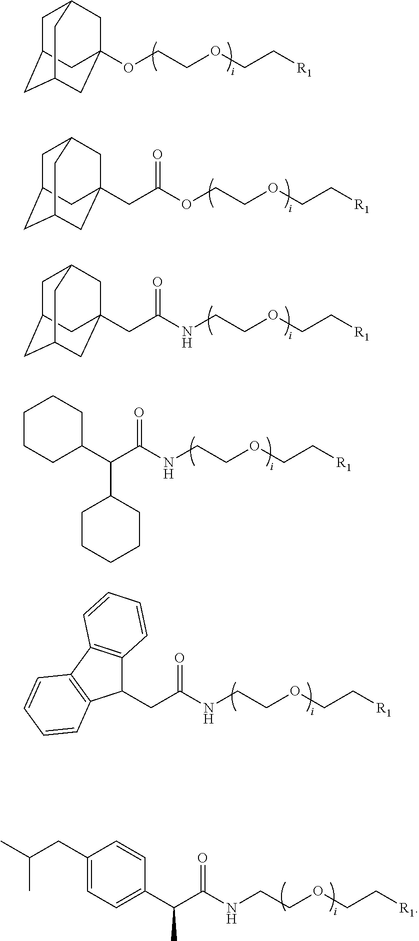

13. The compound ion of claim 1, wherein L is further covalently bound to a protein binding moiety (PBM), whereby the compound of formula (I) is the compound of formula (Ia) or a pharmaceutically acceptable salt, solvate or polymorph thereof:
PBM-L-DM (Ia).

14. The compound of claim 13, wherein the PBM binds to at least one target protein selected from the group consisting of an androgen receptor, sulfenic acid-comprising protein, a neurofibrillary tangle, and any combinations thereof.

15. The compound of claim 13, wherein the PBM is selected from the group consisting of: 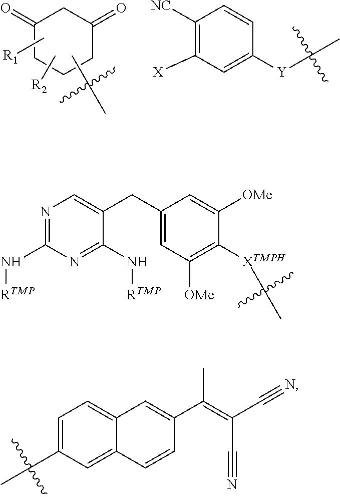
wherein:
each occurrence of R¹ and R² is independently selected from the group consisting of H, substituted C₁-C₆ alkyl, substituted C₂-C₆ alkynyl, —C(O)(C₁-C₆ alkyl), —NO₂, —CN, —F, —Cl, —Br, —I, —CF₃, —C(O)CF₃ and —C≡C—Rₐ, wherein each alkyl or alkynyl group is optionally and independently substituted with 1-6 electron withdrawing groups;
Rₐ is H or C₁-C₆ alkyl;
X is NO₂, CN, F, Cl, Br, I, —C≡C—Rₐ, CF₃, or —C(O)CF₃;
Y is a bond, 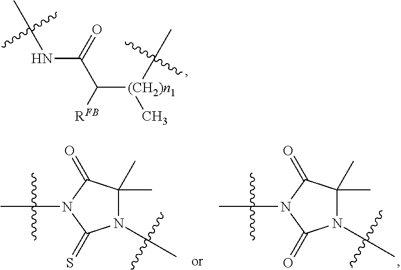
wherein
R^{FB} is H or OH, and n₁ is 0, 1, 2, or 3;
each occurrence of R^{TMP} is independently H, C₁-C₂₀ alkyl or C₁-C₂₀ acyl;
X^{TMP} is O, S, S(O)₂, CH₂ or NR^{FB1};
each occurrence of R^{FB1} is independently H or a C₁-C₃ alkyl group substituted with 1-3 hydroxyl groups; and,
each occurrence of n₂ is independently 0, 1, 2, or 3.

16-30. (canceled)

31. A method of treating or preventing a disease or disorder in a subject in need thereof, wherein the disease or disorder is associated with a target protein in the subject, the method comprising administering to the subject a therapeutically effective amount of a compound of formula (Ia) or a pharmaceutically acceptable salt, solvate or polymorph thereof:
PBM-L-DM (Ia),
wherein DM is a protein degradation moiety; L is a linker, wherein L is covalently bound to DM; L-DM has a total ClogP value equal to or higher than about 1.5; L is covalently bound to a protein binding moiety (PBM), and PBM binds to the target protein, wherein DM is selected from the group consisting of: 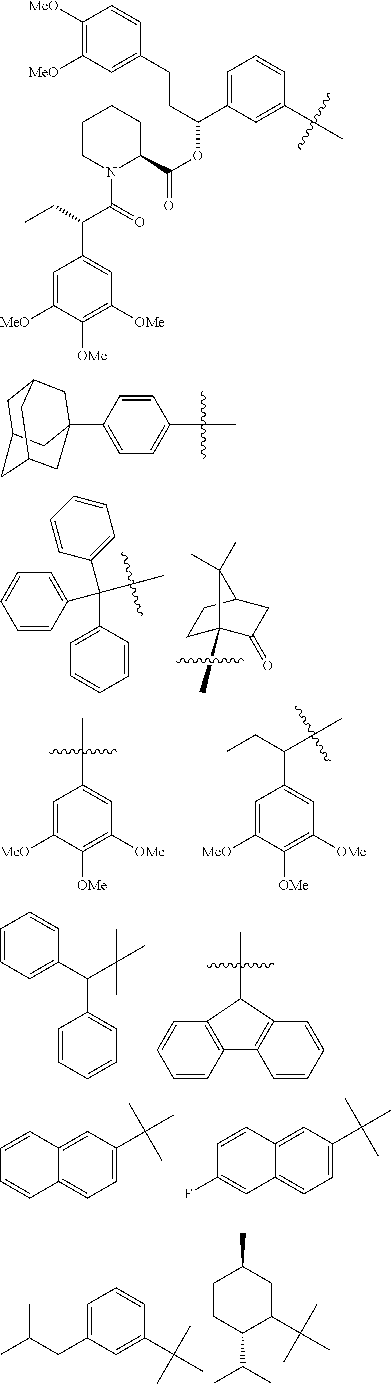 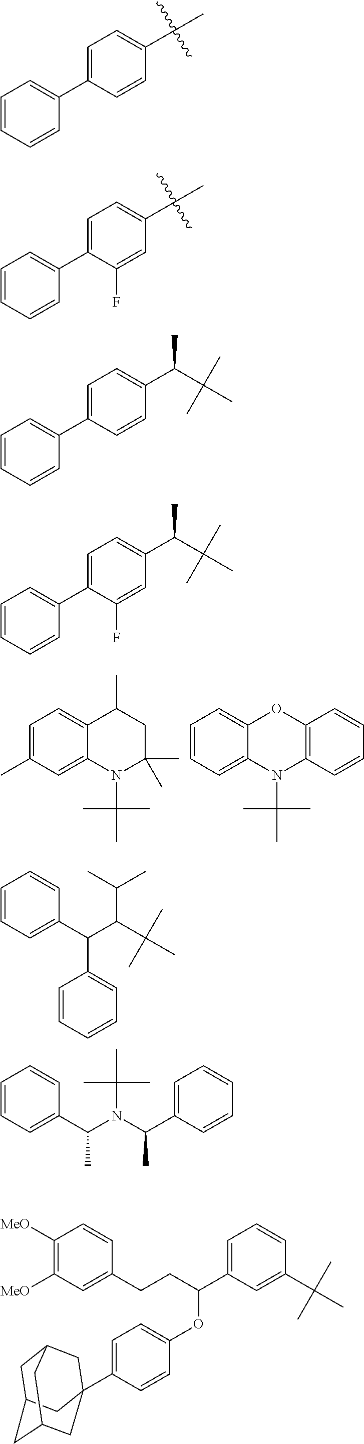 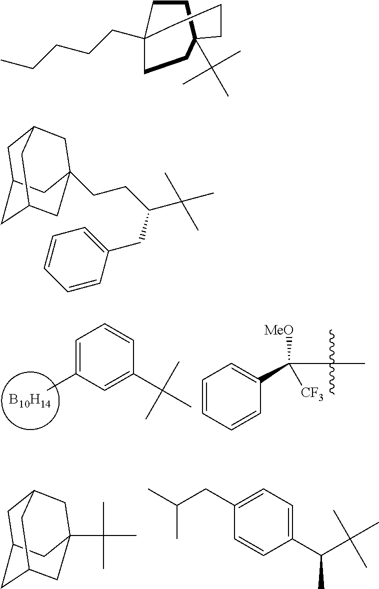
whereby the disease or disorder is treated or prevented in the subject.

32. The method of claim 31, wherein the disease or disorder comprises asthma, autoimmune diseases, cancers, ciliopathies, cleft palate, diabetes, heart disease, hypertension, inflammatory bowel disease, mental retardation, mood disorder, obesity, refractive error, infertility, Angelman syndrome, Canavan disease, coeliac disease, Charcot-Marie-Tooth disease, cystic fibrosis, duchenne muscular dystrophy, haemochromatosis, haemophilia, Klinefelter's syndrome, neurofibromatosis, phenylketonuria, polycystic kidney disease, (PKD1) or 4 (PKD2) Prader-Willi syndrome, sickle-cell disease, Tay-Sachs disease, Turner syndrome, Alzheimer's disease, amyotrophic lateral sclerosis (Lou Gehrig's disease), anorexia nervosa, anxiety disorder, atherosclerosis, attention deficit hyperactivity disorder, autism, bipolar disorder, chronic fatigue syndrome, chronic obstructive pulmonary disease, Crohn's disease, coronary heart disease, dementia, depression, diabetes mellitus type 1, diabetes mellitus type 2, epilepsy, Guillain-Barré syndrome, irritable bowel syndrome, lupus, metabolic syndrome, multiple sclerosis, myocardial infarction, obesity, obsessive-compulsive disorder, panic disorder, Parkinson's disease, psoriasis, rheumatoid arthritis, sarcoidosis, schizophrenia, stroke, thromboangiitis obliterans, Tourette syndrome, vasculitis, aceruloplasminemia, achondrogenesis type II, achondroplasia, acrocephaly, Gaucher disease type 2, acute intermittent porphyria, Canavan disease, adenomatous Polyposis Coli, ALA dehydratase deficiency, adenylosuccinate lyase deficiency, adrenogenital syndrome, adrenoleukodystrophy, ALA-D porphyria, ALA dehydratase deficiency, alkaptonuria, Alexander disease, alkaptonuric ochronosis, alpha 1-antitrypsin deficiency, alpha-1 proteinase inhibitor, emphysema, amyotrophic lateral sclerosis, Alström syndrome, Alexander disease, Amelogenesis imperfecta, ALA dehydratase deficiency, Anderson-Fabry disease, androgen insensitivity syndrome, anemia, angiokeratoma corporis diffusum, angiomatosis retinae (von Hippel-Lindau disease), Apert syndrome, arachnodactyly (Marfan syndrome), Stickler syndrome, arthrochalasis multiplex congenital (Ehlers-Danlos syndrome#arthrochalasia type), ataxia telangiectasia, Rett syndrome, primary pulmonary hypertension, Sandhoff disease, neurofibromatosis type II, Beare-Stevenson cutis gyrata syndrome, mediterranean fever, familial, Benjamin syndrome, beta-thalassemia, bilateral acoustic neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndrome (Rubinstein-Taybi syndrome), bronze diabetes/bronzed cirrhosis (hemochromatosis), bulbospinal muscular atrophy (Kennedy's disease), Burger-Grutz syndrome (lipoprotein lipase deficiency), CGD chronic granulomatous disorder, campomelic dysplasia, biotinidase deficiency, cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, chondrodystrophy syndrome (achondroplasia), otospondylomegaepiphyseal dysplasia, Lesch-Nyhan syndrome, galactosemia, Ehlers-Danlos syndrome, thanatophoric dysplasia, Coffin-Lowry syndrome, Cockayne syndrome (familial adenomatous polyposis), congenital erythropoietic porphyria, congenital heart disease, methemoglobinemia/congenital methaemoglobinaemia, achondroplasia, X-linked sideroblastic anemia, connective tissue disease, conotruncal anomaly face syndrome, Cooley's Anemia (beta-thalassemia), copper storage disease (Wilson's disease), copper transport disease (Menkes disease), hereditary coproporphyria, Cowden syndrome, craniofacial dysarthrosis (Crouzon syndrome), Creutzfeldt-Jakob disease (prion disease), Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, degenerative nerve diseases including de Grouchy syndrome and Dejerine-Sottas syndrome, developmental disabilities, distal spinal muscular atrophy, type V, androgen insensitivity syndrome, diffuse globoid body sclerosis (Krabbe disease), Di George's syndrome, dihydrotestosterone receptor deficiency, androgen insensitivity syndrome, Down syndrome, dwarfism, erythropoietic protoporphyria, erythroid 5-aminolevulinate synthetase deficiency, erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), fibrocystic disease of the pancreas, fragile X syndrome, galactosemia, genetic brain disorders, giant cell hepatitis (neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), hyperandrogenism, hypochondroplasia, hypochromic anemia, immune system disorders, Insley-Astley syndrome, Jackson-Weiss syndrome, Joubert syndrome, Lesch-Nyhan syndrome, Jackson-Weiss syndrome, kidney diseases, including hyperoxaluria, Klinefelter's syndrome, Kniest dysplasia, lacunar dementia, Langer-Saldino achondrogenesis, ataxia telangiectasia, Lynch syndrome, lysyl-hydroxylase deficiency, Machado-Joseph disease, metabolic disorders, Marfan syndrome, movement disorders, Mowat-Wilson syndrome, Muenke syndrome, multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford progeria syndrome), progressive chorea, chronic hereditary (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), recurrent polyserositis, retinal disorders, retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, skin pigmentation disorders, Smith-Lemli-Opitz syndrome, South-African genetic porphyria (variegate porphyria), infantile-onset ascending hereditary spastic paralysis, speech and communication disorders, sphingolipidosis, spinocerebellar ataxia, Stickler syndrome, stroke, androgen insensitivity syndrome, tetrahydrobiopterin deficiency, beta-thalassemia, thyroid disease, tomaculous neuropathy (hereditary neuropathy with liability to pressure palsies), Treacher Collins syndrome, triplo X syndrome (triple X syndrome), trisomy 21 (Down syndrome), trisomy X, VHL syndrome (von Hippel-Lindau disease), vision impairment and blindness (Alström syndrome), Vrolik disease, Waardenburg syndrome, Warburg Sjo Fledelius Syndrome, Weissenbacher-Zweymüller syndrome, Wolf-Hirschhorn syndrome, Wolff periodic disease, Weissenbacher-Zweymüller syndrome or Xeroderma pigmentosum.

33. The method of claim 32 wherein the disease or disorder comprises cancer.

34. The method of claim 33, wherein the cancer comprises squamous-cell carcinoma, basal cell carcinoma, adenocarcinoma, hepatocellular carcinomas, renal cell carcinomas, cancer of the bladder, bowel, breast, cervix, colon, esophagus, head, kidney, liver, lung, neck, ovary, pancreas, prostate, and stomach; leukemias; benign and malignant lymphomas; benign and malignant melanomas; myeloproliferative diseases; sarcomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor or teratocarcinomas.

35-50. (canceled)
